SEQUENCE LISTING (3 sequences)

SEQ ID NO: 1 - protein, 12 aa, Homo sapiens
  MOD_RES (3)..(3): site of chemical conjugation
WEYVPLGPFLGK

SEQ ID NO: 2 - protein, 14 aa, Homo sapiens
  MOD_RES (8)..(8): site of chemical modification
GDFVTSFYWPWQTK

SEQ ID NO: 3 - protein, 351 aa, Homo sapiens
MIVQRVVLNSRPGKNGNPVAENFRMEEVYLPDNINEGQVQVRTLYLSVDPYMRCRMNEDTGTDYITPWQLSQVVDGGGIGIIEESKHTNLTKGDFVTSFYWPWQTKVILDGNSLEKVDPQLVDGHLSYFLGAIGMPGLTSLIGIQEKGHITAGSNKTMVVSGAAGACGSVAGQIGHFLGCSRVVGICGTHEKCILLTSELGFDAAINYKKDNVAEQLRESCPAGVDVYFDNVGGNISDTVISQMNENSHIILCGQISQYNKDVPYPPPLSPAIEAIQKERNITRERFLVLNYKDKFEPGILQLSQWFKEGKLKIKETVINGLENMGAAFQSMMTGGNIGKQIVCISEEISL

(19) United States
(12) Patent Application Publication
(10) Pub. No.: US 2024/0210412 A1
(43) Pub. Date: Jun. 27, 2024

(54) SULFONYL-TRIAZOLE COMPOUNDS USEFUL AS LIGANDS AND INHIBITORS OF PROSTAGLANDIN REDUCTASE 2

(71) Applicant: University of Virginia Patent Foundation, Charlottesville, VA (US)

(72) Inventors: Ku-Lung Hsu, Charlottesville, VA (US); Anthony Michael Ciancone, Bethesda, MD (US); Robert Justin Grams, Waynesboro, VA (US)

(73) Assignee: University of Virginia Patent Foundation, Charlottesville, VA (US)

(21) Appl. No.: 18/286,978

(22) PCT Filed: Apr. 13, 2022
(86) PCT No.: PCT/US22/24685

§ 371 Date: Oct. 13, 2023

Related U.S. Application Data
(60) Provisional application No. 63/174,466, filed on Apr. 13, 2021.

(51) Int. Cl. G01N 33/68; C07D 249/08; C07D 401/04; C07D 401/12; C07D 405/04; G01N 33/58

(52) U.S. Cl. CPC G01N 33/6848; C07D 249/08; C07D 401/04; C07D 401/12; C07D 405/04; G01N 33/58

(57) ABSTRACT

Sulfonyl-triazole compounds and related sulfonyl-heterocycle compounds are described. Exemplary compounds can form covalent adducts with reactive nucleophilic amino acid residues in proteins, such as reactive tyrosines, to form modified proteins and/or to alter the biological activity of the proteins. Pharmaceutical compositions comprising the compounds and methods of inhibiting prostaglandin reductase 2 (PTGR2) are also described. In addition, methods are described for screening proteins to identify druggable amino acid residues, e.g., druggable tyrosine and/or lysine residues.

### CROSS-REFERENCE TO RELATED APPLICATION

This application claims the benefit of U.S. Provisional Patent Application Ser. No. 63/174,466, filed Apr. 13, 2021, the disclosure of which is incorporated herein by reference in its entirety.

### GRANT STATEMENT

This invention was made with government support under Grant No. DA043571, awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062-155_PCT_ST25.txt; Size: 4 kilobytes; and Date of Creation: Apr. 13, 2022) filed with the instant application is incorporated herein by reference in its entirety.

### TECHNICAL FIELD

The presently disclosed subject matter relates to sulfonyl-heterocycle compounds, such as sulfonyl-triazole compounds, and to related pharmaceutical compositions. The presently disclosed subject matter also relates to methods of identifying a reactive tyrosine or a reactive lysine in a protein and to methods of inhibiting prostaglandin reductase 2 (PTGR2).

### INTRODUCTION

Recent advancements in chemoproteomics has furnished new chemical tools for investigating protein function on a global scale^{[1]}. Electrophilic compounds serve as the basis for developing activity-based probes (ABPs) and ligands to facilitate the investigation of biochemical and signaling functions of proteins in complex biological systems^{[2]}. These efforts have produced covalent probes^{[3]} and drug candidates^{[4]} for focused studies on specific classes of proteins (e.g. serine hydrolases^{[5]}) as well as reactive chemistry to functionally profile nucleophilic residues including lysine^{[6]}, cysteine^{[7]}, and tyrosines^{[8]}. Chemoproteomics can also inform on inhibitor activity through competitive assays to evaluate potency and selectivity of lead inhibitors and clinical candidates in the complex proteome. Despite these advances, however, a large fraction of the human proteome lacks pharmacological probes^{[9]}.

Accordingly, there is an ongoing need for additional compounds for covalent modification of nucleophilic amino acid sites, e.g., for use in developing compounds to modulate protein function.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a compound having a structure of Formula (I):

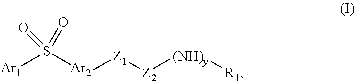

wherein: y is 0 or 1; Ar₁ is selected from the group comprising triazole, substituted triazole, imidazole, substituted imidazole, pyrazole, substituted pyrazole, tetrazole, and substituted tetrazole; Ar₂ is aryl or heteroaryl; Z₁ is —CH₂— or —C(═O)—; Z₂ is a heterocyclic, heteroaryl, substituted heterocyclic or substituted heteroaryl group; and R₁ is aryl, substituted aryl, heteroaryl or substituted heteroaryl; subject to the proviso that when Z₂ is piperidinyl, y is 1 and that when Z₁ is —C(═O)— and Z₂ is piperazinyl, R₁ is not substituted pyrimidinyl; or a pharmaceutically acceptable salt thereof.

In some embodiments, Ar₂ is selected from phenyl, pyridinyl, pyrimidinyl, and triazinyl. In some embodiments, Ar₁ is 1,2,4-triazole or a substituted 1,2,4-triazole group. In some embodiments, Z₂ is selected from the group comprising piperazinyl, piperidinyl, tetrahydrofuranyl, pyrrolidinyl, pyrrolyl, furanyl, diazepanyl, azetidinyl, and 2,6-diaza[3.3]heptanyl. In some embodiments, R₁ is an optionally substituted aryl or heteroaryl group, wherein said aryl or heteroaryl group is selected from phenyl, pyridyl, indolinyl, tetrahydroquinolinyl, indolyl, benzofuranyl, indanyl, dihydrobenzofuranyl, chromanyl, benzofuranyl, anthranilyl, benzofurazanyl, isoindolyl, oxindolyl, and isocarbostyrilyl.

In some embodiments, the compound having a structure of Formula (I) has a structure of Formula (II):

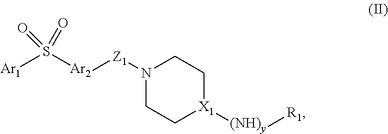

wherein: y is 0 or 1; Ar₁ is selected from the group comprising triazole, substituted triazole, imidazole, substituted imidazole, pyrazole, substituted pyrazole, tetrazole, and substituted tetrazole; Ar₂ is aryl or heteroaryl; Z₁ is —CH₂— or —C(═O)—; X₁ is N or CH; and R₁ is aryl, substituted aryl, heteroaryl or substituted heteroaryl; subject to the proviso that when X₁ is CH, y is 1 and when X₁ is N, y is 0; or a pharmaceutically acceptable salt thereof. In some embodiments, Z₁ is —C(═O)—.

In some embodiments, the compound having a structure of Formula (I) has a structure of Formula (III):

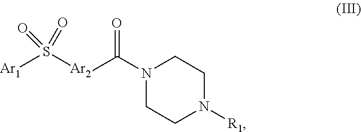

wherein: Ar₁ is selected from the group comprising triazole, substituted triazole, imidazole, substituted imidazole, pyrazole, substituted pyrazole, tetrazole, and substituted tetrazole; Ar₂ is aryl or heteroaryl; and R₁ is aryl, heteroaryl, substituted aryl, or substituted heteroaryl, optionally phenyl, substituted phenyl, pyridyl, or substituted pyridyl; or a pharmaceutically acceptable salt thereof. In some embodiments, Ar₂ is phenyl and the compound of Formula (III) has a structure of Formula (IIIa) or Formula (IIIb):

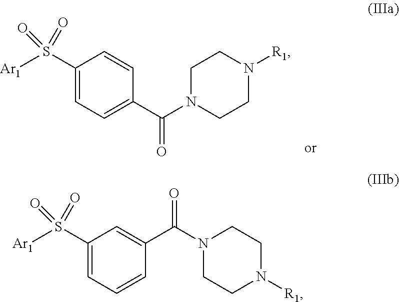

wherein: Ar₁ is selected from the group comprising triazole, substituted triazole, imidazole, substituted imidazole, pyrazole, substituted pyrazole, tetrazole, and substituted tetrazole; and R₁ is aryl, heteroaryl, substituted aryl, or substituted heteroaryl, optionally phenyl, substituted phenyl, pyridyl, or substituted pyridyl; or a pharmaceutically acceptable salt thereof.

In some embodiments, Ar₁ is a 1,2,4-triazole or substituted 1,2,4-triazole group and the compound of Formula (III) has a structure of Formula (IV):

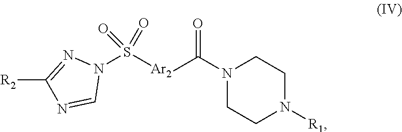

wherein: Ar₂ is aryl or heteroaryl; R₁ is aryl, substituted aryl, heteroaryl, or substituted heteroaryl, optionally phenyl, substituted phenyl, pyridyl, or substituted pyridyl; and R₂ is selected from H, alkyl, cycloalkyl, aryl, and substituted aryl; or a pharmaceutically acceptable salt thereof. In some embodiments, the compound of Formula (IV) has a structure of Formula (IVa) or (IVb):

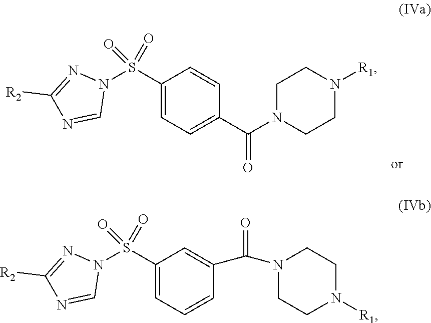

wherein: R₁ is phenyl, substituted phenyl, pyridyl, or substituted pyridyl; and R₂ is selected from H, alkyl, cycloalkyl, aryl, and substituted aryl; or a pharmaceutically acceptable salt thereof.

In some embodiments, R₂ is selected from the group comprising H, cycloalkyl, phenyl, furanyl, pyridyl, and substituted phenyl. In some embodiments, R₂ is substituted phenyl, wherein said substituted phenyl is phenyl substituted with one or more substituent selected from the group comprising halo, perfluoroalkyl, alkoxy, perfluoralkoxy, and aryl. In some embodiments, R₂ is selected from H, 2-pyridyl, and 2-methoxyphenyl.

In some embodiments, R₁ is substituted phenyl or substituted pyridyl, wherein said substituted phenyl or substituted pyridyl are phenyl or pyridyl substituted with one or more substituent selected from the group comprising alkyl, halo, haloalkyl, alkoxy, acyl, —C(═O)—NH₂, amino, alkylamino, and dialkylamino, optionally alkyl, halo, and alkoxy. In some embodiments, R₁ is substituted phenyl, optionally wherein R₁ is phenyl substituted by one or more substituent selected from halo and alkoxy. In some embodiments, R₁ is alkoxy-substituted phenyl, optionally methoxy-substituted phenyl. In some embodiments, R₁ is 2-methoxyphenyl.

In some embodiments, the compound is selected from the group comprising: (4 ((1H-1,2,4-triazol-1-yl)sulfonyl)phenyl)(4-phenylpiperazin-1-yl)methanone (AMC-0702); (4-((1H-1,2,4-triazol-1-yl)sulfonyl)phenyl)(4-(2-methoxyphenyl)piperazin-1-yl)methanone (AMC-0703); (4-(2-methoxyphenyl)piperazin-1-yl)(4-((3-phenyl-1H-1,2,4-triazol-1-yl)sulfonyl)-phenyl)methanone (RJG-1101); (4-((1H-1,2,4-triazol-1-yl)-sulfonyl)phenyl)(4-(3-methoxyphenyl)piperazin-1-yl)-methanone (RJG-1103); (4-((1H-1,2,4-triazol-1-yl)sulfonyl)phenyl)(4-(4-methoxyphenyl)piperazin-1-yl)-methanone (RJG-1105); (4-((3-(4-bromophenyl)-1H-1,2,4-triazol-1-yl)sulfonyl)phenyl)(4-(2-methoxy-phenyl)piperazin-1-yl)methanone (RJG-1112); (4-((3-(4-fluorophenyl)-1H-1,2,4-triazol-1-yl)sulfonyl)phenyl)(4-(2-methoxy-phenyl)piperazin-1-yl)methanone (RJG-1114); (4-(2-methoxyphenyl)piperazin-1-yl)(4-((3-(4-(trifluoromethyl)phenyl)-1H-1,2,4-triazol-1-yl)-sulfonyl)phenyl)methanone (RJG-1115); (4-((3-(furan-2-yl)-1H-1,2,4-triazol-1-yl)sulfonyl)phenyl)(4-(2-methoxyphenyl)-piperazin-1-yl)methanone RJG-1185, (4-((3-(2-fluorophenyl)-1H-1,2,4-triazol-1-yl)sulfonyl)phenyl)(4-(2-methoxy-phenyl)piperazin-1-yl)methanone (RJG-1187); (4-((3-(3-fluorophenyl)-1H-1,2,4-triazol-1-yl)sulfonyl)phenyl)(4-(2-methoxy-phenyl)piperazin-1-yl)methanone (RJG-1188); (4-(2-methoxyphenyl)piperazin-1-yl)(4-((3-(pyridin-3-yl)-1H-1,2,4-triazol-1-yl)-sulfonyl)-phenyl)methanone (RJG-1189); (4-(2-methoxyphenyl)piperazin-1-yl)(4-((3-(4-(trifluoromethoxy)phenyl)-1H-1,2,4-triazol-1-yl)sulfonyl)phenyl)methanone (RJG-1228); (4-(4-iodophenyl)piperazin-1-yl)(4-((3-phenyl-1H-1,2,4-triazol-1-yl)sulfonyl)-phenyl)-methanone (RJG-1257); (4-((1H-1,2,4-triazol-1-yl)sulfonyl)phenyl)(4-(pyridin-2-yl)-piperazin-1-yl)-methanone (RJG-1285); (4-(2-methoxyphenyl)piperazin-1-yl)(4-((3-(pyridin-4-yl)-1H-1,2,4-triazol-1-yl)-sulfonyl)phenyl)methanone (RJG-1291); (4-((3-([1,1′-biphenyl]-4-yl)-1H-1,2,4-triazol-1-yl)sulfonyl)phenyl)(4-(2-methoxy-phenyl)-piperazin-1-yl)methanone (RJG-2011); (3-((1H-1,2,4-triazol-1-yl)sulfonyl)-phenyl)(4-(2-methoxyphenyl)piperazin-1-yl)-methanone (RJG-2036); (4-((3-cyclopropyl-1H-1,2,4-triazol-1-yl)sulfonyl)phenyl)(4-(2-methoxyphenyl)-piperazin-1-yl)methanone (RJG-2048); 4-((3-cyclobutyl-1H-1,2,4-triazol-1-yl)sulfonyl)phenyl)(4-(2-methoxyphenyl)-piperazin-1-yl)methanone (RJG-2049); (4-((3-cyclopentyl-1H-1,2,4-triazol-1-yl)sulfonyl)phenyl)(4-(2-methoxyphenyl)-piperazin-1-yl)methanone (RJG-2050); (4-((3-cyclohexyl-1H-1,2,4-triazol-1-yl)sulfonyl)phenyl)(4-(2-methoxyphenyl)-piperazin-1-yl)methanone (RJG-2051); (4-((3-(4-methoxyphenyl)-1H-1,2,4-triazol-1-yl)sulfonyl)phenyl)(4-(2-methoxy-phenyl)piperazin-1-yl)methanone (RJG-2056); and (4 ((1H-1,2,4-triazol-1-yl)sulfonyl)phenyl)(4-((2-methoxyphenyl)amino)piperidin-1-yl)-methanone (RJG-2058); or a pharmaceutically acceptable salt thereof.

In some embodiments, the compound is selected from (4-((1H-1,2,4-triazol-1-yl)sulfonyl)phenyl)(4-(2-methoxyphenyl)piperazin-1-yl)methanone (AMC-0703), (4-(2-methoxyphenyl)piperazin-1-yl)(4-((3-(pyridin-3-yl)-1H-1,2,4-triazol-1-yl)-sulfonyl)-phenyl)methanone (RJG-1189), and (3-((1H-1,2,4-triazol-1-yl)sulfonyl)phenyl)-(4-(2-methoxyphenyl)piperazin-1-yl)-methanone (RJG-2036); or a pharmaceutically acceptable salt thereof.

In some embodiments, the presently disclosed subject matter provides a pharmaceutical composition comprising a compound of Formula (I) and a pharmaceutically acceptable carrier.

In some embodiments, the presently disclosed subject matter provides a method of inhibiting prostaglandin reductase 2 (PTGR2), wherein the method comprises contacting a sample comprising PTGR2 with an effective amount of a compound of Formula (I) or a pharmaceutical composition thereof. In some embodiments, the sample comprising PTGR2 is a biological sample selected from a biological fluid, a cell culture, a cell extract, a tissue, a tissue extract, an organ, or an organism.

In some embodiments, the presently disclosed subject matter provides a probe compound, wherein the probe compound has a structure of Formula (V):

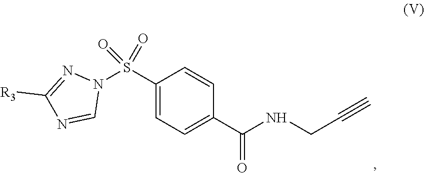

wherein: R₃ is selected from cyclopropyl, cyclobutyl, cyclopentyl, and cyclohexyl.

In some embodiments, the presently disclosed subject matter provides a method of identifying a reactive tyrosine and/or a reactive lysine of a protein, the method comprising: (a) providing a protein sample comprising isolated proteins, living cells, or a cell lysate; (b) contacting the protein sample with a probe compound of Formula (V) for a period of time sufficient for the probe compound to react with at least one reactive tyrosine and/or at least one reactive lysine in a protein in the protein sample, thereby forming at least one modified reactive tyrosine and/or at least one modified reactive lysine residue; and (c) analyzing proteins in the protein sample to identify at least one modified tyrosine residue and/or at least one modified lysine residue, thereby identifying at least one reactive tyrosine and/or at least one reactive lysine of a protein; wherein the at least one modified reactive tyrosine residue comprises a modified tyrosine residue comprising a structure of Formula (VI):

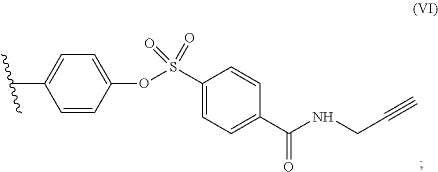 [SMILES: *c1ccc(OS(=O)(=O)c2ccc(C(=O)NCC#C)cc2)cc1]

and the at least one modified reactive lysine residue comprises a modified lysine residue comprising a structure of Formula (VII):

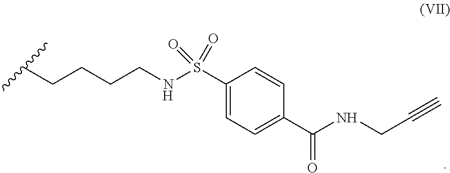

In some embodiments, the analyzing of step (c) further comprises tagging the at least one modified reactive tyrosine residue and/or the at least one modified reactive lysine residue with a compound comprising a detectable labeling group, thereby forming at least one tagged reactive tyrosine residue comprising said detectable labeling group and/or at least one tagged reactive lysine residue comprising said detectable labeling group, optionally wherein the detectable labeling group comprises biotin or a biotin derivative, optionally wherein the biotin derivative is desthiobiotin. In some embodiments, the tagging comprises reacting an alkyne group of the at least one tagged reactive tyrosine residue and/or at least one tagged lysine residue with a compound comprising an (i) an azide moiety and (ii) the detectable labeling group, optionally via a copper-catalyzed azide-alkyne cycloaddition (CuAAC) coupling reaction. In some embodiments, the analyzing further comprises digesting the protein sample with trypsin to provide a digested protein sample comprising a protein fragment comprising the at least one tagged reactive tyrosine moiety comprising the detectable group and/or a protein fragment comprising the at least one tagged reactive lysine moiety comprising the detectable group. In some embodiments, the analyzing further comprises enriching the digested protein sample for the detectable labeling group, optionally wherein the enriching comprises contacting the digested protein sample with a solid support comprising a binding partner of the detectable labeling group. In some embodiments, the analyzing further comprises analyzing the enriched the digested protein sample via liquid chromatography-mass spectrometry.

In some embodiments, the presently disclosed subject matter provides a compound having a structure of Formula (I′):

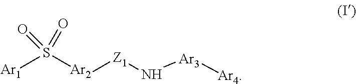

wherein: Ar₁ is selected from the group comprising triazole, substituted triazole, imidazole, substituted imidazole, pyrazole, substituted pyrazole, tetrazole, and substituted tetrazole; Ar₂ is aryl or heteroaryl; Z₁ is —CH₂— or —C(═O)—; Ar₃ is aryl or heteroaryl; and Ar₄ is substituted aryl, heteroaryl, or substituted heteroaryl; or a pharmaceutically acceptable salt thereof. In some embodiments, Ar₁ is 1,2,4-triazole or substituted 1,2,4-triazole. In some embodiments, Z₁ is —C(═O)—.

In some embodiments, Ar₃ is phenyl. In some embodiments, Ar₄ is substituted phenyl or pyridyl. In some embodiments, the compound is selected from the group comprising: 4-((1H-1,2,4-triazol-1-yl)sulfonyl)-N-(2′-methoxy-[1,1′-biphenyl]-4-yl)benzamide (RJG-2040A), 4-((1H-1,2,4-triazol-1-yl)sulfonyl)-N-(2′-(trifluoromethoxy)-[1,1′-biphenyl]-4-yl)-benzamide (RJG-2040B), and 4-((1H-1,2,4-triazol-1-yl)sulfonyl)-N-(4-(pyridin-2-yl)phenyl)benzamide (RJG-2040C); or a pharmaceutically acceptable salt thereof.

In some embodiments, the presently disclosed subject matter provides a pharmaceutical composition comprising a compound of Formula (I′) and a pharmaceutically acceptable carrier.

Accordingly, it is an object of the presently disclosed subject matter to provide compounds of Formula (I) (II), (III), (IIIa), (IIIb), (IV), (IVa), (IVb), and (I′), pharmaceutical compositions thereof, related probes, and related methods of inhibiting PTGR2 and of detecting a reactive tyrosine and/or a reactive lysine in a protein. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTIONS OF THE FIGURES

FIG. 1 is a schematic diagram showing a general scheme for the synthesis of exemplary sulfonyl-triazole exchange (SuTEx) ligands.

FIG. 2 is a schematic diagram showing an experimental workflow for quantitative liquid chromatography-mass spectrometry (LC-MS) chemoproteomic evaluation of sulfonyl-triazole exchange (SuTEx) ligand activity and selectivity. The stable isotope labeling by amino acids in cell culture (SILAC) ratio (SR) is a quantification of the area under the curve (AUC) of first mass spectrometer (MS1) extracted ion chromatograms (EIC) from SuTEx probe HHS-475 labeled peptides in dimethyl sulfoxide (DMSO) control (“light”) compared with ligand-treated (“heavy”) proteomes.

FIGS. 3A-3C: Activity-based protein profiling (ABPP) of sulfonyl-triazole exchange (SuTEx) ligands in human sarcomatoid mesothelioma (DM93) cells. FIG. 3A is a heatmap representation of stable isotope labeling by amino acids in cell culture (SILAC) ratios (SR) of tyrosine sites competed by fragments and organized by hierarchical clustering. Competition with SuTEx ligands was performed as shown in FIG. 2. Probe-specific enrichment was determined by a SR >5 in HHS-475 probe/DMSO comparison. Liganded tyrosine sites were identified by SR >5 in proteomes from ligand-treated cells (HHS-0101, -0201, -0301, -0401, and -0701). The proteins and tyrosine sites from the heatmap are listed in Table 2. At the bottom of the figure is a zoomed view of prostaglandin reductase 2 (PTGR2) liganded site from the main heatmap above with scale bar shown on right. FIG. 3B is a graph showing the distribution of liganded proteins found with a DrugBank match (All DBP, ˜35%) compared with proteins without a database match (Non DBP, ˜65%). FIG. 3C is a graph showing domain enrichment distribution of probe-competed proteins (SR>5; Q-value cut-off of 0.05 following a Benjamini-Hochberg correction (Q-value cut-off of 0.05) using a two-sided binomial test criteria). All data shown are representative of n=3 biological replicates.

FIGS. 4A-4C: Exemplary sulfonyl-triazole exchange (SuTEx) ligand HHS-0401 functions as a cell-active ligand of fumarylacetoacetase (FAH) tyrosine 244 (Y244). FIG. 4A is a series of first mass spectrometer (MS1) extracted ion chromatograms (EIC) used to determine specific enrichment (probe/no probe) of the FAH Y244 site by quantitative chemical proteomics with SuTEx probe HHS-475. The FAH Y244 site is liganded (>90%) by HHS-0401 in live human sarcomatoid mesothelioma (DM93) cells treated with ligand. Equivalent mixing of light and heavy DM93 proteomes is verified by a stable isotope labeling by amino acids in cell culture (SILAC) ratio (SR) of ˜1 in probe/probe control samples. FAH amino acid subsequence WEYYPLGPFLGK (SEQ ID NO: 1) containing FAH Y244 is shown at the top. Data shown is representative of n=3 biological replicates. FIG. 4B is an image of gel-based activity-based protein profiling (ABPP) analysis of human embryonic kidney (HEK293T) cells expressing recombinant FAH treated with vehicle (DMSO) or SuTEx ligand (25 micromolar (μM), 2 hours, 37° C.) followed by SuTEx probe HHS-475 labeling (100 μM, 2 hours, 37° C.) used to verify the liquid chromatography-mass spectrometry (LC-MS) results shown in FIG. 4A. The FAH Y244F mutant protein was unable to be recombinantly expressed, and thus was not labelled by probe. FIG. 4C is an image of gel-based activity-based protein profiling (ABPP) analysis showing concentration-dependent blockade of probe labeling of recombinant FAH in HHS-0401-treated cells (2 hours). Equivalent expression of recombinant FAH was confirmed by western blots with anti-FLAG antibody. Stable isotope labeling by amino acids in cell culture (SILAC) ratio (SR) for representative MS1 EICs are shown.

FIGS. 5A-5C: Exemplary sulfonyl-triazole exchange (SuTEx) ligand HHS-0701 is a tyrosine-reactive ligand of prostaglandin reductase 2 (PTGR2) tyrosine 100 (Y100). FIG. 5A is a series of first mass spectrometer (MS1) extracted ion chromatograms (EIC) indicating that PTGR2 Y100 is liganded by HHS-0701 (˜90%) but not by a structurally analogous control compound HHS-0101 in ligand-treated human sarcomatoid mesothelioma (DM93) cells. Specific enrichment of the PTGR2 Y100 site and equivalent mixing of light and heavy proteomes was verified by the probe/no probe (SILAC ratio (SR) >5) and probe/probe (SR ˜1) control samples, respectively. PTGR2 amino acid subsequence GDFVTSFYWPWQTK (SEQ ID NO:2) containing PTGR2 Y100 is shown at the top. Data shown is representative of n=3 biological replicates. FIG. 5B is an image of gel-based activity-based protein profiling (ABPP) analysis of human embryonic kidney (HEK293T) cells expressing recombinant PTGR2 confirming liquid chromatography-mass spectrometry (LC-MS) findings that HHS-0701, but not HHS-0101, could block HHS-475 labeling of PTGR2 in live cells (25 micromolar (μM), 2 hours, 37° C.). The HHS-475 probe labeling of PTGR2 Y100F mutant protein was observed to be reduced compared with wild-type (WT) counterpart (n=2 replicates). FIG. 5C is an image of gel-based ABPP analysis showing concentration-dependent blockade of recombinant PTGR2 in HHS-0701-treated cells (2 hours). Western blots with anti-FLAG antibody were used to confirm equivalent expression of recombinant PTGR2. Stable isotope labeling by amino acids in cell culture (SILAC) ratio (SR) for representative MS1 EICs are shown.

FIGS. 6A-6B: Exemplary sulfonyl-triazole exchange (SuTEx) ligand HHS-0701 functions as an inhibitor of prostaglandin reductase 2 (PTGR2) biochemical activity. FIG. 6A is a schematic diagram showing a biochemical assay for measuring PTGR2 catalytic activity. PTGR2 catalyzes NADPH-dependent reduction of 15-keto-prostaglandin E₂ (15-keto-PGE₂) to 13,14-dihydro-15-keto-prostaglandin E₂ (13,14-dihydro-15-keto-PGE₂). FIG. 6B is a graph showing that recombinant PTGR2-HEK293T proteomes significantly increased metabolism of 15-keto-PGE₂ substrate compared with non-overexpressing mock HEK293T proteomes. Treatment of recombinant PTGR2-HEK293T cells with HHS-0701 resulted in concentration-dependent blockade of PTGR2 biochemical activity. In contrast, cellular treatment with the negative control compound HHS-0101 at the highest concentration (25 micromolar (μM)) had negligible effects on PTGR2 biochemical activity. Statistical significance was determined using a Dunnett multiple comparison correction following a one-way ANOVA test (*P<0.05, **P<0.01). All data shown represent mean s.e.m.; n=3 biological samples.

FIG. 7 is a graph showing prostaglandin reductase 2 (PTGR2) substrate assay data for wild-type (WT), Y100F mutant, and Y265F mutant PTGR2. Data shown for n=2 independent biological replicates.

FIGS. 8A-8B: Prostaglandin reductase 2 (PTGR2) inhibitors in live cells. FIG. 8A a schematic drawing showing the chemical structures of exemplary sulfonyl-triazole exchange (SuTEx) ligands that inhibit PTGR2. FIG. 8B is a composite image of gel-based proteomics analysis supporting inhibition of PTGR2 by exemplary SuTEx ligands, e.g., AMC-0703.

FIGS. 9A-9D: In vitro competition studies with recombinant prostaglandin reductase 2 (PTGR2). FIG. 9A is an image of in vitro gel-based chemical proteomics assay using sulfonyl-triazole exchange (SuTEx) probe RJS-1027 (1 micromolar (μM)) as a probe to measure competition of binding PTGR2 with SuTEx inhibitors (0.5 μM). Anti-FLAG and glyceraldehyde 3-phosphate dehydrogenase (GAPDH) western blots are shown below the long gel and were performed separately. FIG. 9B is a graph showing quantitative comparison of inhibitor competition with probe labeling normalized to the negative control HHS-0101. FIG. 9C is an image of an in vitro gel-based chemical proteomics assay using RJS-1027 (1 μM) as a probe to measure competition of binding PTGR2 with potent inhibitors, e.g., AMC-0703 (0.5 μM) and RJG-1189 (0.5 μM), and a dose response with RJG-2036. FIG. 9D is a graph showing quantitative comparison of RJG-2036 competition with probe labeling normalized to the negative control HHS-0101.

FIGS. 10A-10C: In situ competition studies with recombinant prostaglandin reductase 2 (PGTR2). FIG. 10A is a schematic diagram showing chemical structures of sulfonyl-triazole exchange (SuTEx) compounds used for in situ studies: RJS-1027 as the probe for reporting inhibitor labeling of PTGR2, HHS-0101 as the negative control for trace PTGR2 labeling, and RJG-2036 as an inhibitor of probe labeling. FIG. 10B is an image of an in situ gel-based chemical proteomics assay using RJS-1027 (1 micromolar (μM)) as a probe to measure competition of binding PTGR2 (see box) with RJG-2036 (dose response). Anti-FLAG and glyceraldehyde 3-phosphate dehydrogenase (GAPDH) western blots are shown below the long gel and were performed separately. FIG. 10C is a graph of the quantitation of RJG-2036 competition with probe labeling normalized to the wild-type, recombinant PTGR2 (WT) using RJS-1027 as a probe (50% inhibitory concentration (IC₅₀)=89.5 nanomolar (nM); data represents two biological replicates).

FIG. 11 is a graph showing the results of an enzymatic assay measuring the ability of wild-type, recombinant prostaglandin reductase 2 (PTGR2 (WT)) to reduce its natural substrate (15-keto-prostaglandin 2 (15-keto-PGE2) to 13,14-dihydro-15-keto-PGE2 in the absence (WT) or presence of inhibitor (500 nanomolar (nM) RJG-2036) compared to heat-inactivated PTGR2 (Heat Inactive) and Mock, which did not overexpress recombinant PTGR2 and thus contains negligible PTGR2.

### DETAILED DESCRIPTION

The presently disclosed subject matter relates to the use of sulfur-heterocycle exchange chemistry for investigating tyrosine and/or lysine reactivity, function and post-translational modification state in proteomes and live cells, as well as for use in preparing pharmaceuticals that target druggable tyrosines and/or lysines. For example, sulfonyl-triazoles have emerged as a new reactive group for covalent modification of tyrosine sites on proteins through sulfur-triazole exchange (SuTEx) chemistry. See PCT International Publication No. 2020/214336, the disclosure of which is incorporated by reference in its entirety. The presently disclosed subject matter relates, in one aspect, to the further development of this sulfur electrophile and related sulfur-heterocycles as ligands with cellular activity. In some embodiments, the presently disclosed subject matter relates to fragment-based ligand discovery in live cells to identify SuTEx compounds capable of liganding tyrosine sites on diverse protein targets. Quantitative chemical proteomic findings were verified by demonstrating concentration dependent activity of SuTEx ligands but not inactive counterparts against recombinant protein targets directly in live cells. The presently disclosed subject matter further provides structure-activity relationship studies identifying new tyrosine-active SuTEx ligands as cell-active inhibitors, e.g., capable of blocking prostaglandin reductase 2 (PTGR2) biochemical activity. Accordingly, in some embodiments, the presently disclosed subject matter relates to potent PTGR2 inhibitors, pharmaceutical compositions thereof, and to methods of inhibiting PTGR2.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

In some embodiments, the terms “fragment”, “segment”, or “subsequence” as used herein refers to a portion of an amino acid sequence, comprising at least one amino acid, or a portion of a nucleic acid sequence comprising at least one nucleotide. Thus, in some embodiments, the terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

In some embodiments, the term “fragment” refers to a compound (e.g., a small molecule compound) that can react with a reactive amino acid residue (e.g., a reactive tyrosine or a reactive lysine) to form an adduct comprising a modified amino acid (e.g., tyrosine or lysine) residue. Thus, in some embodiments, the terms “fragment” and “ligand” are used interchangeably. In some embodiments, the term “fragment” refers to that portion of a ligand that remains covalently attached to the reactive amino acid residue.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

The terms “high throughput protein identification,” “proteomics” and other related terms are used herein to refer to the processes of identification of a large number or (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which analysis, diagnosis, treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some embodiments, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some embodiments, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, cycloalkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, cycloalkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic.

The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂.

The term “carbonyl” refers to the —(C═O)— or a double bonded oxygen substituent attached to a carbon atom of a previously named parent group.

The term “carboxyl” refers to the —COOH group.

The terms “halo”, “halide”, or “halogen” as used herein refer to fluoro, chloro, bromo, and iodo groups.

The term “haloalkyl” can be used to refer to an alkyl group wherein one or more hydrogen atoms have been replaced by halo groups.

The term “perhaloalkyl” refers to an alkyl group wherein all of the hydrogen atoms are replaced by halo. Thus, for example, perhaloalkyl can refer to a “perfluroalkyl” group wherein all of the hydrogen atoms of the alkyl group are replaced by fluoro. Perhaloalkyl groups include, but are not limited to, —CF₃.

The terms “hydroxyl” and “hydroxy” refer to the —OH group.

The term “oxo” refers to a compound described previously herein wherein a carbon atom is replaced by an oxygen atom.

The term “cyano” refers to the —CN group.

The term “nitro” refers to the —NO₂ group.

### II. Ligands

Small molecules can serve as versatile tools for perturbing the functions of proteins in biological systems. Many human proteins currently lack selective chemical ligands; and there are several classes of proteins that are currently considered as undruggable. Covalent ligands (also referred to herein as “fragments”) offer a strategy to expand the landscape of proteins amenable to targeting by small molecules. In some instances, covalent ligands combine features of recognition and reactivity, thereby providing for the targeting of sites on proteins that are difficult to address by reversible binding interactions alone.

As noted hereinabove, sulfonyl-triazoles have emerged as a new class of reactive compounds for covalent modification of tyrosine and/or lysine sites on proteins through sulfur-triazole exchange (SuTEx) chemistry. See PCT International Publication No. 2020/214336, the disclosure of which is incorporated by reference in its entirety. For example, Schemes 1, below, shows the reaction of a SuTEx compound (e.g., a SuTEx ligand or a SuTEx probe) with a protein having a reactive tyrosine (Y) or lysine (K). The SuTEx compound comprises a sulfur electrophile, i.e., a sulfonyl group directed attached to a nitrogen atom of a nitrogen-containing heteroaryl group. The nitrogen-containing heteroaryl group acts as a leaving group in the reaction of the compound with the nucleophilic phenol or amine of the tyrosine or lysine, resulting in a modified protein where a modified tyrosine or lysine residue is covalently attached to the SuTEx compound sulfonyl group, which is itself directly attached to an adduct group (AG) or “fragment” from the original SuTEx compound. AGs of SuTEx ligands can include a variety of optionally substituted alkyl, cycloalkyl, heterocyclic, aryl, aralkyl, and heteroaryl groups, while SuTEx “probes” can contain AG groups that comprise an alkyne group, a fluorophore moiety, a detectable moiety, or a combination thereof. While the nitrogen-containing heteroaryl group shown in the SuTEx compound of Scheme 1 is a 1,2,4-triazole or a 1,2,3-triazole substituted by an R group (i.e., H or an aryl group substituent), SuTEx compounds can also include other nitrogen-containing heteroaryl groups as the leaving group, e.g., pyrazole, imidazole, or tetrazole, each of which can be optionally substituted by one or more aryl group substituents.

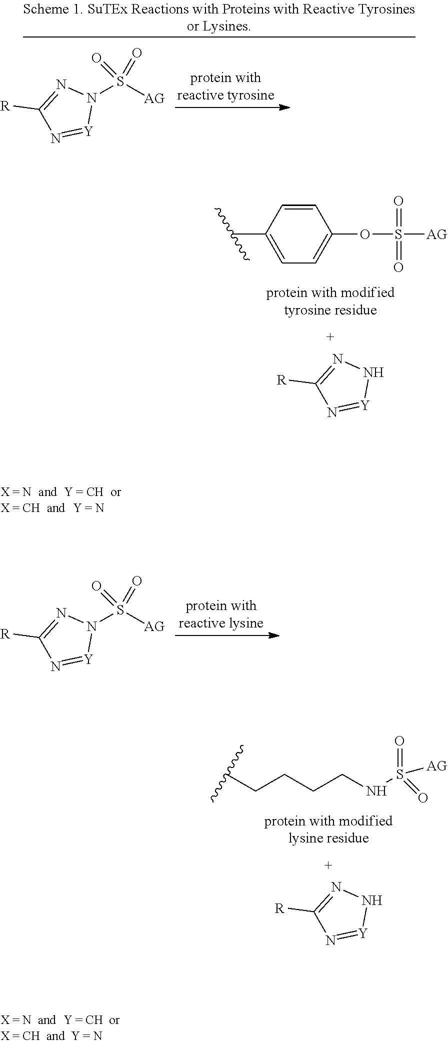

In some embodiments, a ligand of the presently disclosed subject matter can compete with a probe compound described herein for binding with a reactive tyrosine and/or lysine residue. In some embodiments, the ligand molecule comprises a fragment moiety that facilitates interaction of the compound with a reactive tyrosine and/or lysine residue. In some cases, the ligand comprises a fragment moiety that facilitates hydrophobic interaction, hydrogen bonding, or a combination thereof. The presently disclosed ligands are typically non-naturally occurring and/or form non-naturally occurring products after reaction with the phenol group of a tyrosine residue of a tyrosine-containing protein or an amino group of a lysine residue of a lysine containing protein.

The presently disclosed subject matter relates, in one aspect, to the further development of SuTEx ligands. In some embodiments, the presently disclosed subject matter provides a compound (e.g., a tyrosine-reactive and/or lysine-reactive ligand compound) having a structure of Formula (I):

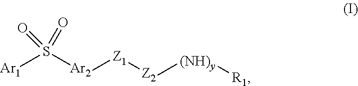

wherein: y is 0 or 1; Ar₁ is triazole, substituted triazole, imidazole, substituted imidazole, pyrazole, substituted pyrazole, tetrazole, or substituted tetrazole; Ar₂ is aryl or heteroaryl; Z₁ is —CH₂— or —C(═O)—; Z₂ is a heterocyclic, heteroaryl, substituted heterocyclic or substituted heteroaryl group; and R₁ is aryl, substituted aryl, heteroaryl or substituted heteroaryl; subject to the proviso that when Z₂ is piperidinyl, y is 1 and when Z₁ is —C(═O)— and Z₂ is piperazinyl, R₁ is not substituted pyrimidinyl; or a pharmaceutically acceptable salt thereof. In general, the compound of Formula (I) is a compound wherein a nitrogen atom of the Ar₁ group is directly attached to the sulfur atom of the sulfonyl group. In some embodiments, when Ar₂ is heteroaryl, the sulfur atom of the sulfonyl group is not directly attached to a heteroatom in Ar₂.

In some embodiments, Ar₁ is triazole or substituted triazole. In some embodiments Ar₁ is 1,2,4-triazole or substituted 1,2,4-triazole. In some embodiments, Ar₁ is 1,2,3-triazole or substituted 1,2,3-triazole.

In some embodiments, Ar₂ is an aryl or heteroaryl group comprising a six-membered ring. In some embodiments, Ar₂ is phenyl or a nitrogen-containing heteroaryl group wherein a nitrogen atom of the nitrogen-containing heteroaryl group is not directly attached to the sulfur atom of the sulfonyl group in the structure of Formula (I). Thus, for example, the ligand of Formula (I) can have the structure:

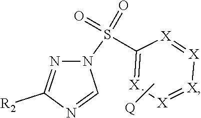

wherein each X is selected from N and CH, R₂ is H or an aryl group substituent, and Q is —Z₁-Z₂—(NH)_{y}—R₁, wherein Z₁, Z₂, y, and R₁ are as defined for Formula (I). In some embodiments, 0, 1, 2, or 3 X groups can be N. In some embodiments, Ar₂ is phenyl, pyridinyl, pyrimidinyl, or triazinyl. Scheme 2, below, shows exemplary ligand structures where Ar₂ is a nitrogen-containing, six-membered heteroaryl group.

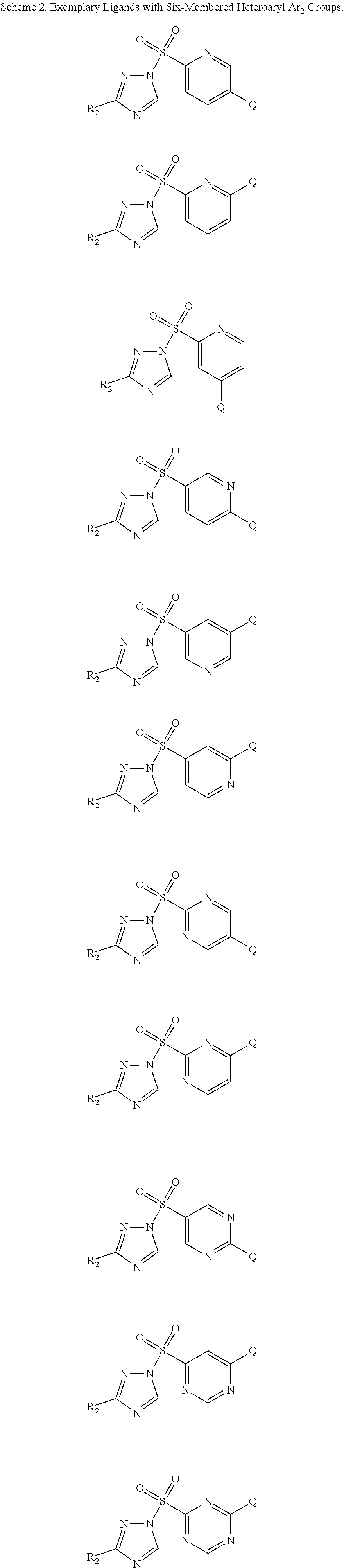

In some embodiments, Ar₂ is a heteroaryl group comprising a five-membered ring. Thus, in some embodiments, the compound of Formula (I) can have the structure:

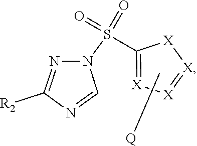

wherein each X is selected from N, NR, O, S, and CH, subject to the proviso that at least one X is not CH; R₂ is H or an aryl group substituent; and Q is —Z₁-Z₂—(NH)_{y}—R₁, wherein Z₁, Z₂, y, and R₁ are as defined for Formula (I), and R is H, alkyl, aralkyl or aryl. In some embodiments, 0, 1, 2, or 3 X groups can be N. In some embodiments, Ar₂ is selected from furanyl, triazole (e.g., 1,2,4-triazole), thiazole, oxazole, isoxazole, and 1,2,4-oxadiazole. Scheme 3, below, shows exemplary ligand structures where Ar₂ is a five-membered heteroaryl group.

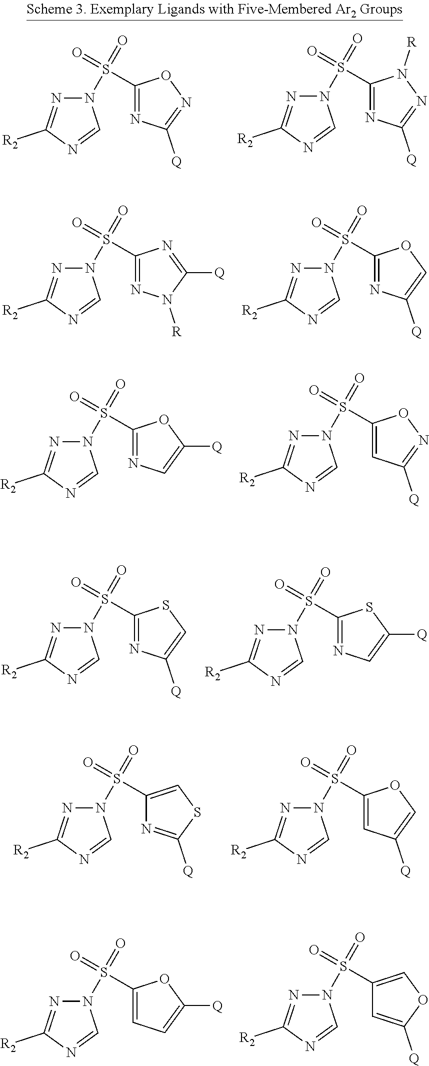

Exemplary Q groups where Z₁ is —C(═O)— are shown in Scheme 4, below. R₁ and y in the compounds in Scheme 4 are as defined for Formula (I), while R″ is alkyl, substituted alkyl, or aryl. Scheme 5, below, shows exemplary Q groups comprising the same Z₂ groups when Z₁ is —CH₂—. See Scheme 5, below.

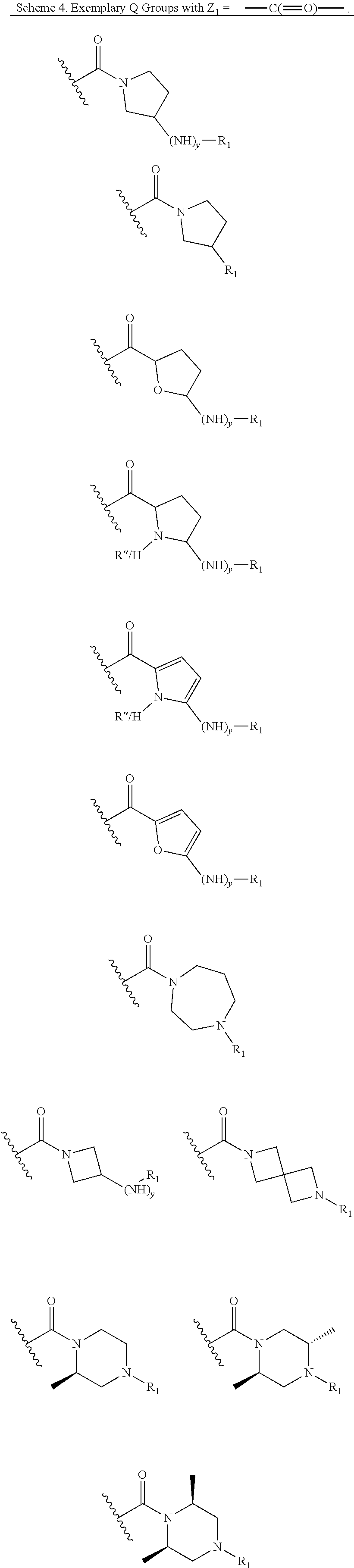

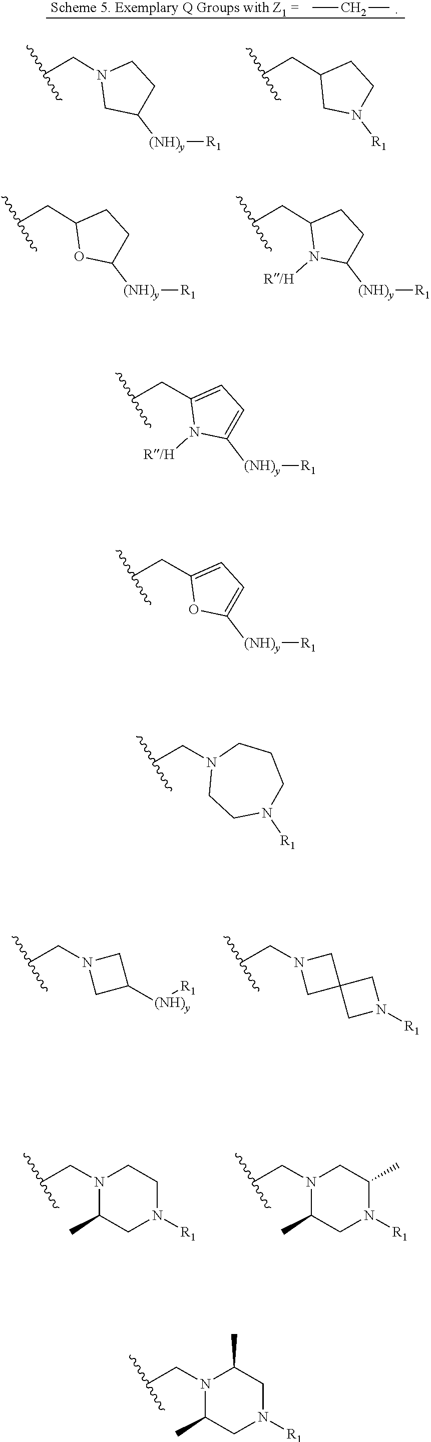

In some embodiments, Z₂ is selected from the group including, but not limited to, piperidinyl, piperazinyl, tetrahydrofuranyl, pyrrolidinyl, pyrrolyl, furanyl, diazepanyl, azetidinyl, and 2,6-diaza[3.3]heptanyl.

In some embodiments, R₁ is an optionally substituted aryl or heteroaryl group, selected from the group including, but not limited to, phenyl, pyridyl, indolinyl, tetrahydroquinolinyl, indolyl, benzofuranyl, indanyl, dihydrobenzofuranyl, chromanyl, benzofuranyl, anthranilyl, benzofurazanyl, isoindolyl, oxindolyl, and isocarbostyrilyl, each of which can be substituted by one or more aryl group substituents Some exemplary R₁ groups of the presently disclosed ligands are shown in Scheme 6, below.

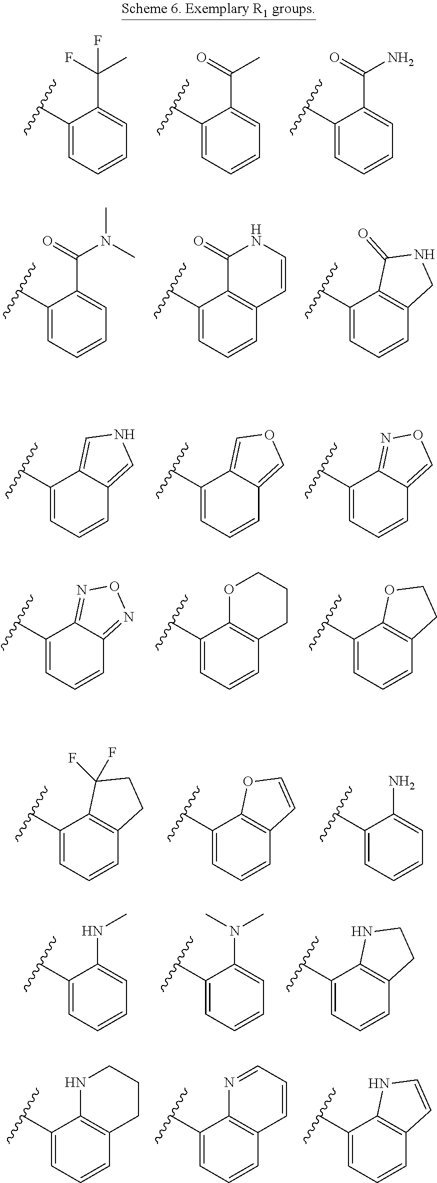

In some embodiments, Z₂ is selected from piperazinyl and piperidinyl. In some embodiments, the compound having a structure of Formula (I) has a structure of Formula (II):

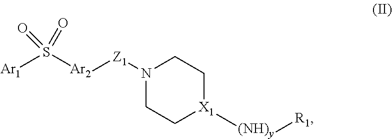

wherein: y is 0 or 1; Ar₁ is selected from the group comprising triazole, substituted triazole, imidazole, substituted imidazole, pyrazole, substituted pyrazole, tetrazole, and substituted tetrazole; Ar₂ is aryl or heteroaryl; Z₁ is —CH₂— or —C(═O)—; X₁ is N or CH; and R₁ is aryl, substituted aryl, heteroaryl or substituted heteroaryl; subject to the proviso that when X₁ is CH, y is 1 and when X₁ is N, y is 0; or a pharmaceutically acceptable salt thereof.

In some embodiments, Z₁ is —C(═O)— (i.e., carbonyl). In some embodiments Z₁ is —CH₂— (i.e., methylene).

In some embodiments, the compound having a structure of Formula (I) has a structure of Formula (III):

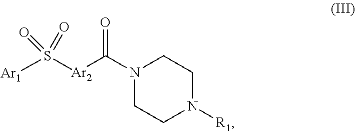

wherein: Ar₁ is selected from the group comprising triazole, substituted triazole, imidazole, substituted imidazole, pyrazole, substituted pyrazole, tetrazole, and substituted tetrazole; Ar₂ is aryl or heteroaryl; and R₁ is aryl, heteroaryl, substituted aryl, or substituted heteroaryl; or a pharmaceutically acceptable salt thereof. In some embodiments, R₁ is selected from phenyl, substituted phenyl, pyridyl, and substituted pyridyl.

In some embodiments, Ar₂ is phenyl. In some embodiments, Ar₂ is phenyl and the compound of Formula (III) has a structure of Formula (IIIa) or Formula (IIIb):

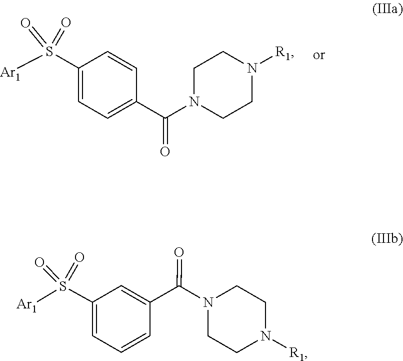

wherein: Ar₁ is selected from the group comprising triazole, substituted triazole, imidazole, substituted imidazole, pyrazole, substituted pyrazole, tetrazole, and substituted tetrazole; and R₁ is aryl, heteroaryl, substituted aryl, or substituted heteroaryl; or a pharmaceutically acceptable salt thereof. In some embodiments, R₁ is phenyl, substituted phenyl, pyridyl, or substituted pyridyl.

In some embodiments, Ar₁ is triazole or substituted triazole. In some embodiments, Ar₁ is 1,2,4-triazole, 1,2,3-triazole, substituted 1,2,4-triazole, or substituted 1,2,3-triazole.

In some embodiments, Ar₁ is a 1,2,4-triazole or substituted 1,2,4-triazole group and the compound of Formula (III) has a structure of Formula (IV):

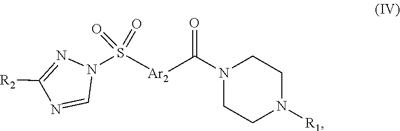

wherein: Ar₂ is aryl or heteroaryl; R₁ is aryl, substituted aryl, heteroaryl, or substituted heteroaryl; and R₂ is an aryl group substituent (e.g., selected from H, alkyl, cycloalkyl, aryl, and substituted aryl); or a pharmaceutically acceptable salt thereof. In some embodiments, R₁ is selected from phenyl, substituted phenyl, pyridyl, or substituted pyridyl.

In some embodiments, Ar₂ is phenyl and the compound of Formula (IV) has a structure of Formula (IVa) or (IVb):

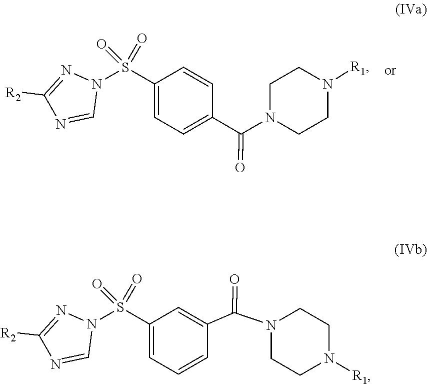

wherein: R₁ is phenyl, substituted phenyl, pyridyl, or substituted pyridyl; and R₂ is selected from H, alkyl, cycloalkyl, aryl, and substituted aryl; or a pharmaceutically acceptable salt thereof.

In some embodiments, R₂ is selected from H, cycloalkyl, phenyl, furanyl, pyridyl, and substituted phenyl. For example, in some embodiments, R₂ is substituted phenyl. In some embodiments, the substituted phenyl is phenyl substituted with one or more substituent selected from the group comprising halo (e.g., Cl, Br, F, or I), perfluoroalkyl (e.g., —CF₃), alkoxy (e.g., C1-C6 alkoxy, such as methoxy, ethoxy, n-propoxy, isopropoxy, n-butoxy, tert-butoxy, etc.), perfluoralkoxy (e.g., CF₃O— or CF₃CF₂O—) and aryl. In some embodiments, R₂ is selected from H, 2-pyridyl, and 2-methoxyphenyl.

In some embodiments, R₁ is substituted phenyl or substituted pyridyl, wherein said substituted phenyl or substituted pyridyl are phenyl or pyridyl substituted with one or more substituent selected from the group consisting of alkyl (e.g., C1-C6 alkyl), halo (i.e., F, Cl, Br, or I), haloalkyl (e.g., fluoroalkyl), alkoxy (e.g., C1-C6 alkoxy), acyl (e.g., —C(═O)CH₃), —C(═O)—NH₂, amino (e.g., —NH₂), alkylamino (e.g., —NHCH₃), and dialkylamino (e.g., —N(CH₃)₂ or —N(CH₂CH₃)₂). In some embodiments, R₁ is phenyl or pyridyl substituted by one or more substituents selected from alkyl, halo, and alkoxy.

In some embodiments, R₁ is substituted phenyl. In some embodiments, R₁ is phenyl substituted by one or more (e.g., 1, 2, 3, 4, or 5) substituents selected from halo and alkoxy. In some embodiments, R₁ is alkoxy-substituted phenyl. In some embodiments, R₁ is, methoxy-substituted phenyl (e.g., 2-methoxyphenyl, 3-methoxyphenyl, or 4-methoxyphenyl). In some embodiments, R₁ is 2-methoxyphenyl.

In some embodiments, the compound is selected from the group comprising:
(4-((1H-1,2,4-triazol-1-yl)sulfonyl)phenyl)(4-phenylpiperazin-1-yl)methanone (AMC-0702);
(4-((1H-1,2,4-triazol-1-yl)sulfonyl)phenyl)(4-(2-methoxyphenyl)piperazin-1-yl)methanone (AMC-0703);
(4-(2-methoxyphenyl)piperazin-1-yl)(4-((3-phenyl-1H-1,2,4-triazol-1-yl)sulfonyl)-phenyl)methanone (RJG-1101);
(4-((1H-1,2,4-triazol-1-yl)sulfonyl)phenyl)(4-(3-methoxyphenyl)piperazin-1-yl)-methanone (RJG-1103);
(4-((1H-1,2,4-triazol-1-yl)sulfonyl)phenyl)(4-(4-methoxyphenyl)piperazin-1-yl)-methanone (RJG-1105);
(4-((3-(4-bromophenyl)-1H-1,2,4-triazol-1-yl)sulfonyl)phenyl)(4-(2-methoxy-phenyl)piperazin-1-yl)methanone (RJG-1112);
(4-((3-(4-fluorophenyl)-1H-1,2,4-triazol-1-yl)sulfonyl)phenyl)(4-(2-methoxy-phenyl)piperazin-1-yl)methanone (RJG-1114);
(4-(2-methoxyphenyl)piperazin-1-yl)(4-((3-(4-(trifluoromethyl)phenyl)-1H-1,2,4-triazol-1-yl)sulfonyl)phenyl)methanone (RJG-1115);
(4-((3-(furan-2-yl)-1H-1,2,4-triazol-1-yl)sulfonyl)phenyl)(4-(2-methoxyphenyl)-piperazin-1-yl)methanone RJG-1185,
(4-((3-(2-fluorophenyl)-1H-1,2,4-triazol-1-yl)sulfonyl)phenyl)(4-(2-methoxy-phenyl)piperazin-1-yl)methanone (RJG-1187);
(4-((3-(3-fluorophenyl)-1H-1,2,4-triazol-1-yl)sulfonyl)phenyl)(4-(2-methoxy-phenyl)piperazin-1-yl)methanone (RJG-1188);
(4-(2-methoxyphenyl)piperazin-1-yl)(4-((3-(pyridin-3-yl)-1H-1,2,4-triazol-1-yl)-sulfonyl)phenyl)methanone (RJG-1189);
(4-(2-methoxyphenyl)piperazin-1-yl)(4-((3-(4-(trifluoromethoxy)phenyl)-1H-1,2,4-triazol-1-yl)sulfonyl)phenyl)methanone (RJG-1228);
(4-(4-iodophenyl)piperazin-1-yl)(4-((3-phenyl-1H-1,2,4-triazol-1-yl)sulfonyl)-phenyl)methanone (RJG-1257);
(4-((1H-1,2,4-triazol-1-yl)sulfonyl)phenyl)(4-(pyridin-2-yl)piperazin-1-yl)-methanone (RJG-1285);
(4-(2-methoxyphenyl)piperazin-1-yl)(4-((3-(pyridin-4-yl)-1H-1,2,4-triazol-1-yl)-sulfonyl)phenyl)methanone (RJG-1291);
(4-((3-([1,1′-biphenyl]-4-yl)-1H-1,2,4-triazol-1-yl)sulfonyl)phenyl)(4-(2-methoxy-phenyl)piperazin-1-yl)methanone (RJG-2011);
(3-((1H-1,2,4-triazol-1-yl)sulfonyl)phenyl)(4-(2-methoxyphenyl)piperazin-1-yl)-methanone (RJG-2036);
(4-((3-cyclopropyl-1H-1,2,4-triazol-1-yl)sulfonyl)phenyl)(4-(2-methoxyphenyl)-piperazin-1-yl)methanone (RJG-2048);
4-((3-cyclobutyl-1H-1,2,4-triazol-1-yl)sulfonyl)phenyl)(4-(2-methoxyphenyl)-piperazin-1-yl)methanone (RJG-2049);
(4-((3-cyclopentyl-1H-1,2,4-triazol-1-yl)sulfonyl)phenyl)(4-(2-methoxyphenyl)-piperazin-1-yl)methanone (RJG-2050);
(4-((3-cyclohexyl-1H-1,2,4-triazol-1-yl)sulfonyl)phenyl)(4-(2-methoxyphenyl)-piperazin-1-yl)methanone (RJG-2051);
(4-((3-(4-methoxyphenyl)-1H-1,2,4-triazol-1-yl)sulfonyl)phenyl)(4-(2-methoxy-phenyl)piperazin-1-yl)methanone (RJG-2056); and
(4-((1H-1,2,4-triazol-1-yl)sulfonyl)phenyl)(4-((2-methoxyphenyl)amino)piperidin-1-yl)methanone (RJG-2058) or a pharmaceutically acceptable salt thereof.

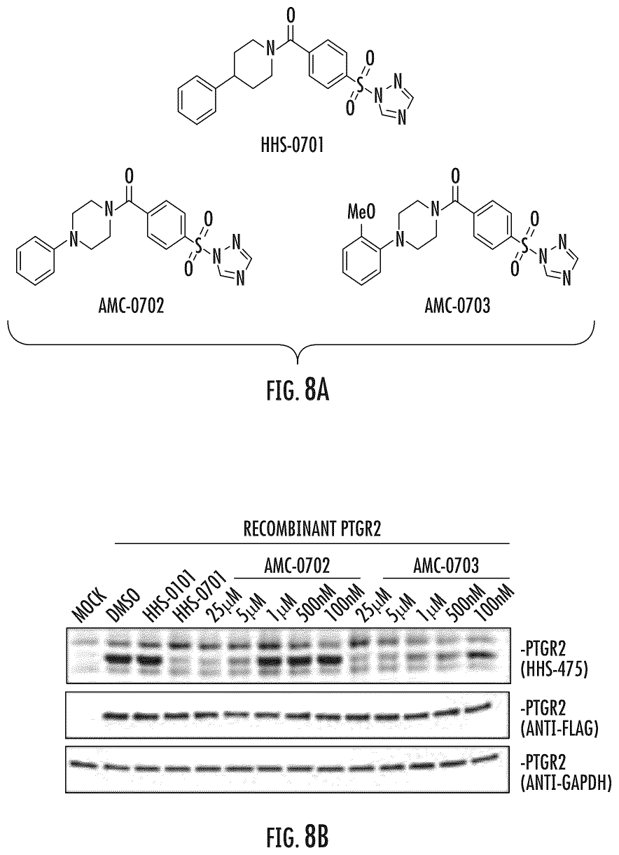

Structures of these compounds are shown below in Example 8 and in FIG. 8A. In some embodiments, the compound is selected from (4-((1H-1,2,4-triazol-1-yl)sulfonyl)phenyl)(4-(2-methoxyphenyl)piperazin-1-yl)methanone (AMC-0703), (4-(2-methoxyphenyl)piperazin-1-yl)(4-((3-(pyridin-3-yl)-1H-1,2,4-triazol-1-yl)-sulfonyl)-phenyl)methanone (RJG-1189), and (3-((1H-1,2,4-triazol-1-yl)sulfonyl)phenyl)-(4-(2-methoxyphenyl)piperazin-1-yl)-methanone (RJG-2036); or a pharmaceutically acceptable salt thereof. In some embodiments, the compound is (4-((1H-1,2,4-triazol-1-yl)sulfonyl)phenyl)(4-(2-methoxyphenyl)piperazin-1-yl)methanone (AMC-0703) or a pharmaceutically acceptable salt thereof. In some embodiments, the compound is (4-(2-methoxyphenyl)piperazin-1-yl)(4-((3-(pyridin-3-yl)-1H-1,2,4-triazol-1-yl)-sulfonyl)-phenyl)methanone (RJG-1189) or a pharmaceutically acceptable salt thereof. In some embodiments, the compound is (3-((1H-1,2,4-triazol-1-yl)sulfonyl)phenyl)-(4-(2-methoxyphenyl)piperazin-1-yl)-methanone (RJG-2036) or a pharmaceutically acceptable salt thereof.

In some embodiments, the compound can be a compound of Formula (I) where Z₂ is not present, y is 1, and R₁ is substituted aryl or substituted heteroaryl. In some embodiments, R₁ is an aryl or heteroaryl group further substituted by an aryl, heteroaryl, substituted aryl, or substituted heteroaryl. In some embodiments, the compound has a structure of Formula (I′)

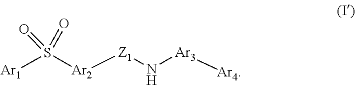

wherein Ar₁ is selected from the group comprising triazole, substituted triazole, imidazole, substituted imidazole, pyrazole, substituted pyrazole, tetrazole, and substituted tetrazole; Ar₂ is aryl or heteroaryl; Z₁ is —CH₂— or —C(═O)—; Ar₃ is aryl or heteroaryl; and Ar₄ is substituted aryl, heteroaryl, or substituted heteroaryl; or a pharmaceutically acceptable salt thereof.

In some embodiments, Ar₁ is triazole or substituted triazole. In some embodiments, Ar₁ is 1,2,4-triazole or substituted 1,2,4-triazole. In some embodiments, Ar₁ is 1,2,3-triazole or substituted 1,2,3-triazole.

In some embodiments, Z₁ is —C(═O)—.

In some embodiments, Ar₃ is phenyl or pyridyl. In some embodiments, Ar₃ is phenyl.

In some embodiments, Ar₄ is substituted phenyl. In some embodiments, Ar₄ is phenyl substituted by alkoxy or perfluoroalkoxy. In some embodiments, Ar₄ is 2-methoxyphenyl or 2-trifluoromethoxyphenyl. In some embodiments, Ar₄ is pyridyl.

In some embodiments, the compound of Formula (I′) is selected from the group comprising:

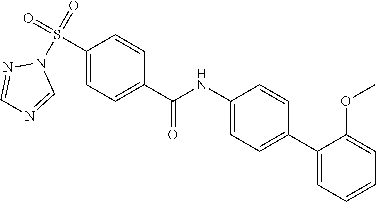 [SMILES: COc1ccccc1-c1ccc(NC(=O)c2ccc(S(=O)(=O)n3cncn3)cc2)cc1]

(i.e., 4-((1H-1,2,4-triazol-1-yl)sulfonyl)-N-(2′-methoxy-[1,1′-biphenyl]-4-yl)benzamide (RJG-2040A)),

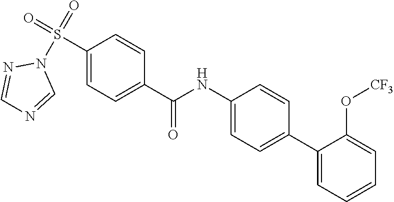 [SMILES: O=C(Nc1ccc(-c2ccccc2OC(F)(F)F)cc1)c1ccc(S(=O)(=O)n2cncn2)cc1]

(i.e., 4-((1H-1,2,4-triazol-1-yl)sulfonyl)-N-(2′-(trifluoromethoxy)-[1,1′-biphenyl]-4-yl)benzamide (RJG-2040B)), and

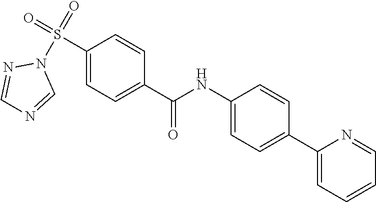 [SMILES: O=C(Nc1ccc(-c2ccccn2)cc1)c1ccc(S(=O)(=O)n2cncn2)cc1]

(i.e., 4-((1H-1,2,4-triazol-1-yl)sulfonyl)-N-(4-(pyridin-2-yl)phenyl)benzamide (RJG-2040C)); or a pharmaceutically acceptable salt thereof.

As noted above, in some embodiments, the presently disclosed compounds can be provided as a pharmaceutically acceptable salt. As used herein, the term “physiologically acceptable salt” means a salt form of the recited compound which is compatible with any other ingredients of a pharmaceutical composition and/or which is not deleterious to a subject to which the composition is to be administered (e.g., a human or other mammalian subject).

Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethyl ammonium, methylamine, dimethylamine, trimethylamine, ethylamine, basic amino acids, e. g., lysine and arginine dicyclohexylamine and the like.

Examples of metal salts include lithium, sodium, potassium, and magnesium salts and the like. Examples of ammonium and alkylated ammonium salts include ammonium, methylammonium, dimethylammonium, trimethylammonium, ethylammonium, hydroxyethylammonium, di ethyl ammonium, butylammonium, tetramethylammonium salts and the like.

In some embodiments, the presently disclosed compounds can further be provided as a solvate.

In some embodiments, the presently disclosed subject matter encompasses the preparation and use of pharmaceutical compositions comprising a ligand compound as described herein. The pharmaceutical compositions can be useful for treatment of diseases and disorders as would be apparent upon review of the instant disclosure as an active ingredient. Such a pharmaceutical composition can comprise, consist essentially of, or consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art. Thus, in some embodiments, the presently disclosed subject matter provides a pharmaceutical composition comprising (a) a compound of Formula (I), (II), (III), (IIIa), (Tub), (IV), (IVa), (IVb) or (I′), or a pharmaceutical salt thereof and (b) a pharmaceutically acceptable carrier.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peni- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pennsylvania, United States of America and/or Gennaro (ed.) (2003) Remington: The Science and Practice of Pharmacy, 20th edition Lippincott, Williams & Wilkins, Philadelphia, Pennsylvania, United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Other approaches include but are not limited to nanosizing the composition comprising a ligand compound as described herein to be delivered as a nanoparticle intravenously, intraperitoneal injection, or implanted beads with time release of a ligand compound as described herein.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

### III. Probes

In some embodiments, the presently disclosed subject matter provides a probe compound (e.g., a small molecule probe compound) that comprises a reactive moiety (i.e., a reactive electrophilic moiety) which can interact with the phenol group of a tyrosine residue of a tyrosine-containing protein and/or a nucleophilic group of the side chain of another amino acid residue, such as the primary amino group of a lysine residue of a lysine-containing protein. In some instances, the probe reacts with a tyrosine and/or lysine residue to form a covalent bond. Typically, the probe is a non-naturally occurring molecule, or forms a non-naturally occurring product (i.e., a “modified” protein or adduct) after reaction with the phenol group of a tyrosine residue of a tyrosine containing protein or other nucleophilic group of an amino acid, e.g., the primary amino group of a lysine residue. In some instances, the phenol group of a reactive tyrosine in the tyrosine-containing protein is connected to the small molecule fragment moiety via an —O—S(═O)₂— bond. In some instances, the primary amino group of a reactive lysine in a lysine-containing protein is connected to a small molecule fragment moiety via an —NH—S(═O)₂— bond.

For example, in some embodiments, the presently disclosed subject matter provides a probe compound that has a structure of Formula:

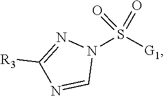

wherein: G₁ is a monovalent moiety comprising an alkyne moiety, a fluorophore moiety, a detectable labeling group, or a combination thereof, and R₃ is cycloalkyl, e.g., selected from cyclopropyl, cyclobutyl, cyclopentyl, and cyclohexyl. Thus, in some embodiments, the probe compound of can form a protein or peptide comprising at least one modified reactive tyrosine residue, wherein the modified reactive tyrosine comprises a structure:

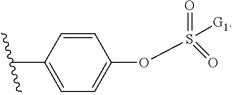

In some embodiments, the probe compound can form a protein or peptide comprising at least one modified reactive lysine residue, wherein the modified reactive lysine residue comprises a structure:

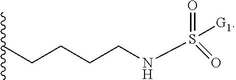

The fluorophore of G₁ can be any suitable fluorophore. In some embodiments, the fluorophore is selected from the group including, but not limited to, rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, and

In some embodiments, G₁ comprises a fluorophore moiety. In some cases, G₁ is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, the detectable labeling moiety is selected from the group comprising a member of a specific binding pair (e.g., biotin:streptavidin, antigen-antibody, nucleic acid:nucleic acid), a bead, a resin, a solid support, or a combination thereof. In some embodiments, the detectable labeling group is a biotin moiety, a streptavidin moiety, bead, resin, a solid support, or a combination thereof. In some embodiments, the detectable labeling moiety comprises biotin or a derivative thereof (e.g., desthiobiotin). In some embodiments, the detectable labeling moiety comprises a heavy isotope (i.e., ¹³C).

In some embodiments, Gi comprises an aryl group (e.g., Ar₂ as defined hereinabove for Formula (I)) directly attached to the sulfur atom of the sulfonyl group. Thus, in some embodiments, G₁ has a structure —Ar₂-G₃, wherein Ar₂ is aryl and G₃ is a monovalent moiety comprising an alkyne moiety, a fluorophore moiety, a detectable labeling group, or a combination thereof. In some embodiments, Ar₂ is selected from the group comprising phenyl, naphthyl, and pyridyl. In some embodiments, Ar₂ is phenyl. In some embodiments, G₃ comprises or consists of —C≡CH, -alkylene-C≡CH, —O-alkylene-C≡CH (e.g., —O—CH₂—C≡CH), or —C(═O)—NH-alkylene-C≡CH (e.g., C(═O)—NH—CH₂—C≡CH). In some embodiments, the alkylene group is a C₁-C₅ alkylene group. In some embodiments, the alkylene group is methylene.

In some embodiments, the probe compound has a structure of Formula (V):

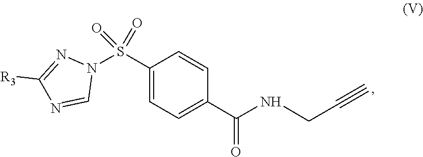

wherein R₃ is selected from cyclopropyl, cyclobutyl, cyclopentyl, and cyclohexyl. Thus, in some embodiments, the compound of Formula (V) is selected from the group comprising: 4-((3-cyclopropyl-1H-1,2,4-triazol-1-yl)sulfonyl)-N-(prop-2-yn-1-yl)benzamide (RJG-2043), 4-((3-cyclobutyl-1H-1,2,4-triazol-1-yl)sulfonyl)-N-(prop-2-yn-1-yl)benzamide (RJS-1027), 4-((3-cyclopentyl-1H-1,2,4-triazol-1-yl)sulfonyl)-N-(prop-2-yn-1-yl)benz-amide (RJG-2044), and 4-((3-cyclohexyl-1H-1,2,4-triazol-1-yl)sulfonyl)-N-(prop-2-yn-1-yl)benzamide (RJG-2045).

### IV. Synthesis

The probes and ligands of the presently disclosed subject matter can be prepared using organic group transformations known in the art of organic synthesis, as further described in the Examples below, and via methods analogous to those described in PCT International Publication No. 2020/214336, the disclosure of which is incorporated herein by reference in its entirety.

By way of example, SuTEx probes and ligands comprising a substituted 1,2,4-triazole group can be prepared as shown in Scheme 7, below. As shown in Scheme 7, an amide starting material (compound A in Scheme 7, where J represents the triazole substituent in the final SuTEx compound) can be coupled with DMF-DMA to produce an amidine intermediate (B). The amidine intermediate can undergo cyclization in acetic acid with hydrazine hydrate to form the corresponding 1,2,4-triazole^{[26]}, i.e., compound C in Scheme 7. The 1,2,4-triazole can then be reacted with a suitable sulfonyl chloride to provide the final SuTEx probe or ligand. J′ in Scheme 7 represents the AG of the SuTEx compound. Exemplary methods of preparing sulfonyl chlorides are described in the Examples below. See for example, Scheme 15 in Example 1, where suitable sulfonyl chlorides are prepared by reacting 4-(chlorosulfonyl)benzoyl chloride with an amine in the presence of diisopropylethylamine (DIPEA). Additional compounds for sulfur heterocycle exchange chemistry can be prepared by reacting the sulfonyl chlorides of with other N-heteroaryl compounds, e.g., imidazole, a substituted imidazole, pyrazole, a substituted pyrazole, tetrazole, or a substituted pyrazole.

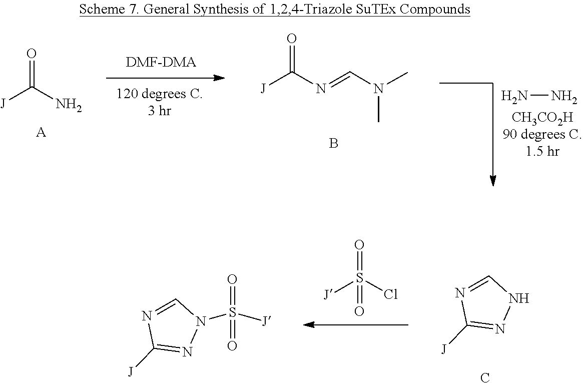

SuTEx probes comprising a 1,2,3-triazole group can be prepared as using a previously reported procedure^{[27]}, involving a copper catalyzed azide-alkyne cycloaddition using copper(I) thiophene-2-carboxylate (CuTC) in toluene. See Scheme 8, below. This initial cycloaddition provides a 1,4-regioisomer of the 1,2,3-triazole (compound D in Scheme 8), which can be converted to the 2,4-regioisomer^{[28]} (compound E) using dimethylaminopyridine (DMAP) in acetonitrile.

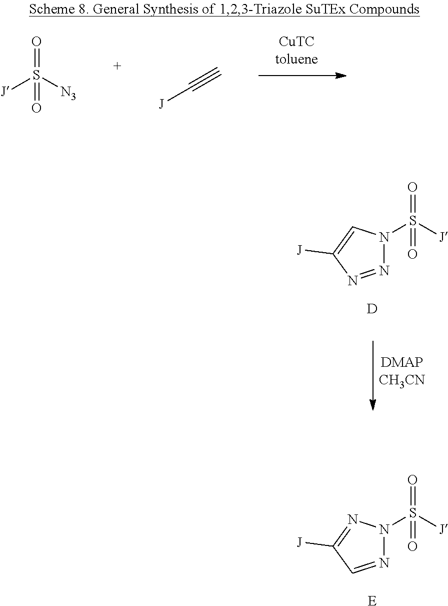

Alternatively, sulfonyl-triazole compounds can be prepared by synthetic routes involving a sulfide intermediate. Scheme 9, below, shows the synthesis of a sulfonyl-triazole compound by a route involving a benzyl sulfide intermediate.

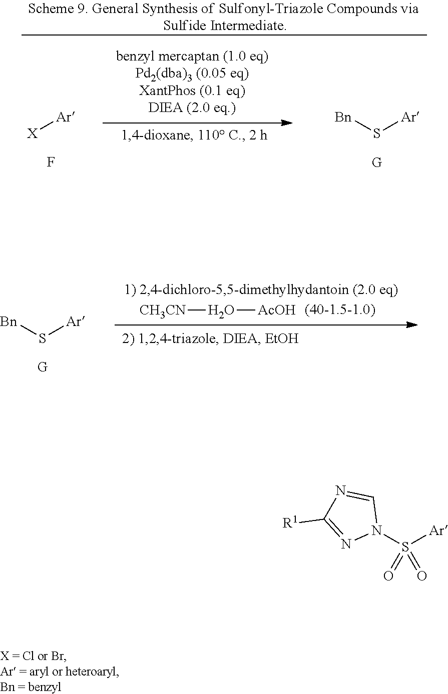

For example, as shown in Scheme 9, halo-substituted arene or heteroarene F can be reacted with benzyl mercaptan to provide benzyl sulfide intermediate G. Treatment of benzyl sulfide G with 1,2-dichloro-5,5-dimethylhydantoin in acetonitrile/water/acetic acid, followed by reaction with a 1,2,4-triazole provides the sulfonyl-triazole product. Other sulfonyl-heteroaryl compounds can be prepared by analogous routes using other nitrogen-containing heteroaryl compounds (e.g., imidazole) in place of the 1,2,4-triazole.

Routes to SuTEx compounds where the Ar₂ group of Formula (I) is attached to a Q group comprising a —C(═O)— moiety as Z₁, such as those shown in Scheme 4, above, can be prepared by a route using a sulfide intermediate where the Ar₂ group is substituted by a carboxylic acid moiety, which is then reacted with an amine that corresponds to the —Z₂—(NH)_{y}—R₁ group. See Scheme 10, below. Such sulfide intermediates can be prepared by reacting a suitable carboxylic acid-substituted haloarene with benzyl mercaptan in a step analogous to the first step in Scheme 9. Routes to SuTEx compounds wherein the A2 group of Formula (I) is attached to a Q group with a methylene moiety (i.e., where Z₁ is —CH—), such as those shown in Scheme 5 above, can be prepared by a route using a sulfide intermediate substituted by an aldehyde (—C(═O)—H, which can then undergo reductive amination with an amine that corresponds to the —Z₂—(NH)_{y}—R₁ group. See Scheme 11, below. Such sulfide intermediates can be prepared by reacting a suitable halo-substituted aromatic aldehyde with benzyl mercaptan in a step analogous to the first step in Scheme 9. Commercially available halo-substituted carboxylic acids and aldehydes for the preparation of heteroaryl sulfide intermediates comprising pyridine or pyrimidine groups are shown below in Scheme 12. Methods of preparing analogous halo-substituted triazine carboxylic acids and aldehydes are provided in Example 12, below. The sulfide products of the reactions shown in Schemes 10 and 11 can be transformed into sulfonyl-triazole compounds (or other sulfonyl-heteroaryl compounds) in a manner analogous to that shown in Scheme 9 for intermediate G.

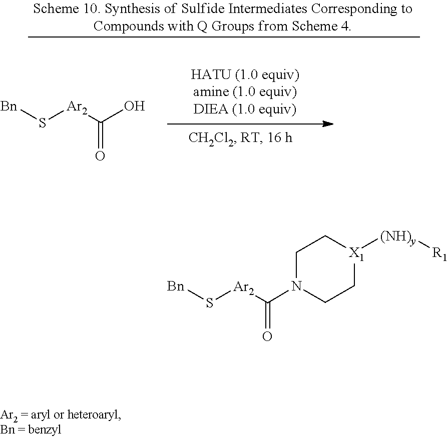

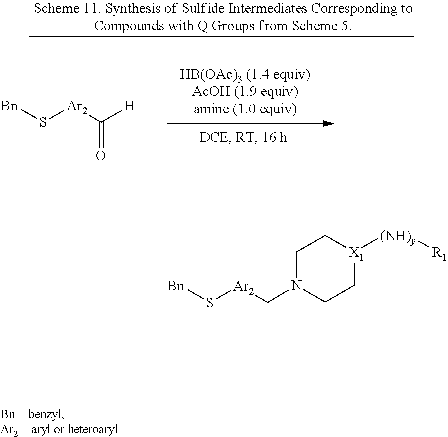

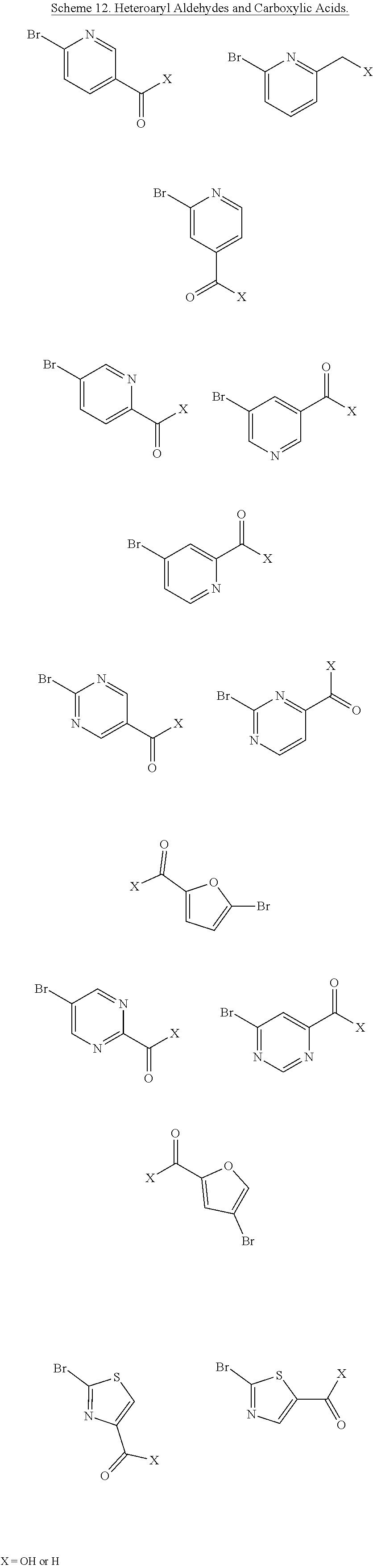

Amine reactants for use in the reactions with the benzyl sulfides shown in Schemes 10 and 11 can be prepared by, for example, Buchwald-Hartwig amination, Chan-Evans-Lam amination, or reductive amination, as described hereinbelow in Example 12. For instance, the amine reactant can be prepared from a nitrogen-protected starting material corresponding to the Z₂ group in Formula (I), such as one of the commercially available tert-butoxycarbonyl (Boc)-protected amines in Scheme 13, below, which can be reacted with an aryl halide or anilo compound corresponding to the R₁ group of Formula (I), such as one of the commercially available compounds shown in Scheme 14, below. Aryl halide or anilo compounds comprising a keto group can be transformed to the corresponding geminal difluorides for use as additional R₁ synthons via reaction with di ethylaminosulfur trifluoride (DAST), as described in Example 12. Then the nitrogen-protecting group can be removed and the resulting amine used as the amine (representing the Z₂—(NH)_{y}—R₁ group of Formula (I)) in the reaction shown in Scheme 10 or 11.

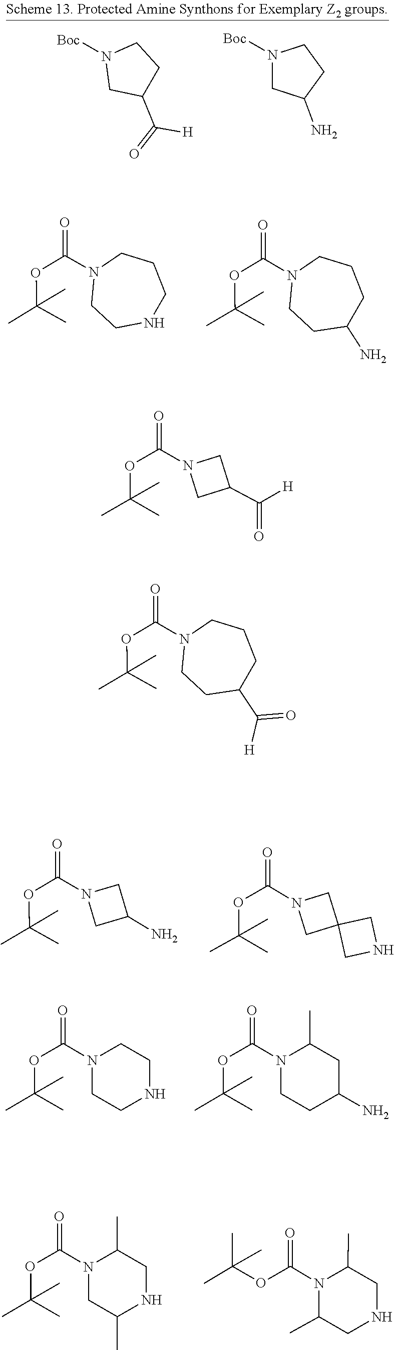

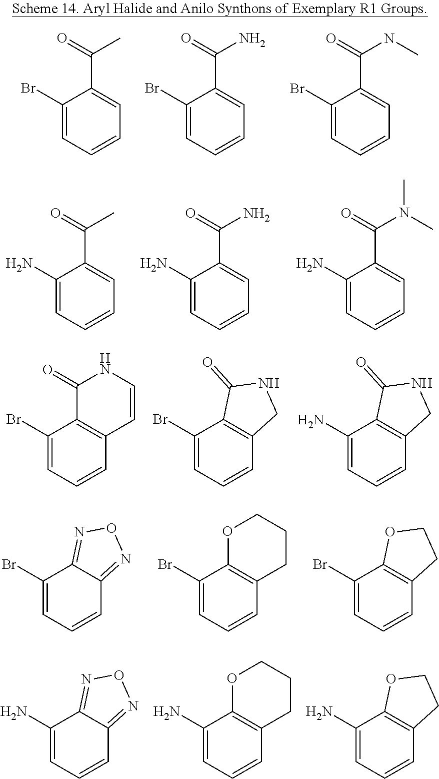

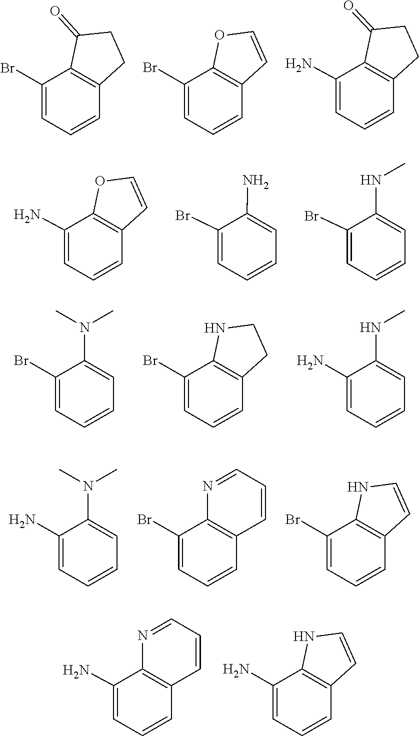

### V. Methods of Identifying Reactive Amino Acid Residues

Covalent probes can serve as valuable tools for the global investigation of protein function and ligand binding capacity. Despite efforts to expand coverage of residues available for chemical proteomics (e.g. cysteine and lysine), a large fraction of the proteome remains inaccessible with current activity-based probes. According to one aspect of the presently disclosed subject matter is described sulfur-heterocycle exchange chemistry (e.g., sulfur-triazole exchange (SuTEx) chemistry) as a tunable platform for developing covalent probes and ligands with broad applications for chemical proteomics. Sulfur-heterocycle probes and ligands can act as electrophiles for reactive nucleophilic amino acid side chains of proteins, where reaction of the nucleophilic group of the nucleophilic amino acid side chain with the sulfur-heterocycle probe results in formation of a covalent bond between the nucleophilic group and the sulfur atom of a sulfonyl group in the probe and the breaking of a bond between the sulfonyl group and the heterocycle.

As example of the tunability of this platform, in SuTEx probes, modifications to the triazole leaving group can furnish sulfonyl probes with ˜5-fold enhanced chemoselectivity for tyrosines over other nucleophilic amino acids to investigate, for the first time, more than 10,000 tyrosine sites in lysates and live cells. Tyrosines with enhanced nucleophilicity have been found to be enriched in enzymatic, protein-protein interaction, and nucleotide recognition domains. In addition, SuTEx can be used as a chemical phosphoproteomics strategy to monitor activation of phosphotyrosine sites. Accordingly, collectively, SuTEx and related sulfur-heterocycle exchange chemistry compounds provide a biocompatible chemistry for chemical biology investigations of the human proteome.

In some embodiments, the presently disclosed subject matter provides small molecule probes that interact with reactive nucleophilic residues on proteins or peptides, such as a reactive tyrosine residue of a tyrosine-containing protein and/or a reactive lysine residue of a lysine-containing protein, as well as methods of identifying a protein or peptide that contains such a reactive residue (e.g., a druggable tyrosine residue and/or a druggable lysine residue). In some instances, also described herein are methods of profiling a ligand that interacts with one or more tyrosine- and/or lysine-containing protein comprising one or more reactive tyrosines and/or lysines.

In some embodiments, the presently disclosed subject matter provides a method of identifying a reactive tyrosine of a protein, the method comprising: (a) providing a protein sample comprising isolated proteins, living cells, or a cell lysate; (b) contacting the protein sample with a probe compound as described hereinabove (e.g., a compound of Formula (V)) for a period of time sufficient for the probe compound to react with at least one reactive tyrosine in a protein in the protein sample, thereby forming at least one modified reactive tyrosine residue; and (c) analyzing proteins in the protein sample to identify at least one modified tyrosine residue, thereby identifying at least one reactive tyrosine of a protein. In some embodiments, the probe compound has a structure of the Formula:

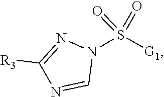

wherein: G₁ is a monovalent moiety comprising an alkyne moiety, a fluorophore moiety, a detectable labeling group, or a combination thereof, and R₃ is cycloalkyl, e.g., selected from cyclopropyl, cyclobutyl, cyclopentyl, and cyclohexyl. In some embodiments, the at least one modified reactive tyrosine residue comprises a modified tyrosine residue comprising a structure:

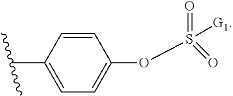

In some embodiments, G₁ comprises a fluorophore or detectable labeling moiety as described hereinbelow. In some embodiments, G₁ comprises an aryl group substituted by an alkyne-substituted alkyl group, an alkyne-substituted alkoxy group, or a group having the formula —C(═O)—NH-alkylene-C≡CH.

In some embodiments, the presently disclosed methods can alternatively or additionally provide for identifying reactive lysine residues in a protein. For example, during the contacting step (b) of the method described hereinabove, the probe compound can react with at least one reactive lysine in a protein in the protein sample, thereby forming at least one modified reactive lysine residue, and during the analyzing step (c), the method can further comprise analyzing the proteins in the protein sample to identify the at least one modified lysine residue, thereby identifying at least one reactive lysine of a protein.

Thus, in some embodiments, the presently disclosed subject matter provides a method of identifying a reactive lysine of a protein, the method comprising: (a) providing a protein sample comprising isolated proteins, living cells, or a cell lysate; (b) contacting the protein sample with a probe compound (e.g., of Formula (V)) for a period of time sufficient for the probe compound to react with at least one reactive lysine in a protein in the protein sample, thereby forming at least one modified reactive lysine residue; and (c) analyzing proteins in the protein sample to identify at least one modified lysine residue, thereby identifying at least one reactive lysine of a protein. In some embodiments, the at least one modified reactive lysine residue comprises a modified lysine residue comprising a structure:

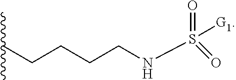

In some embodiments, the at least one modified reactive lysine residue is in a kinase.

In some embodiments, the presently disclosed subject matter provides a method of identifying a reactive tyrosine and/or a reactive lysine of a protein, the method comprising: (a) providing a protein sample comprising isolated proteins, living cells, or a cell lysate; (b) contacting the protein sample with a probe compound of Formula (V) for a period of time sufficient for the probe compound to react with at least one reactive tyrosine and/or at least one reactive lysine in a protein in the protein sample, thereby forming at least one modified reactive tyrosine residue and/or at least one modified reactive lysine residue, wherein the probe compound of Formula (V) has the structure:

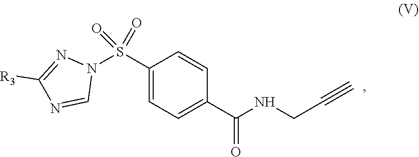

wherein R₃ is selected from cyclopropyl, cyclobutyl, cyclopentyl, and cyclohexyl; and (c) analyzing proteins in the protein sample to identify at least one modified tyrosine residue and/or at least one modified lysine residue, thereby identifying at least one reactive tyrosine and/or at least one reactive lysine of a protein; wherein the at least one modified reactive tyrosine residue comprises a modified tyrosine residue comprising a structure of Formula (VI):

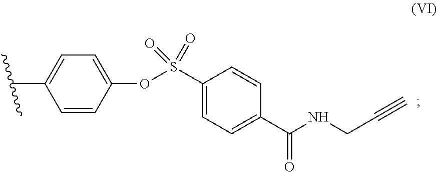 [SMILES: *c1ccc(OS(=O)(=O)c2ccc(C(=O)NCC#C)cc2)cc1]

and the at least one modified reactive lysine residue comprises a modified lysine residue comprising a structure of Formula (VII):

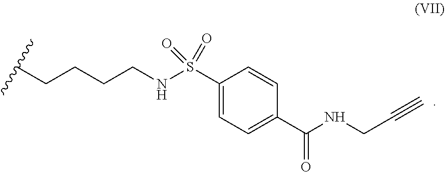 [SMILES: *CCCCNS(=O)(=O)c1ccc(C(=O)NCC#C)cc1]

In some embodiments, the probe compound of Formula (V) is selected from the group comprising: 4-((3-cyclopropyl-1H-1,2,4-triazol-1-yl)sulfonyl)-N-(prop-2-yn-1-yl)benzamide (RJG-2043), 4-((3-cyclobutyl-1H-1,2,4-triazol-1-yl)sulfonyl)-N-(prop-2-yn-1-yl)benzamide (RJS-1027), 4-((3-cyclopentyl-1H-1,2,4-triazol-1-yl)sulfonyl)-N-(prop-2-yn-1-yl)benz-amide (RJG-2044), and 4-((3-cyclohexyl-1H-1,2,4-triazol-1-yl)sulfonyl)-N-(prop-2-yn-1-yl)benzamide (RJG-2045).

In some embodiments, the analyzing of step (c) further comprises tagging the at least one modified reactive tyrosine residue and/or at least one reactive lysine residue with a compound comprising a detectable labeling group, thereby forming at least one tagged reactive tyrosine residue comprising said detectable labeling group and/or at least one tagged reactive lysine residue comprising said detectable labeling group. In some embodiments, the detectable labeling group comprises biotin or a biotin derivative. In some embodiments, the biotin derivative is desthiobiotin.

In some embodiments, the tagging comprises reacting an alkyne group of at least one tagged reactive tyrosine residue and/or at least one tagged reactive lysine residue with a compound comprising both an azide moiety (or other alkyne-reactive group) and a detectable labeling group (e.g., biotin or a biotin derivative. In some embodiments, the compound comprising the azide moiety and the detectable labeling group further comprises an alkylene linker, which in some embodiments, can comprise a polyether group, such as an oligomer of methylene glycol, ethylene glycol or propylene glycol (e.g., a group having the formula —(O—C₂H₄—)ₓ—). In some embodiments, the tagging comprises performing a copper-catalyzed azide-alkyne cycloaddition (CuAAC) coupling reaction.

In some embodiments, the analyzing further comprises digesting the protein sample to provide a digested protein sample comprising a protein fragment comprising the at least one tagged reactive tyrosine moiety comprising the detectable group and/or the at least one tagged reactive lysine residue comprising the detectable group. In some embodiments, the digesting is performed with a peptidase. In some embodiments, the digesting is performed with trypsin.

In some embodiments, the analyzing further comprises enriching the digested protein sample for the detectable labeling group. For example, in some embodiments, the enriching comprises contacting the digested protein sample with a solid support comprising a binding partner of the detectable labeling group. In some embodiments, when the detectable labeling group comprises biotin or a derivative thereof, the solid support comprises streptavidin. In some embodiments, the analyzing further comprises analyzing the digested protein sample (e.g., the enriched digested protein sample) via liquid chromatography-mass spectrometry or via a gel-based assay.

In some embodiments, providing the protein sample further comprises separating the protein sample into a first protein sample and a second protein sample. Then, in the contacting step, the first protein sample can be contacted with a first probe compound (e.g., a probe compound of Formula (V)) at a first probe concentration for a first period of time and the second protein sample can be contacted with a second probe compound (e.g., a second probe compound of Formula (V) having a different structure than that of the first probe compound) at the same probe concentration (i.e., at the first probe concentration) for the same time period (i.e., for the first period of time. Alternatively, the second protein sample can be contacted with the same probe compound as the first protein sample, but at a different probe concentration (i.e., a second probe concentration) or for a different period of time. In some embodiments, analyzing proteins comprises analyzing the first and second protein samples to determine the presence and/or identity of a modified reactive tyrosine and/or lysine residue in the first sample and the presence and/or identity of a modified reactive tyrosine and/or lysine residue in the second sample. In some embodiments, the identities and/or amounts of identified modified reactive tyrosine and/or lysine residues from the first and second protein samples are compared.

In some embodiments, the protein sample comprises living cells. In some embodiments, providing the protein sample further comprises separating the protein sample into a first protein sample and a second protein sample and culturing the first protein sample in a first cell culture medium comprising heavy isotopes prior to the contacting of step (b) and culturing the second protein sample in a second cell culture medium, wherein the second culture medium comprises a naturally occurring isotope distribution prior to the contacting of step (b). In some embodiments, the first cell culture medium comprises ¹³C- and/or ¹⁵N-labeled amino acids. In some embodiments, the first cell culture medium comprises ¹³C-¹⁵N-labeled lysine and arginine.

In some embodiments, e.g., if the protein sample does not comprise living cells, the probe compound can comprise a detectable labeling group comprising a heavy isotope (e.g., a ¹³C label) or the method can comprise tagging the at least one modified tyrosine residue and/or at least one modified lysine residue with a detectable labeling group comprising a heavy isotope.

In some embodiments, the protein sample is separated into a first and a second protein sample and one of the first and the second protein sample is cultured in the presences of a tyrosine phosphatase inhibitor (e.g., pervanadate). Thus, in some embodiments, the presently disclosed methods can be used in phosphoproteomics.

### VII. Modified Proteins

In some embodiments, the presently disclosed subject matter provides a modified tyrosine- and/or lysine-containing protein. The modified protein can be a protein comprising the adduct formed between a tyrosine phenol group or a lysine primary amino group and a probe or ligand of the presently disclosed subject matter. The modified protein can have a different biological activity than the unmodified protein.

In some embodiments, the presently disclosed subject matter provides a modified tyrosine-containing protein comprising modified tyrosine residue comprising a structure:

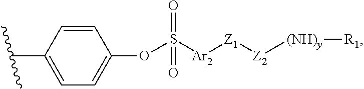

wherein y, Ar₂, Z₁, Z₂, and R₁ are as defined hereinabove for the compounds of Formula (I). In some embodiments, Ar₂ is phenyl. In some embodiments, Z₂ is piperazinyl or piperidinyl. In some embodiments, R₁ is selected from aryl, heteroaryl, substituted aryl, or substituted heteroaryl. In some embodiments, R₁ is selected from phenyl, substituted phenyl (e.g., 2-methoxyphenyl), pyridyl, or substituted pyridyl. In some embodiments, a modified lysine-containing protein comprising a modified lysine reside (i.e., wherein the lysine side chain amino group has formed a covalent bond with a sulfonyl group that is further attached to an —Ar₂—Z₁-Z₂—(NH)_{y}—R₁) can be provided.

In some embodiments, the presently disclosed subject matter provides a modified tyrosine-containing protein comprising a modified tyrosine residue wherein the modified tyrosine residue is formed by the reaction of a tyrosine residue with a non-naturally occurring compound having a structure of Formula (I), (II), (III), (IIIa), (Tub), (IV), (IVa), (IVb), or (I′).

The modified tyrosine-containing protein can be a protein that comprises a tyrosine residue as denoted in Table 2. In some embodiments, the modified tyrosine-containing protein is modified at a tyrosine residue in prostaglandin reductase 2 (PTGR2). In some embodiments, the modified tyrosine residue at position 100 of PTGR2 is a tyrosine modified by AMC-703, RJG-1189, or RJG-2036, or a pharmaceutically acceptable salt thereof. In some embodiments, the modified tyrosine residue at position 265 of PTGR2 is a tyrosine modified by AMC-703, RJG-1189, or RJG-2036, or a pharmaceutically acceptable salt thereof.

In some embodiments, the presently disclosed subject matter provides a modified lysine-containing protein comprising a modified lysine residue wherein the modified lysine residue is formed by the reaction of a lysine residue with a non-naturally occurring compound having a structure of Formula (I), (II), (III), (IIIa), (IIIb), (IV), (IVa), (IVb), or (I′).

### VIII. Therapeutic Uses and Pharmaceutical Compositions

Small molecules, such as the presently disclosed ligands and probes, present an alternative method to selectively modulate proteins and to serve as leads for the development of novel therapeutics.

Dysregulated expression of a tyrosine-containing protein, in many cases, is associated with or modulates a disease, such as an inflammatory related disease, a neurodegenerative disease, or cancer. As such, identification of a potential agonist/antagonist to a tyrosine-containing protein aids in improving the disease condition in a patient.

Thus, in some embodiments, disclosed herein are tyrosine-containing proteins that comprise one or more ligandable tyrosines. In some instances, the tyrosine-containing protein is a soluble protein or a membrane protein. In some instances, the tyrosine-containing protein is involved in one or more of a biological process such as protein transport, lipid metabolism, apoptosis, transcription, electron transport, mRNA processing, or host-virus interaction. In some instances, the tyrosine-containing protein is associated with one or more of diseases such as cancer or one or more disorders or conditions such as immune, metabolic, developmental, reproductive, neurological, psychiatric, renal, cardiovascular, or hematological disorders or conditions.

In some embodiments, disclosed herein are lysine-containing proteins that comprise one or more ligandable lysines. In some instances, the lysine-containing protein is a soluble protein. In other instances, the lysine-containing protein is a membrane protein. In some cases, the lysine-containing protein is involved in one or more of a biological process such as protein transport, lipid metabolism, apoptosis, transcription, electron transport, mRNA processing, or host-virus interaction. In additional cases, the lysine-containing protein is associated with one or more of diseases such as cancer or one or more disorders or conditions such as immune, metabolic, developmental, reproductive, neurological, psychiatric, renal, cardiovascular, or hematological disorders or conditions.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the presently disclosed subject matter provides pharmaceutical compositions comprising one or more of the presently disclosed ligands or probes. The pharmaceutical compositions comprise at least one disclosed compound, e.g. selected from compounds of Formula (I), (II), III), (IIIa), (Tub), (IV), (IVa), (IVb), or (I′) described herein, or a pharmaceutically acceptable salt thereof, in combination with a pharmaceutically acceptable carrier, vehicle, or diluent, such as an aqueous buffer at a physiologically acceptable pH (e.g., pH 7 to 8.5), a non-aqueous liquid, a polymer-based nanoparticle vehicle, a liposome, and the like. The pharmaceutical compositions can be delivered in any suitable dosage form, such as a liquid, gel, solid, cream, or paste dosage form. In one embodiment, the compositions can be adapted to give sustained release of the active compound.

In some embodiments, the pharmaceutical compositions include, but are not limited to, those forms suitable for oral, rectal, nasal, topical, (including buccal and sublingual), transdermal, vaginal, parenteral (including intramuscular, subcutaneous, and intravenous), spinal (epidural, intrathecal), central (intracerebroventricular) administration, in a form suitable for administration by inhalation or insufflation. The compositions can, where appropriate, be provided in discrete dosage units. The pharmaceutical compositions of the invention can be prepared by any of the methods well known in the pharmaceutical arts. Some preferred modes of administration include intravenous (i.v.), intraperitoneal (i.p.), topical, subcutaneous, and oral.

Pharmaceutical formulations suitable for oral administration include capsules, cachets, or tablets, each containing a predetermined amount of one or more of the ligands, as a powder or granules. In another embodiment, the oral composition is a solution, a suspension, or an emulsion. Alternatively, the ligands can be provided as a bolus, electuary, or paste. Tablets and capsules for oral administration can contain conventional excipients such as binding agents, fillers, lubricants, disintegrants, colorants, flavoring agents, preservatives, or wetting agents. The tablets can be coated according to methods well known in the art, if desired. Oral liquid preparations include, for example, aqueous or oily suspensions, solutions, emulsions, syrups, or elixirs. Alternatively, the compositions can be provided as a dry product for constitution with water or another suitable vehicle before use. Such liquid preparations can contain conventional additives such as suspending agents, emulsifying agents, non-aqueous vehicles (which may include edible oils), preservatives, and the like. The additives, excipients, and the like typically will be included in the compositions for oral administration within a range of concentrations suitable for their intended use or function in the composition, and which are well known in the pharmaceutical formulation art. The presently disclosed ligands will be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions for parenteral, spinal, or central administration (e.g. by bolus injection or continuous infusion) or injection into amniotic fluid can be provided in unit dose form in ampoules, pre-filled syringes, small volume infusion, or in multi-dose containers, and preferably include an added preservative. The compositions for parenteral administration can be suspensions, solutions, or emulsions, and can contain excipients such as suspending agents, stabilizing agents, and dispersing agents. Alternatively, the ligands can be provided in powder form, obtained by aseptic isolation of sterile solid or by lyophilization from solution, for constitution with a suitable vehicle, e.g. sterile, pyrogen-free water, before use. The additives, excipients, and the like typically will be included in the compositions for parenteral administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 100 millimolar, preferably at least about 1 nanomolar to about 10 millimolar.

Pharmaceutical compositions for topical administration of the ligands to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like. The additives, excipients, and the like typically will be included in the compositions for topical administration to the epidermis within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions suitable for topical administration in the mouth (e.g., buccal or sublingual administration) include lozenges comprising the ligand in a flavored base, such as sucrose, acacia, or tragacanth; pastilles comprising the ligand in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired. The additives, excipients, and the like typically will be included in the compositions of topical oral administration within a range of concentrations suitable for their intended use or function in the composition, and which are well known in the pharmaceutical formulation art. The ligands of the presently disclosed subject matter invention can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

A pharmaceutical composition suitable for rectal administration comprises a ligand of the presently disclosed subject matter in combination with a solid or semisolid (e.g., cream or paste) carrier or vehicle. For example, such rectal compositions can be provided as unit dose suppositories. Suitable carriers or vehicles include cocoa butter and other materials commonly used in the art. The additives, excipients, and the like typically will be included in the compositions of rectal administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

According to one embodiment, pharmaceutical compositions of the present invention suitable for vaginal administration are provided as pessaries, tampons, creams, gels, pastes, foams, or sprays containing a ligand of the presently disclosed subject matter in combination with a carriers as are known in the art. Alternatively, compositions suitable for vaginal administration can be delivered in a liquid or solid dosage form. The additives, excipients, and the like typically will be included in the compositions of vaginal administration within a range of concentrations suitable for their intended use or function in the composition, and which are well known in the pharmaceutical formulation art. The ligands of the presently disclosed subject matter will be included in the compositions within a therapeutically useful and effective concentration range, as determined by routine methods that are well known in the medical and pharmaceutical arts. For example, a typical composition can include one or more of the presently disclosed ligands at a concentration in the range of at least about 0.01 nanomolar to about 1 molar, preferably at least about 1 nanomolar to about 100 millimolar.

Pharmaceutical compositions suitable for intra-nasal administration are also encompassed by the present invention. Such intra-nasal compositions comprise a ligand of the presently disclosed subject matter in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising the ligand. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the ligand. Alternatively, pharmaceutical compositions for administration by inhalation or insufflation can be provided in the form of a dry powder composition, for example, a powder mix of the ligand and a suitable powder base such as lactose or starch. Such powder composition can be provided in unit dosage form, for example, in capsules, cartridges, gelatin packs, or blister packs, from which the powder can be administered with the aid of an inhalator or insufflator. The additives, excipients, and the like typically will be included in the compositions of intra-nasal administration within a range of concentrations suitable for their intended use or function in the composition, and which are well known in the pharmaceutical formulation art. The ligand of the presently disclosed subject matter will be included in the compositions within a therapeutically useful and effective concentration range, as determined by routine methods that are well known in the medical and pharmaceutical arts. For example, a typical composition can include one or more ligand at a concentration in the range of at least about 0.01 nanomolar to about 1 molar, preferably at least about 1 nanomolar to about 100 millimolar.

Optionally, the pharmaceutical compositions of the presently disclosed subject matter can include one or more other therapeutic agent, e.g., as a combination therapy. The additional therapeutic agent will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration can be lower than a typical monotherapy concentration if there is a synergy when combined with a ligand of the presently disclosed subject matter.

In some embodiments, the presently disclosed subject matter provides a method of inhibiting prostaglandin reductase 2 (PTGR2), wherein the method comprises contacting a sample comprising PTGR2 with an effective amount of a ligand compound as described hereinabove, i.e., a compound of Formula (I), (II), (III), (IIIa), (Tub), (IV), (IVa), (IVb), or (I′) or a pharmaceutically acceptable salt thereof and/or a pharmaceutical composition thereof. Thus, in some embodiments, the presently disclosed compounds can act as PTGR2 inhibitors. The amino acid sequence of human PTGR2 (Uniprot ID Q8N8N7) is: MIVQRVVLNS RPGKNGNPVA ENFRMEEVYL PDNINEGQVQ VRTLYLSVDP YMRCRMNEDT GTDYITPWQL SQVVDGGGIG IIEESKHTNL TKGDFVTSFY WPWQTKVILD GNSLEKVDPQ LVDGHLSYFL GAIGMPGLTS LIGIQEKGHI TAGSNKTMVV SGAAGACGSV AGQIGHFLGC SRVVGICGTH EKCILLTSEL GFDAAINYKK DNVAEQLRES CPAGVDVYFD NVGGNISDTV ISQMNENSHI ILCGQISQYN KDVPYPPPLS PAIEAIQKER NITRERFLVL NYKDKFEPGI LQLSQWFKEG KLKIKETVIN GLENMGAAFQ SMMTGGNIGK QIVCISEEIS L (SEQ ID NO: 3).

PTGR2 has been found to be overexpressed in human pancreatic adenocarcinoma. PTGR2 also has been implicated in peroxisome proliferator-activated receptor (PPAR)-gamma (γ) signaling, which is involved in the development of metabolic syndrome and inflammatory diseases. Accordingly, modulation of PTGR2 presents an attractive therapeutic approach for treating cancer and/or PPARγ-related disorders (e.g., obesity, diabetes, and atherosclerosis). In some embodiments, the compound used in inhibiting PTGR2 is selected from AMC-0703, RJG-1189, and RJG-2036, or a pharmaceutically acceptable salt and/or a pharmaceutical composition thereof.

The sample comprising PTGR2 can be, for example, a biological sample, such as, but not limited to, a biological fluid, a cell, a cell culture, a cell extract, a tissue, a tissue extract, an organ or an organism (e.g., a living organism, such as a human or other mammal). In some embodiments, inhibiting the PTGR2 can treat and/or prevent a disease or disorder associated with PTGR2 (e.g., increased PTGR2), such as, but not limited to, cancer, obesity, diabetes, and atherosclerosis. Thus, in some embodiments, the presently disclosed subject matter presents a method of treating a disease or disorder in a subject in need thereof, wherein the method comprises administering to the subject a compound of one of Formulas (I), (II), (III), (IIIa), (Tub), (IV), (IVa), (IVb), or (I′), or a pharmaceutically acceptable salt and/or pharmaceutical composition thereof. In some embodiments, the compound is selected from AMC-0703, RJG-1189, and RJG-2036.

In some embodiments, the presently disclosed subject matter provides a pharmaceutical composition for use in inhibiting PTGR2 in a subject, wherein the pharmaceutical composition comprises a compound of one of Formulas (I), (II), (III), (IIIa), (IIIb), (IV), (IVa), (IVb), or (I′) or a pharmaceutically acceptable salt and/or pharmaceutical composition thereof. In some embodiments, the presently disclosed subject matter provides a pharmaceutical composition for use in treating a disease or disorder treatable by inhibiting PTGR2 (e.g., cancer, obesity, diabetes, or atherosclerosis) in a subject, wherein the pharmaceutical composition comprises a compound of one of Formulas (I), (II), (III), (IIIa), (IIIb), (IV), (IVa), (IVb), or (I′) or a pharmaceutically acceptable salt and/or pharmaceutical composition thereof.

### IX. Cells, Analytical Techniques and Instrumentation

In some embodiments, one or more of the methods disclosed herein comprise a sample (e.g., a cell sample, or a cell lysate sample). In some embodiments, the sample for use with the methods described herein is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the sample for use with the methods described herein is obtained from a mammalian cell. In some instances, the mammalian cell is an epithelial cell, connective tissue cell, hormone secreting cell, a nerve cell, a skeletal muscle cell, a blood cell, or an immune system cell. Exemplary mammalian cell lines include, but are not limited to, 293A cells, 293FT cells, 293F cells, 293H cells, HEK 293 cells, CHO DG44 cells, CHO—S cells, CHO-K1 cells, and PC12 cells.

In some embodiments, the sample for use with the methods described herein is obtained from cells of a tumor cell line. In some instances, the sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, and telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CIVIL), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the sample for use with the methods described herein is obtained from a tumor cell line. Exemplary tumor cell lines include, but are not limited to, 600MPE, AU565, BT-20, BT-474, BT-483, BT-549, Evsa-T, Hs578T, MCF-7, MBA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the sample for use in the methods is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the sample is a tissue sample, such as a sample obtained from a biopsy or a tumor tissue sample. In some embodiments, the sample is a blood serum sample. In some embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

### X. Sample Preparation and Analysis

In some embodiments, the sample (e.g., cell sample, cell lysate sample, or comprising isolated proteins) is a sample solution. In some instances, the sample solution comprises a solution such as a buffer (e.g. phosphate buffered saline) or a media. In some embodiments, the media is an isotopically labeled media. In some instances, the sample solution is a cell solution.

In some embodiments, the sample (e.g., cell sample, cell lysate sample, or comprising isolated proteins) is incubated with one or more compound probes for analysis of protein-probe interactions. In some instances, the sample (e.g., cell sample, cell lysate sample, or comprising isolated proteins) is further incubated in the presence of an additional compound probe prior to addition of the one or more probes. In other instances, the sample (e.g., cell sample, cell lysate sample, or comprising isolated proteins) is further incubated with a non-probe small molecule ligand, in which the non-probe small molecule ligand does not contain a photoreactive moiety and/or an alkyne group. In such instances, the sample is incubated with a probe and non-probe small molecule ligand for competitive protein profiling analysis.

In some cases, the sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a sample (e.g. cell sample, cell lysate sample, or comprising isolated proteins) with a labeling group (e.g., an isotopically labeled labeling group) to tag one or more proteins of interest for further analysis. In such cases, the detectable labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical approach. In some instances, the protein from the probe-protein complexes is fragmented by a chemical approach. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage KIF endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electro spray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method.

In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ¹⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by a method as previously described. See PCT International Publication No. 2020/214336, the disclosure of which is incorporated herein by reference in its entirety.

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, New York, United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, New York, United States of America; and Ausubel et al. (1995) Current Protocols in Molecular Biology, Greene Publishing.

### XI. Kits/Articles of Manufacture

Disclosed herein, in certain embodiments, are kits and articles of manufacture for use with one or more methods described herein. In some embodiments, described herein is a kit for generating a protein comprising a detectable group and/or a fragment of a ligand compound described herein. In some embodiments, such kit includes a probe or ligand as described herein, small molecule fragments or libraries, and/or controls, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use. For example, the container(s) include probes, ligands, control compounds, and one or more reagents for use in a method disclosed herein.

The presently disclosed kits and articles of manufacture optionally include an identifying description or label or instructions relating to its use in the methods described herein. For example, a kit typically includes labels listing contents and/or instructions for use, and package inserts with instructions for use. A set of instructions will also typically be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### SuTEx Ligand and Probe Synthesis

All chemicals used were reagent grade and used as supplied, except where noted. N,N-Dimethylformamide (DMF), dichloromethane (DCM), toluene and tetrahydrofuran (THF) were used without any further purification steps. Analytical thin layer chromatography (TLC) was performed on Merck silica gel 60 F254 plates (0.25 mm). Flash column chromatography was carried out using forced flow of the indicated solvent on Silica Gel 60 (230-400 mesh) purchased from Fisher Scientific (Hampton, New Hampshire, United States of America). Compounds were visualized by UV-irradiation and iodine chamber. ¹H and ¹³C NMR spectra were recorded on a Varian Inova 500 (500 MHz) or 600 (600 MHz) spectrometer (Varian, Inc., Palo Alto, California, United States of America), or a Bruker Avance III 800 (800 MHz) spectrometer (Bruker, Billerica, Massachusetts, United States of America) in CDCl₃, Acetone-d₆, or DMSO-d₆ with chemical shifts referenced to internal standards (CDCl₃: 7.26 ppm ¹H, 77.16 ppm ¹³C; (CD₃)₂CO: 2.05 ppm ¹H, 29.84 and 206.26 ppm ¹³C; (CD₃)₂CO: 2.50 ppm ¹H, 40.00 ppm ¹³C) unless stated otherwise. Splitting patterns are indicated as s, singlet; d, doublet; t, triplet; q, quartet; m, multiplet; br, broad singlet for ¹H-NMR data. NMR chemical shifts (δ) are reported in ppm and coupling constants (J) are reported in Hz. High resolution mass spectral (FIRMS) data were obtained by an Agilent 6545B LC/Q-TOF (Agilent Technologies, Santa Clara, California, United States of America). High-performance liquid chromatography (HPLC) data was obtained by a Shimadzu 1100 Series spectrometer (Shimadzu, Kyoto, Japan) with UV detection at 254 nm using a reverse-phase column with a 10-minute acidified water/acetonitrile gradient as previously described^{[8a]}.

The following chemicals were purchased commercially: propargylamine and N,N-Dimethylformamide dimethyl acetal (both from Fisher Scientific, Hampton, New Hampshire, United States of America); Water, Acetonitrile, Dichloromethane, Acetone, Ethyl acetate, n-Heptane, Hexanes, Formic acid (sold under the tradename OPTIMA™) Acetic acid (sold under the tradename OPTIMA™), Sodium sulfate (all from Fisher Chemical, Thermo Fisher Scientific, Waltham, Massachusetts, United States of America); 4-(chlorosulfonyl)benzoyl chloride (Combi-Blocks, San Diego, California, United States of America); N,N-Diisopropylethylamine, 4-Phenylpiperidine, 1,2,4-1H-Triazole (all from Acros Organics, Geel, Belgium), and cyclopropylamine (Oakwood Chemicals, Estill, South Carolina, United States of America).

SuTEx ligand synthesis

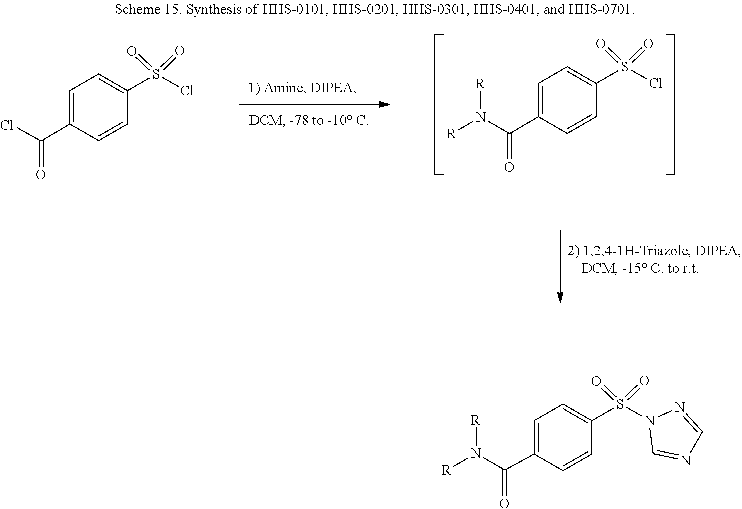

### General Synthesis Protocol:

Exemplary SuTEx ligands were prepared as shown in Scheme 15, above. To a solution of 4-(chlorosulfonyl) benzoyl chloride (0.42 mmol, 1.0 eq.) in anhydrous DCM (0.2 M) was added the corresponding amine (0.42 mmol, 1.0 eq.) and N,N-diisopropylethylamine (DIPEA) (0.92 mmol, 1.1 eq.) at −78° C. The reaction mixture was slowly warmed up to 0° C., and then to the reaction mixture was added the 1,2,4-triazole (1.26 mmol, 3.0 eq.) and N,N-diisopropylethylamine (DIPEA) (0.40 mmol, 1.1 eq.) at −15° C. Then the reaction mixture was warmed up to room temperature and stirred overnight. The crude product was directly loaded and purified using silica gel flash column chromatography (acetone/DCM=2:100 to 10:100) to afford HHS-0201, -0301, and -0401. The crude products for HHS-0101 and HHS-0701 were washed with brine and extracted with DCM, dried with sodium sulfate, collected via vacuum filtration, and concentrated. They were then redissolved in minimal DCM/Acetone (99/1), and loaded onto a 10 g silica chromatography column sold under the tradename BIOTAGE® Sfar HCD (Biotage AG, Uppsala, Sweden) eluted using a DCM/Acetone gradient (starting at 0% acetone and progressing to 2%, then 5%, then 10%) with a chromatography system sold under the tradename BIOTAGE® ISOLERA™ One (Biotage AG, Uppsala, Sweden). Fractions were analyzed by TLC and product fractions were combined and concentrated to yield HHS-0701. Combined fractions for HHS-0101 were recrystallized in hot ethyl acetate and n-heptane, collected using vacuum filtration, and concentrated to yield HHS-0101.

### 4-((1H-1,2,4-triazol-1-yl)sulfonyl)-N-cyclopropylbenzamide (HHS-0101)

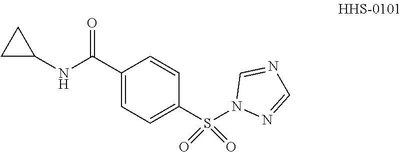 [SMILES: O=C(NC1CC1)c1ccc(S(=O)(=O)n2cncn2)cc1]

Yield: 22.9% as a white, fluffy solid, ¹H NMR (500 MHz, DMSO-d₆) δ 9.46 (s, 1H), 8.75-8.74 (d, J=5.0 Hz, 1H), 8.38 (s, 1H), 8.19-8.17 (m, 2H), 8.07-8.05 (m, 2H), 2.87-2.86 (m, 1H), 0.73-0.71 (m, 2H), 0.58-0.57 (m, 2H). ¹³C NMR (201 MHz, DMSO-d₆) δ 166.25, 155.29, 147.17, 141.45, 137.46, 129.35, 128.86, 128.46, 127.2, 23.67, 6.19, 5.56. ESI-gToF (HRMS) m/z [M+H]⁺ (formula: C₁₂H₁₃N₄O₃S⁺) calculated: 293.0703, found: 293.0703.

### 4-((1H-1,2,4-triazol-1-yl)sulfonyl)-N-(cyclopropylmethyl)benzamide (HHS-0201)

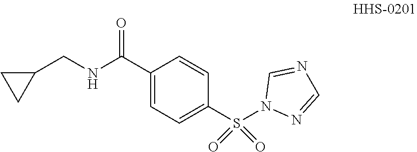 [SMILES: O=C(NCC1CC1)c1ccc(S(=O)(=O)n2cncn2)cc1]

Yield: 44% as a white, fluffy solid, ¹H NMR (600 MHz, Acetone-d₆) δ 9.12 (s, 1H), 8.21-8.20 (m, 1H), 8.19-8.18 (m, 2H), 8.15-8.12 (m, 3H), 3.27-3.25 (m, 2H), 1.10-1.05 (m, 1H), 0.47-0.44 (m, 2H), 0.26-0.24 (m, 2H). ¹³C NMR (150 MHz, Acetone-d₆) δ 165.50, 155.53, 146.73, 142.66, 138.73, 129.56, 129.56, 45.31, 45.17, 11.55, 3.83. ESI-qToF (HRMS) m/z [M+H]⁺ (formula: C₁₃H₁₅N₄O₃S⁺) calculated: 307.0859, found: 307.0859.

### 4-((1H-1,2,4-triazol-1-yl)sulfonyl)-N-cyclohexylbenzamide (HHS-0301)

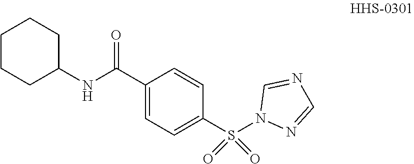 [SMILES: O=C(NC1CCCCC1)c1ccc(S(=O)(=O)n2cncn2)cc1]

Yield: 58% as a white, fluffy solid, ¹H NMR (600 MHz, Acetone-d₆) δ 9.12 (s, 1H), 8.20-8.17 (m, 3H), 8.14-8.10 (m, 2H), 7.76 (s, 1H), 3.89 (m, 1H), 2.00-1.92 (m, 2H), 1.80-1.74 (m, 2H), 1.69-1.61 (m, 1H), 1.44-1.29 (m, 4H), 1.25-1.13 (m, 1H). ¹³C NMR (150 MHz, Acetone-d₆) δ 164.76, 155.52, 146.71, 142.98, 138.62, 129.58, 129.47, 50.05, 33.42, 26.33, 25.87. ESI-gToF (HRMS) m/z [M+H]⁺ (formula: C₁₅H₁₉N₄O₃S⁺) calculated: 335.1172, found: 335.1174.

### 4-((1H-1,2,4-triazol-1-yl)sulfonyl)-N-benzylbenzamide (HHS-0401)

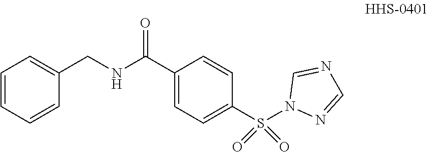 [SMILES: O=C(NCc1ccccc1)c1ccc(S(=O)(=O)n2cncn2)cc1]

Yield: 71% as a white, fluffy solid, ¹H NMR (600 MHz, Acetone-d₆) δ 9.13 (s, 1H), 8.51 (br s, 1H), 8.23-8.21 (m, 2H), 8.21-8.20 (m, 2H), 8.19 (s, 1H), 7.39-7.36 (m, 2H), 7.34-7.29 (m, 2H), 7.28-7.22 (m, 1H), 4.62 (d, J=6.0 Hz, 2H). ¹³C NMR (150 MHz, Acetone-d₆) δ 165.56, 155.54, 146.74, 142.31, 139.99, 138.93, 129.66, 129.61, 129.27, 128.51, 127.93, 44.26. ESI-gToF (HRMS) m/z [M+H]⁺ (formula: C₁₆H₁₅N₄O₃S⁺) calculated: 343.0859, found: 343.0859.

### (4-((1H-1,2,4-triazol-1-yl)sulfonyl)phenyl)(4-phenylpiperidin-1-yl)methanone (HHS-0701)

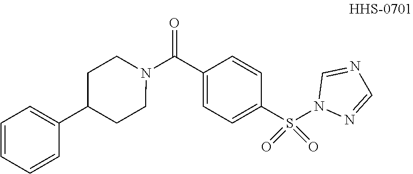 [SMILES: O=C(c1ccc(S(=O)(=O)n2cncn2)cc1)N1CCC(c2ccccc2)CC1]

Yield: 14.4% as a light yellow, fluffy solid, ¹H NMR (500 MHz, DMSO-d₆) δ 9.46 (s, 1H), 8.40 (s, 1H), 8.18-8.15 (m, 2H), 7.79-7.77 (m, 2H), 7.32-7.27 (m, 4H), 7.22-7.18 (m, 1H), 4.63-4.61 (d, J=10.0 Hz, 1H), 3.47-3.45 (d, J=10.0 Hz, 1H), 3.19-3.17 (t, J 5.0 Hz, 1H), 2.85-2.79 (m, 2H), 1.89-1.86 (d, J=15.0 Hz, 1H), 1.67-1.61 (m, 3H). ¹³C NMR (201 MHz, DMSO-d₆) δ 167.14, 155.29, 147.15, 145.91, 144.04, 136.00, 129.15, 128.86, 128.77, 127.24, 126.72, 47.97, 42.44, 42.10, 33.45, 32.97. ESI-gToF (HRMS) m/z [M+H]⁺ (formula: C₂₀H₂₁N₄O₃S⁺) calculated: 397.1329, found: 397.1330.

### Probe Synthesis:

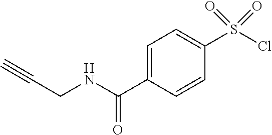 [SMILES: C#CCNC(=O)c1ccc(S(=O)(=O)Cl)cc1]

### 4-(Prop-2-yn-1-ylcarbamoyl)benzenesulfonyl chloride, S1 (1)

To a solution of 4-(chlorosulfonyl)benzoic acid (1.8 g, 8.2 mmol, 1.0 eq.) in DCM (41 mL) was added 1-ethyl-3-(3-dimethylaminopropyl)carbodiimide (EDCI) (1.7 g, 8.98 mmol, 1.1 eq.), and propargylamine (624 μL, 8.2 mmol, 1.0 eq.) at 0° C. and the reaction mixture was stirred for 1 h. The reaction was quenched with 1 M aqueous HCl, diluted with DCM, and the organic layer was separated. The aqueous layer was extracted with DCM. The organic layers were combined, washed with saturated aqueous NaHCO₃, dried over MgSO₄ and concentrated in vacuo. The crude product was purified by silica gel column chromatography (hexane:ethyl acetate:DCM=7:2:1 to 7:3:1, v/v/v) to give Si (1.11 g, 53%). ¹H NMR (600 MHz, CDCl₃) 8.13-8.11 (m, 2H), 8.03-8.01 (m, 2H), 6.51 (s, 1H), 4.28 (dd, J=5.2, 2.6 Hz, 2H), 2.32 (t, J=2.6 Hz, 1H). ¹³C NMR (150 MHz, CDCl₃) δ 165.02, 146.73, 139.98, 128.62, 127.55, 78.76, 72.72, 30.30. ESI-TOF (HRMS) m/z [M+H]⁺ calculated for C₁₀H₉ClNO₃S 257.9986, found 257.9990.

### 4-((1H-1,2,4-Triazol-1-yl)sulfonyl)-N-(prop-2-yn-1-yl)benzamide, HHS-475

To a solution of compound Si (0.1 g, 0.39 mmol, 1.0 eq.) in anhydrous DCM (1.9 mL, 0.2 M) was added 1,2,4-triazole (1.94 mmol, 5.0 eq.) and N,N-diisopropylethylamine (DIPEA) (124 μL, 0.78 mmol, 2.0 eq.) at 0° C. Then the reaction mixture was stirred at room temperature for overnight. The crude product was directly loaded and purified using silica gel flash column chromatography (acetone/DCM=5:100 to 10:100) to afford HHS-475. Yield: 66%, ¹H NMR (600 MHz, (CD₃)₂CO) δ 9.15 (s, 1H), 8.46 (d, J=5.7 Hz, 1H), 8.25-8.22 (m, 2H), 8.21 (s, 1H), 8.20-8.17 (m, 2H), 4.22 (dd, J=5.5, 2.6 Hz, 2H), 2.70 (t, J=2.6 Hz, 1H). ¹³C NMR (150 MHz, (CD₃)₂CO) δ 165.35, 155.46, 146.70, 141.55, 139.00, 129.62, 129.57, 80.72, 72.37, 29.75. ESI-TOF (HRMS) m/z [M+H]⁺ calculated for C₁₂H₁₁N₄O₃S 291.0546, found 291.0546.

### Example 2

### Biological Methods

### Cell Culture:

Cell lines were grown in 10 cm cell culture treated dishes (Thermo Fisher Scientific, Waltham, Massachusetts, United States of America) at 37° C. with 5% CO₂. DM93 cells were grown in RPMI medium supplemented 10% fetal bovine serum (Omega Scientific, Inc., Tarzana, California, United States of America) and 1% L-glutamine (Fisher Scientific, Hampton, New Hampshire, United States of America). HEK293T cells used for transfection were grown in DMEM medium supplemented with 10% fetal bovine serum and 1% L-glutamine.

### SILAC Cell Culture:

SILAC DM93 cells were grown in 10 cm dishes at 37° C. with 5% CO₂ in either “light” or “heavy” media supplemented with 10% dialyzed fetal bovine serum (Omega Scientific, Inc., Tarzana, California, United States of America), 1% L-glutamine (Fisher Scientific, Hampton, New Hampshire, United States of America), and isotopically labelled L-lysine and L-arginine amino acids. “Light” media was supplemented with 100 μg/mL [¹²C₆¹⁴N₂] L-lysine and 100 μg/mL [¹²C₆¹⁴N₄] L-arginine. “Heavy” media was supplemented with 100 μg/mL [¹³C₆¹⁵N₂] L-lysine and 100 μg/mL [¹³C₆¹⁵N₄] L-arginine. The cells were grown for at least five passages to incorporate the amino acids before being used for proteomics experiments.

### Sample Preparation for LC-MS/MS Analysis:

Cells were grown to 90-95% confluence before being washed once with warm, sterile DPBS (1×, 5 mL), followed by addition of 10 mL of serum-free media (supplemented with 1% L-glutamine) containing either DMSO or SuTEx fragment (in DMSO) was added. Light cells were treated with DMSO vehicle while the heavy cells were treated with DMSO or SuTEx fragment (in DMSO) and incubated for 2 hrs. After 2 hrs, the cells were treated with SuTEx alkyne probe (HHS475, 10 μL of 1000× DMSO stock) and incubated for an additional 2 hrs. The media was aspirated and cells washed with cold PBS (5 mL) and harvested. Cells were spun at 500×g for 5 min at 4° C. followed by removal of supernatant. Pellets were resuspended in 1 mL of cold DPBS and spun at 1,400×g for 3 min at 4° C. followed by removal of supernatant. The cells were lysed in PBS by sonication and fractionated (100,000×g, 45 min, 4° C.).The light and heavy soluble fractions were diluted to 2.3 mg/mL in PBS and 432 μL were used for analysis (1 mg total protein). The probe-modified proteomes were conjugated to desthiobiotin-PEG₃-azide (10 μl of 10 mM stock in DMSO; 200 μM final concentration) using TCEP (10 μl of fresh 50 mM stock in water, 1 mM final concentration), TBTA ligand (33 μl of a 1.7 mM 4:1 t-butanol/DMSO stock, 100 μM final concentration) and CuSO₄ (10 μl of 50 mM stock, 1 mM final concentration). Samples were vortexed to mix and incubated for 1 hr at room temperature. The heavy and light proteomes were mixed 1:1 and the subsequent steps, including LC-MS/MS data analysis, were performed as previously described^{[8a]}.

### Molecular Biology:

Plasmid constructs (human protein) were purchased from GenScript (GenScript USA Inc., Piscataway, New Jersey, United States of America): pcDNA 3.1-PTGR2-FLAG and pcDNA 3.1-FAH-FLAG. Plasmids were amplified by transforming XL-1 Blue E. coli through electroporation with 10 ng of plasmid. Transformed bacteria was grown in terrific broth (TB, 1 mL) for 45 minutes at 37° C. An aliquot of this bacteria was plated onto agar bacterial growth plates that had been made with carbenicillin (100 μg/mL), and plates were incubated at 37° C. for 14-18 hrs. Plates were stored at 4° C. and wrapped in parafilm for no longer than one month for use. Single bacterial colonies were scraped from the agar plates and grown in Falcon tubes with terrific broth (5 mL) supplemented with carbenicillin (100 μg/mL) at 37° C. for 14-18 hrs while shaking. Plasmid was extracted and purified using a purification kit sold under the tradename QIAPREP® Spin Miniprep Kit (Qiagen GMBH, Zeeland, Michigan, United States of America) according to manufacturer protocols and stored at −80° C. until needed.

### Gel-Based Chemical Proteomics:

HEK293T cells at 30-50% confluency were transfected with 2.6 μg of FAH or PTGR2 plasmid DNA in a serum-free media/polyethyleneimine solution for 48 hrs. The cells were washed once with warm, sterile DPBS (1×, 5 mL), and then 10 mL of serum-free media (supplemented with 1% L-glutamine) containing either DMSO or SuTEx fragment (in DMSO) was added. After 2 hrs, the cells were treated with 100 μM of SuTEx alkyne probe (HHS-475, 10 μL of 1000× DMSO stock) and incubated for an additional 2 hrs. The media was aspirated and cells washed with cold PBS (5 mL) and harvested. Cells were spun at 500×g for 5 min at 4° C. and supernatant was removed. Pellets were resuspended in 1 mL of cold DPB S and spun at 1,400×g for 3 min at 4° C. and supernatant was removed. The cells were lysed in PBS by sonication and fractionated (100,000×g, 45 min, 4° C.). The soluble fraction was diluted to 1 mg/mL in PBS and 49 μL was used for analysis. The probe-modified proteomes were conjugated to Rhodamine-azide (1 μl of 1.25 mM stock in DMSO) using TCEP (1 μl of fresh 50 mM stock in water), TBTA ligand (3 μl of a 1.7 mM 4:1 t-butanol/DMSO stock,) and CuSO₄ (1 μl of 50 mM stock) and incubated for 1 hr at room temperature. The reaction was quenched with 17 μL of 4×SDS-PAGE loading buffer+βME and vortexed to mix. The samples were analyzed by SDS-PAGE (30 μL) and imaged by in-gel fluorescence scanning.

### Western Blot Analysis:

Western blot analysis of recombinant protein expression of FAH and PTGR2 was performed as previously described^{[22]}.

### PTGR2 Enzymatic Assay:

PTGR2 enzymatic assay was performed based on a previously described chromogenic method^{[17,81]}. HEK293T cells expressing recombinant PTGR2 were treated with DMSO vehicle or indicated concentrations of SuTEx ligand for 2 hrs. The cells were washed with cold PBS twice and harvested. The proteomes were lysed in PBS by sonication and fractionated (100,000×g, 45 min, 4° C.). Soluble proteome (0.5 mg) was incubated for 30 min at 37° C. with 1 mM of EDTA, DTT, NADPH and 20 μM 15-Keto PGE₂ in 0.1 M Tris-HCl (PH 7.5) (final volume of 230 μL) and the substrate consumed was determined by adding 20 μL 20 N NaOH, mixing and measuring absorbance at 500 nm after 5 min.

### Computational Calculations of Molecular Properties

A digital compound library was built up through acquiring SMILES of all the compounds from ChemDraw. All computational calculations from R packages are based on the RStudio platform. Rcpi package^{[23]} was used to calculate physical properties including the number of hydrogen-bond acceptors, the number of hydrogen-bond donors, calculated log P value, molecular refractivity, the number of fluorine atoms, and topological polar surface area. Hybridization ratio and IMF score were acquired through BioMedR package^{[24]}. The number of rotational bonds (rotors) was generated via Open Babel GUI software^{[25]}.

### Example 3

### Fragment-Based Ligand Discovery in Live Cells

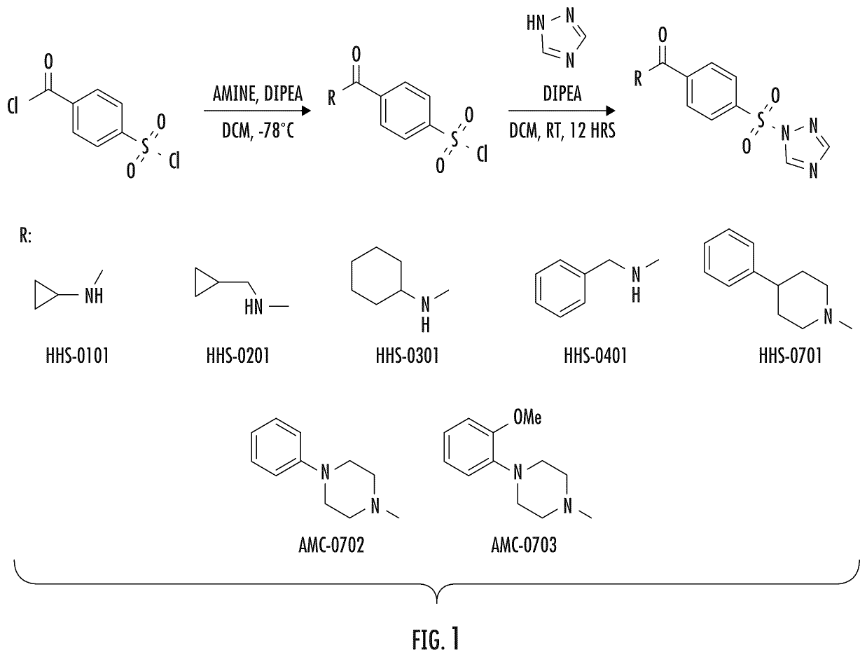

In previous studies, a SuTEx ligand JWB-198 showed cellular activity against the GSTP1 Y8 site^{[10a]}. According to an aspect of the presently disclosed subject matter, analyses were expanded by performing SuTEx compound screening directly in cells to identify additional cell-active protein inhibitors. A focused set of fragment-like 1,2,4-sulfonyl-triazoles were synthesized by coupling substituted amines (cyclopropylamine, cyclopropylmethylamine, cyclohexylamine, benzylamine, and 4-phenylpipieridine) to 4-(chlorosulfonyl) benzoyl chloride followed by addition of an unsubstituted 1,2,4-triazole group. See Example 1 (e.g., Scheme 1 above). See also FIG. 1, synthetic scheme and top row of R groups. These SuTEx fragment ligands were selected for testing because of differences in chemical properties compared with a previous SuTEx fragment ligand collection^{[10a]}. For example, the ligands prepared in Example 1 compounds are enriched for sp³-hybridized atoms. See Table 1, below.

TABLE 1
SuTEx Ligand Chemical Property Comparisons

| | Ref. 10a Ligand | Example 1 Compound |
|---|---|---|
| Property | Averages | Averages |
| HBA | 4.9 | 6.0 |
| HBD | 0.0 | 0.8 |
| logP | 3.5 | 2.7 |
| MR | 81.7 | 84.5 |
| nF | 0.9 | 0.0 |
| TPSA | 86.6 | 100.6 |
| HybRatio | 0.1 | 0.3 |
| FMF | 0.8 | 0.9 |
| rotors | 3.9 | 5.4 |

Key: HBA: hydrogen bond acceptors; HBD: hydrogen bond donors; logP: clogP Octanol/water partition coefficient; MR: molar refractivity; nF: fluorine atoms; TPSA: total polar surface area; HybRatio: hybridization ratio (sp3 carbon centers/(sp2 + sp3 carbon centers)); FMF: fraction of molecular framework2; rotors: rotatable bonds.

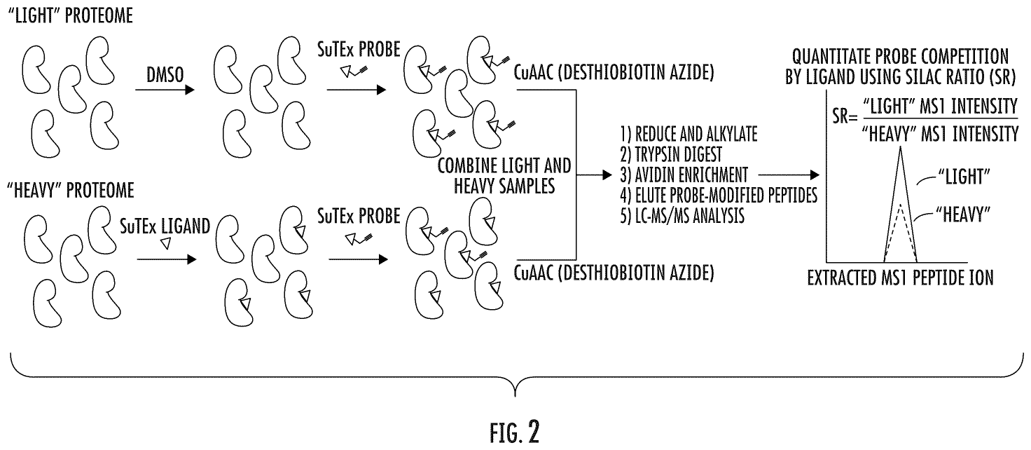

Coupling cell-based screens with chemical proteomics can facilitate the identification of target protein and binding sites that engage and are irreversibly modified by covalent ligands^{[12]}. In these assays, covalent ligands block SuTEx activity-based probe (ABP) labeling at protein sites, and the competition of probe labeling can be used to quantify potency and selectivity across the proteome^{[10a]}. To facilitate quantitative chemical proteomics, DM93 melanoma cells were cultured using stable isotopic labeling by amino acids in cell culture (SILAC)^{[13]}. Cells cultured in “light” media were treated with dimethyl sulfoxide (DMSO) vehicle while cells cultured in “heavy” media were treated with SuTEx ligand (25 μM, 2 hours, 37° C.). Next, cells were treated with HHS-475 probe (100 μM, 2 hours, 37° C.) followed by lysis and processing of proteomes for analysis. Probe-modified proteins from the soluble fraction were conjugated to a desthiobiotin-azide tag by copper-catalyzed azide-alkyne cycloaddition (CuAAC)^{[14]} followed by mixing of the light and heavy proteomes. The combined proteomes were digested with trypsin protease to produce probe-modified peptides, followed by enrichment and purification by avidin affinity chromatography. Probe-modified peptides were resolved, identified, and quantified by high-resolution liquid chromatography-tandem mass spectrometry (LC-MS/MS). See FIG. 2. Resultant data were analyzed as previously described^{[8a, 10a]}. Data from LC-MS chemical proteomic studies are included in Table 2, below. Table 2, below, lists sites of modification targeted by sulfonyl-triazole compounds HHS-0101, HHS-0201, HHS-0301, HHS-0401, and HHS-0701. The format of the table is as follows: protein Uniprot accession number (see uniprot.org); tyrosine site (amino acid position) modified; treatment (sulfonyl-triazole compound identification number, followed by Y (where if multiple compounds modify the same site, the compounds are separated by vertical lines).

### Features of Protein Sites Liganded by SuTEx Ligands:

SuTEx ligands were screened across independent biological replicates (n=3). Probe-modified peptides were subjected to quality control metrics including identification in several biological replicates, Byonic score^{[15]}, and mass accuracy. Quantification of ligand inhibitory activity was evaluated by the ratio of peptide abundances of light-(DMSO-treated) to heavy-(SuTEx fragment-treated) precursor ions. See FIG. 2.

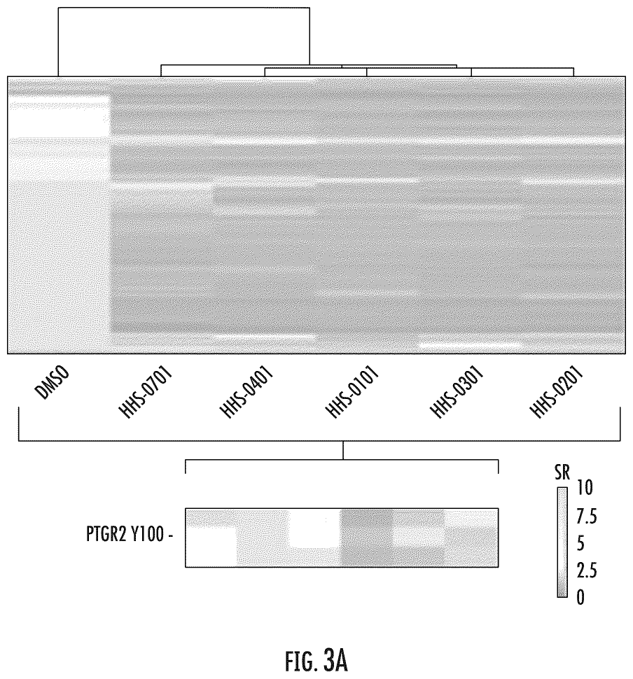

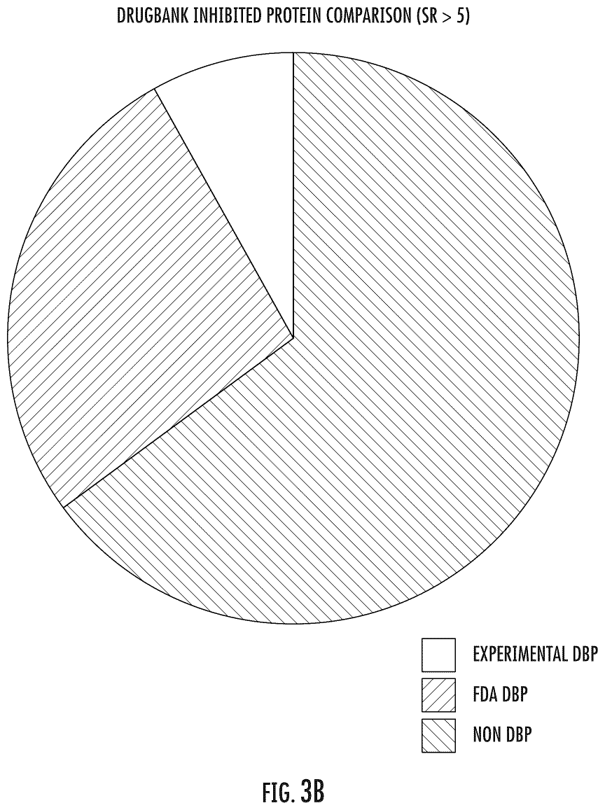

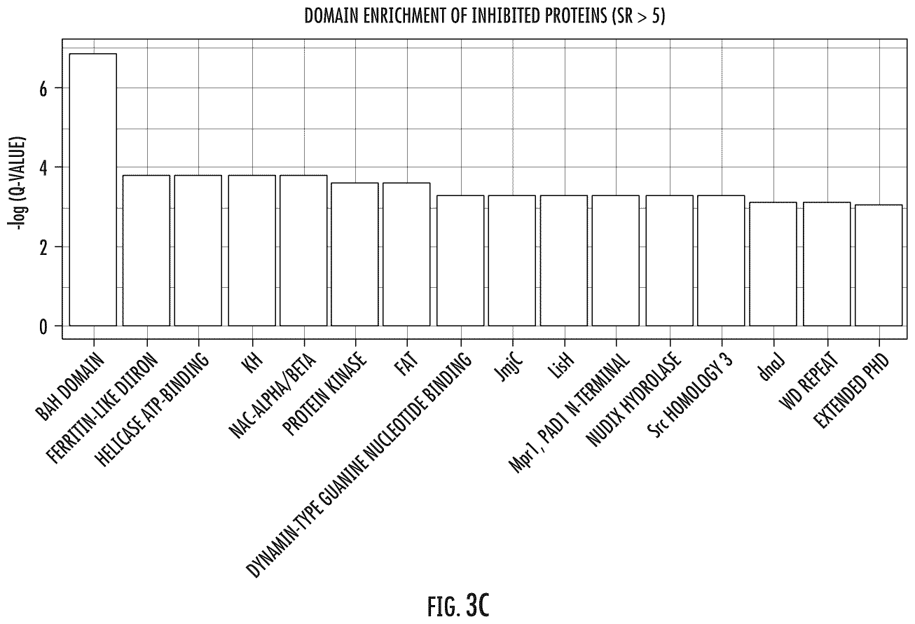

To gain a broader understanding of SuTEx ligand activity and selectivity, a heatmap was generated to visualize overall ligand reactivity against the proteome and also to identify tyrosine sites that showed >80% reductions (SR >5) in probe labeling in cells treated with compounds (liganded sites). See FIG. 3A. The majority of these liganded sites were found on proteins without a corresponding match in the DrugBank database, which highlights the potential for tuning such ligands to target proteins that lack pharmacological probes (Non DBP group). See FIG. 3B. See also Table 3, below. Liganded tyrosine sites were enriched for functional domains including BAH (Uniprot ID PRU00370), ferritin-like diiron (Uniprot ID PRU00085), helicase ATP-binding (Uniprot ID PRU00541), KH (Uniprot ID PRU00117), and NAC-alpha/beta (Uniprot ID PRU00507).

See FIG. 3C. See also Table 4, below. Overall, the data show that SuTEx compounds combined with chemical proteomics can be used to identify cell-active ligands of diverse proteins.

TABLE 2
Liganded Sites from LC-MS Proteomics Study

| Uniprot ID | Site | Treatment |
|---|---|---|
| A6NKT7 | 1008 | HHS0101.Y |
| O00232 | 20 | HHS0101.Y\|HHS0301.Y\|HHS0401.Y\|HHS0701.Y |
| O00267 | 249 | HHS0701.Y |
| O00571 | 266 | HHS0401.Y |
| O14578 | 818 | HHS0301.Y |
| O14777 | 310 | HHS0701.Y |
| O15318 | 43 | HHS0301.Y\|HHS0401.Y |
| O15318 | 56 | HHS0301.Y |
| O15523 | 264 | HHS0401.Y |
| O43156 | 51 | HHS0101.Y\|HHS0401.Y\|HHS0701.Y |
| O43172 | 31 | HHS0101.Y\|HHS0301.Y |
| O43172 | 32 | HHS0101.Y\|HHS0301.Y |
| O43390 | 578 | HHS0201.Y\|HHS0301.Y\|HHS0401.Y\|HHS0701.Y |
| O43660 | 82 | HHS0201.Y |
| O43776 | 45 | HHS0101.Y |
| O43823 | 436 | HHS0401.Y |
| O43852 | 294 | HHS0201.Y |
| O60506 | 570 | HHS0201.Y\|HHS0301.Y\|HHS0401.Y\|HHS0701.Y |
| O60763 | 718 | HHS0201.Y |
| O60828 | 209 | HHS0101.Y\|HHS0401.Y |
| O75131 | 203 | HHS0401.Y |
| O75150 | 258 | HHS0301.Y |
| O75150 | 928 | HHS0101.Y |
| O75351 | 47 | HHS0401.Y |
| O75521 | 285 | HHS0201.Y\|HHS0301.Y\|HHS0401.Y |
| O94925 | 145 | HHS0101.Y |
| O94925 | 304 | HHS0101.Y |
| O95163 | 957 | HHS0101.Y\|HHS0301.Y\|HHS0401.Y |
| O95260 | 63 | HHS0401.Y\|HHS0701.Y |
| O95373 | 416 | HHS0701.Y |
| O95373 | 455 | HHS0701.Y |
| O95453 | 161 | HHS0401.Y |
| O95757 | 639 | HHS0101.Y\|HHS0301.Y\|HHS0401.Y |
| P02794 | 13 | HHS0201.Y |
| P04040 | 358 | HHS0101.Y\|HHS0201.Y\|HHS0301.Y\|HHS0701.Y |
| P05455 | 188 | HHS0401.Y |
| P06132 | 164 | HHS0401.Y |
| P06396 | 469 | HHS0701.Y |
| P06400 | 91 | HHS0301.Y\|HHS0701.Y |
| P07814 | 864 | HHS0201.Y |
| P08238 | 430 | HHS0101.Y\|HHS0201.Y\|HHS0301.Y\|HHS0401.Y |
| P09874 | 645 | HHS0101.Y |
| P09960 | 379 | HHS0701.Y |
| P09960 | 384 | HHS0701.Y |
| P11216 | 204 | HHS0201.Y |
| P11216 | 473 | HHS0101.Y |
| P12081 | 168 | HHS0701.Y |
| P12081 | 172 | HHS0701.Y |
| P12268 | 233 | HHS0401.Y |
| P13010 | 295 | HHS0201.Y |
| P13674 | 250 | HHS0101.Y |
| P14550 | 135 | HHS0401.Y |
| P15559 | 76 | HHS0401.Y\|HHS0701.Y |
| P15880 | 250 | HHS0301.Y |
| P16930 | 244 | HHS0101.Y\|HHS0201.Y\|HHS0301.Y\|HHS0401.Y\|HHS0701.Y |
| P17066 | 373 | HHS0301.Y |
| P18206 | 100 | HHS0201.Y\|HHS0301.Y |
| P18669 | 119 | HHS0401.Y |
| P19174 | 472 | HHS0201.Y |
| P20618 | 135 | HHS0301.Y |
| P20839 | 233 | HHS0401.Y |
| P22059 | 764 | HHS0201.Y\|HHS0701.Y |
| P22626 | 244 | HHS0201.Y |
| P22626 | 247 | HHS0201.Y |
| P22626 | 262 | HHS0101.Y |
| P22626 | 283 | HHS0201.Y |
| P23588 | 258 | HHS0101.Y |
| P23921 | 485 | HHS0401.Y |
| P24534 | 18 | HHS0201.Y\|HHS0401.Y\|HHS0701.Y |
| P24666 | 88 | HHS0201.Y\|HHS0401.Y\|HHS0701.Y |
| P25205 | 147 | HHS0301.Y |
| P25205 | 159 | HHS0701.Y |
| P25205 | 188 | HHS0701.Y |
| P25685 | 52 | HHS0301.Y |
| P25789 | 19 | HHS0101.Y |
| P25789 | 66 | HHS0101.Y\|HHS0201.Y\|HHS0301.Y |
| P26358 | 367 | HHS0401.Y |
| P26599 | 430 | HHS0101.Y\|HHS0201.Y\|HHS0401.Y |
| P28482 | 113 | HHS0101.Y |
| P30086 | 106 | HHS0301.Y |
| P30533 | 336 | HHS0301.Y\|HHS0701.Y |
| P31948 | 134 | HHS0301.Y |
| P35269 | 124 | HHS0701.Y |
| P36507 | 244 | HHS0401.Y |
| P39019 | 54 | HHS0201.Y |
| P40939 | 343 | HHS0701.Y |
| P42704 | 1365 | HHS0201.Y |
| P43490 | 188 | HHS0701.Y |
| P46013 | 340 | HHS0401.Y\|HHS0701.Y |
| P46940 | 977 | HHS0201.Y\|HHS0301.Y\|HHS0701.Y |
| P48147 | 473 | HHS0201.Y |
| P49585 | 173 | HHS0301.Y |
| P49721 | 134 | HHS0701.Y |
| P49792 | 1983 | HHS0101.Y |
| P50502 | 213 | HHS0201.Y |
| P50749 | 270 | HHS0701.Y |
| P50851 | 2656 | HHS0401.Y |
| P51003 | 327 | HHS0401.Y\|HHS0701.Y |
| P51665 | 74 | HHS0701.Y |
| P51784 | 396 | HHS0101.Y |
| P52597 | 210 | HHS0101.Y\|HHS0201.Y\|HHS0701.Y |
| P52597 | 298 | HHS0101.Y\|HHS0701.Y |
| P53621 | 628 | HHS0101.Y\|HHS0401.Y\|HHS0701.Y |
| P53992 | 300 | HHS0301.Y |
| P54578 | 401 | HHS0201.Y |
| P55084 | 402 | HHS0401.Y |
| P55795 | 240 | HHS0701.Y |
| P55795 | 243 | HHS0701.Y |
| P56945 | 267 | HHS0201.Y |
| P57081 | 371 | HHS0401.Y |
| P60900 | 103 | HHS0101.Y\|HHS0201.Y\|HHS0401.Y |
| P61289 | 196 | HHS0101.Y\|HHS0201.Y\|HHS0401.Y |
| P61326 | 34 | HHS0201.Y |
| P61978 | 138 | HHS0701.Y |
| P62263 | 72 | HHS0201.Y\|HHS0301.Y |
| P62277 | 38 | HHS0101.Y |
| P62847 | 81 | HHS0201.Y |
| P62979 | 148 | HHS0401.Y |
| P62993 | 160 | HHS0201.Y |
| P63241 | 127 | HHS0101.Y\|HHS0201.Y\|HHS0701.Y |
| P68104 | 183 | HHS0101.Y |
| P68371 | 50 | HHS0101.Y |
| P78527 | 701 | HHS0301.Y |
| P78537 | 78 | HHS0301.Y\|HHS0401.Y\|HHS0701.Y |
| P82650 | 163 | HHS0701.Y |
| Q00341 | 582 | HHS0201.Y |
| Q00535 | 242 | HHS0201.Y |
| Q01082 | 777 | HHS0101.Y\|HHS0201.Y\|HHS0401.Y |
| Q04323 | 179 | HHS0701.Y |
| Q04637 | 103 | HHS0201.Y\|HHS0301.Y\|HHS0401.Y\|HHS0701.Y |
| Q04637 | 104 | HHS0301.Y\|HHS0701.Y |
| Q04637 | 81 | HHS0301.Y |
| Q04837 | 101 | HHS0701.Y |
| Q04837 | 119 | HHS0701.Y |
| Q06203 | 451 | HHS0301.Y |
| Q09028 | 154 | HHS0301.Y\|HHS0701.Y |
| Q12874 | 139 | HHS0301.Y |
| Q12931 | 559 | HHS0701.Y |
| Q12965 | 9 | HHS0301.Y |
| Q13011 | 149 | HHS0701.Y |
| Q13033 | 137 | HHS0101.Y\|HHS0401.Y |
| Q13057 | 126 | HHS0301.Y |
| Q13107 | 192 | HHS0201.Y\|HHS0301.Y |
| Q13151 | 180 | HHS0101.Y |
| Q13330 | 22 | HHS0301.Y |
| Q13428 | 15 | HHS0401.Y |
| Q13535 | 2067 | HHS0701.Y |
| Q14157 | 204 | HHS0201.Y |
| Q14157 | 214 | HHS0201.Y |
| Q14157 | 803 | HHS0401.Y |
| Q14247 | 154 | HHS0301.Y\|HHS0401.Y |
| Q14566 | 783 | HHS0201.Y |
| Q14694 | 359 | HHS0301.Y |
| Q14847 | 57 | HHS0101.Y |
| Q14914 | 262 | HHS0401.Y |
| Q14978 | 289 | HHS0101.Y\|HHS0701.Y |
| Q14980 | 1836 | HHS0401.Y |
| Q15020 | 541 | HHS0101.Y\|HHS0201.Y\|HHS0301.Y\|HHS0401.Y\|HHS0701.Y |
| Q15154 | 1424 | HHS0401.Y\|HHS0701.Y |
| Q15293 | 320 | HHS0101.Y |
| Q15654 | 157 | HHS0201.Y |
| Q16576 | 153 | HHS0301.Y\|HHS0701.Y |
| Q16836 | 264 | HHS0201.Y\|HHS0401.Y\|HHS0701.Y |
| Q5T6F2 | 823 | HHS0401.Y |
| Q5T6F2 | 825 | HHS0401.Y |
| Q5VTE0 | 183 | HHS0101.Y |
| Q6IS14 | 127 | HHS0101.Y\|HHS0201.Y\|HHS0701.Y |
| Q6N069 | 86 | HHS0101.Y\|HHS0701.Y |
| Q6NZY4 | 258 | HHS0701.Y |
| Q6ZSZ5 | 1012 | HHS0201.Y |
| Q7Z460 | 1267 | HHS0101.Y\|HHS0301.Y\|HHS0401.Y |
| Q7Z6E9 | 218 | HHS0301.Y |
| Q7Z6M1 | 87 | HHS0401.Y\|HHS0701.Y |
| Q86VS8 | 323 | HHS0101.Y\|HHS0401.Y\|HHS0701.Y |
| Q86XP3 | 685 | HHS0201.Y |
| Q8IWS0 | 303 | HHS0201.Y |
| Q8IX04 | 158 | HHS0101.Y\|HHS0201.Y\|HHS0301.Y |
| Q8N0Y7 | 119 | HHS0401.Y |
| Q8N6H7 | 445 | HHS0701.Y |
| Q8N8N7 | 100 | HHS0701.Y |
| Q8N8N7 | 265 | HHS0401.Y |
| Q8NBF2 | 23 | HHS0101.Y |
| Q8NFI4 | 213 | HHS0201.Y |
| Q8WVJ2 | 145 | HHS0101.Y |
| Q92620 | 261 | HHS0701.Y |
| Q92734 | 392 | HHS0701.Y |
| Q92804 | 170 | HHS0201.Y\|HHS0301.Y |
| Q92878 | 244 | HHS0201.Y\|HHS0301.Y |
| Q92889 | 344 | HHS0301.Y\|HHS0401.Y |
| Q92900 | 518 | HHS0101.Y\|HHS0201.Y\|HHS0401.Y\|HHS0701.Y |
| Q92900 | 946 | HHS0101.Y |
| Q92974 | 894 | HHS0101.Y\|HHS0401.Y |
| Q93052 | 234 | HHS0301.Y\|HHS0401.Y |
| Q93052 | 251 | HHS0101.Y\|HHS0401.Y |
| Q93052 | 332 | HHS0101.Y |
| Q969T7 | 85 | HHS0101.Y |
| Q969T9 | 232 | HHS0101.Y\|HHS0701.Y |
| Q96A72 | 36 | HHS0201.Y |
| Q96AE4 | 438 | HHS0101.Y |
| Q96AE4 | 619 | HHS0701.Y |
| Q96AT1 | 109 | HHS0401.Y |
| Q96AT1 | 9 | HHS0101.Y |
| Q96BR5 | 72 | HHS0401.Y |
| Q96C86 | 116 | HHS0101.Y\|HHS0401.Y |
| Q99797 | 273 | HHS0201.Y |
| Q9BPW8 | 83 | HHS0701.Y |
| Q9BTC8 | 22 | HHS0301.Y |
| Q9BUJ2 | 111 | HHS0301.Y\|HHS0701.Y |
| Q9BUT1 | 49 | HHS0101.Y |
| Q9BWD1 | 237 | HHS0101.Y\|HHS0201.Y\|HHS0301.Y\|HHS0401.Y |
| Q9BWT3 | 326 | HHS0201.Y\|HHS0701.Y |
| Q9BXJ9 | 86 | HHS0101.Y\|HHS0701.Y |
| Q9BXS6 | 165 | HHS0101.Y |
| Q9C0C2 | 897 | HHS0201.Y\|HHS0301.Y\|HHS0401.Y\|HHS0701.Y |
| Q9H009 | 112 | HHS0101.Y\|HHS0201.Y\|HHS0301.Y\|HHS0401.Y\|HHS0701.Y |
| Q9H223 | 70 | HHS0701.Y |
| Q9H3P2 | 324 | HHS0401.Y\|HHS0701.Y |
| Q9H3P7 | 293 | HHS0201.Y\|HHS0301.Y |
| Q9H788 | 194 | HHS0701.Y |
| Q9HAU0 | 553 | HHS0701.Y |
| Q9HCE5 | 169 | HHS0201.Y\|HHS0301.Y |
| Q9HCH5 | 289 | HHS0101.Y\|HHS0201.Y\|HHS0301.Y\|HHS0401.Y\|HHS0701.Y |
| Q9NUJ1 | 215 | HHS0201.Y\|HHS0701.Y |
| Q9NV35 | 92 | HHS0201.Y |
| Q9NVE7 | 338 | HHS0201.Y\|HHS0401.Y |
| Q9NW64 | 116 | HHS0301.Y |
| Q9NYF8 | 322 | HHS0101.Y\|HHS0201.Y\|HHS0301.Y\|HHS0401.Y |
| Q9NZQ3 | 52 | HHS0301.Y |
| Q9P2B4 | 343 | HHS0701.Y |
| Q9P2E9 | 647 | HHS0301.Y |
| Q9UDY2 | 423 | HHS0201.Y\|HHS0301.Y |
| Q9UFN0 | 42 | HHS0201.Y\|HHS0301.Y\|HHS0401.Y\|HHS0701.Y |
| Q9UI12 | 163 | HHS0201.Y\|HHS0301.Y |
| Q9UJ68 | 211 | HHS0101.Y\|HHS0401.Y\|HHS0701.Y |
| Q9ULV4 | 301 | HHS0301.Y |
| Q9ULV4 | 304 | HHS0701.Y |
| Q9UMX5 | 82 | HHS0701.Y |
| Q9UPP1 | 383 | HHS0201.Y |
| Q9UQ35 | 2390 | HHS0301.Y |
| Q9Y2H2 | 887 | HHS0401.Y\|HHS0701.Y |
| Q9Y2S7 | 168 | HHS0101.Y |
| Q9Y2W1 | 344 | HHS0201.Y\|HHS0301.Y |
| Q9Y520 | 26 | HHS0201.Y |
| Q9Y520 | 2707 | HHS0401.Y |
| Q9Y5K3 | 173 | HHS0301.Y |
| Q9Y5K6 | 361 | HHS0101.Y\|HHS0201.Y\|HHS0401.Y |
| Q9Y6D5 | 1632 | HHS0101.Y\|HHS0301.Y\|HHS0701.Y |
| Q9Y6D5 | 97 | HHS0401.Y |

TABLE 3
DrugBank Search for Liganded Sites

| Protein | Number of Liganded Proteins |
|---|---|
| In_FDA (DBP) | 49 |
| Out_FDA (DBP) | 168 |
| In_all proteins | 68 |
| Out_all proteins | 149 |

TABLE 4
Domain Enrichment Output for Liganded Sites

| Domain Enrichment Output | | | | | | |
|---|---|---|---|---|---|---|
| for Liganded Sites | | | | | | |
| | | Number of | | | | |
| | Number | Proteins | | | | |
| ProRule | of Time | Possible | Probability | | | |
| Number | Liganded | to Ligand | Score | p-value | Domain Name | Q-value |
| PRU00370 | 2 | 13 | 0.000427674 | 5.45E−05 | BAH domain | 0.001035745 |
| PRU00117 | 2 | 87 | 0.002862125 | 0.002352183 | KH domain | 0.02181681 |
| PRU00541 | 2 | 117 | 0.003849064 | 0.004190426 | Helicase ATP-binding domain | 0.02181681 |
| PRU00085 | 1 | 6 | 0.000197388 | 0.004923027 | Ferritin-like diiron domain | 0.02181681 |
| PRU00507 | 1 | 7 | 0.000230286 | 0.005741266 | NAC-A/B (NAC-alpha/beta) domain | 0.02181681 |
| PRU00159 | 3 | 512 | 0.016843767 | 0.008330901 | Protein kinase domain | 0.026381186 |
| PRU00534 | 1 | 12 | 0.000394776 | 0.009822782 | FAT domain | 0.026661836 |
| PRU01182 | 1 | 24 | 0.000789552 | 0.0195529 | MPN (Mpr1, Pad1 N-terminal) domain | 0.038113881 |
| PRU00126 | 1 | 26 | 0.000855348 | 0.021165636 | LisH | 0.038113881 |
| PRU00794 | 1 | 27 | 0.000888246 | 0.021971049 | Nudix hydrolase domain | 0.038113881 |
| PRU01055 | 1 | 28 | 0.000921144 | 0.022775825 | Dynamin-type guanine nucleotide- | 0.038113881 |
| | | | | | binding (G) domain | |
| PRU00538 | 1 | 32 | 0.001052735 | 0.025988579 | JmjC domain | 0.038113881 |
| PRU00192 | 2 | 306 | 0.010066783 | 0.026077919 | Src homology 3 (SH3) domain | 0.038113881 |
| PRU00221 | 1 | 43 | 0.001414613 | 0.034771453 | WD repeat | 0.044043841 |
| PRU00286 | 1 | 43 | 0.001414613 | 0.034771453 | dnaJ domain | 0.044043841 |
| PRU01146 | 1 | 49 | 0.001612001 | 0.039530015 | Extended PHD (ePHD) domain | 0.046941893 |
| PRU00041 | 1 | 204 | 0.006711189 | 0.154937971 | C2 domain | 0.173165968 |
| PRU00176 | 1 | 378 | 0.012435438 | 0.268629509 | RNA recognition motif (RRM) domain | 0.283553371 |
| PRU00448 | 1 | 576 | 0.018949238 | 0.380149464 | EF-hand | 0.380149464 |

### Example 4

### Identifying Tyrosine-Reactive Ligands of FAH and PTGR2

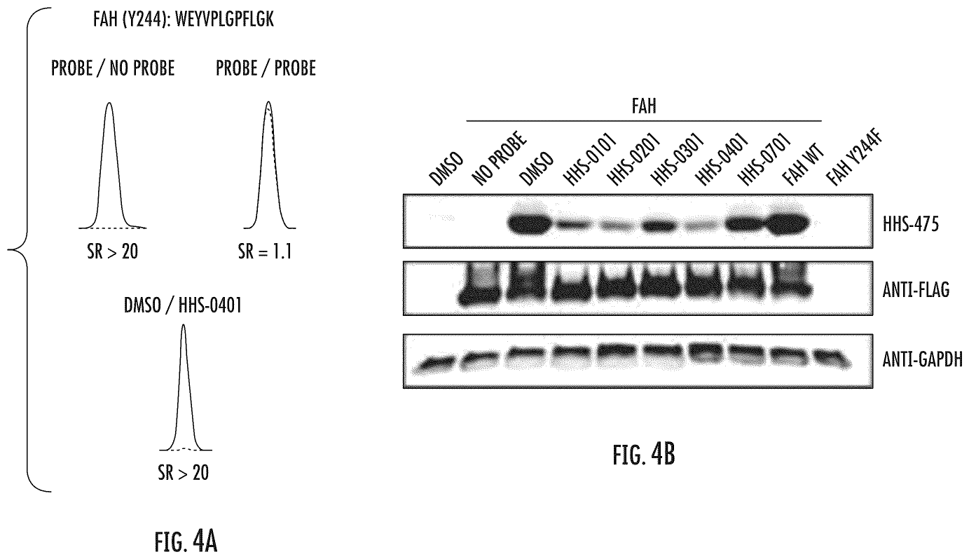

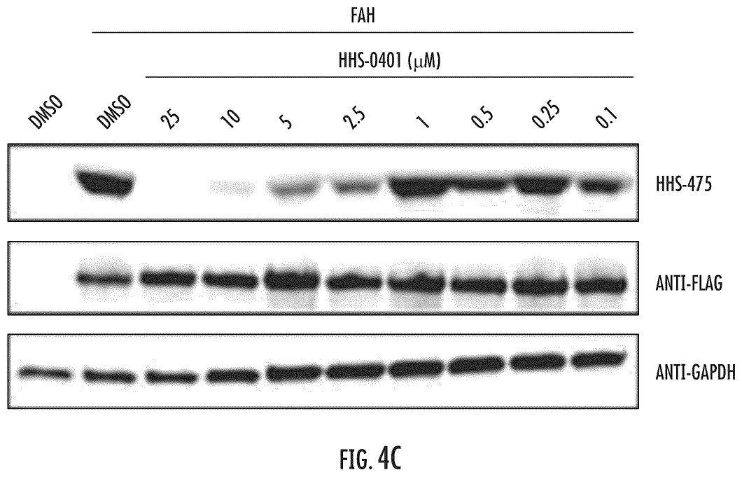

Competitive gel-based chemoproteomic assays were performed using recombinant proteins from select targets to verify our LC-MS findings. Fumarylacetoacetase (FAH) was selected because of its importance in amino acid catabolism^{[16]} and previous identification of a hyper-reactive tyrosine site (Y244)^{[8a]}. Quantitative chemical proteomic studies identified HHS-0401 as a ligand for the Y244 site of FAH. See FIG. 4A. These findings were confirmed by treating HEK293T cells expressing recombinant FAH with SuTEx ligands (25 μM, 2 hours, 37° C.) followed by labeling with HHS-475 probe (100 μM, 2 hours, 37° C.), cell lysis, and conjugation with rhodamine azide by CuAAC to visualize probe-modified proteins using in-gel fluorescence scanning. HHS-0401 and several additional ligands blocked HHS-475 labeling of FAH. See FIG. 4B. Interestingly, mutation of the Y244 site resulted in a mutant FAH protein (Y244F) that could not be recombinantly overexpressed. See FIG. 4B. Finally, it was demonstrated that HHS-0401 is capable of competing probe labeling of recombinant FAH in a concentration-dependent manner in live cells. See FIG. 4C.

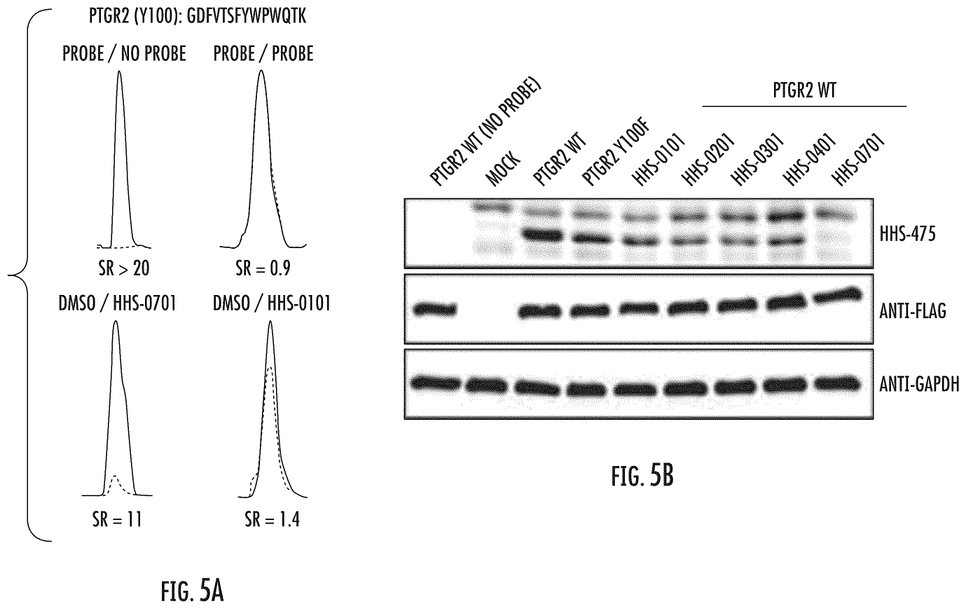

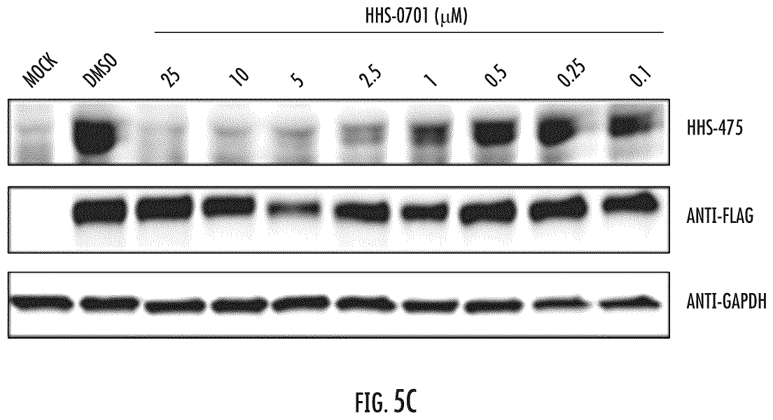

Confirmatory studies were also performed on prostaglandin reductase 2 (PTGR2) because of its role in the reduction of the bioactive lipid, 15-keto-PGE₂^{[17]}. Quantitative chemical proteomics demonstrated that PTGR2 Y100 is liganded by HHS-0701, but not HHS-0101, under the same treatment conditions (SILAC ratio or SR=11 and 1.4, respectively). See FIG. 5A. The chemical proteomic studies also identified additional tyrosine sites (e.g. Y265) competed by HHS-0701 albeit to a lesser degree (SR<5) compared with Y100. See Table 2, above. The LC-MS findings were verified by using gel-based chemical proteomics to demonstrate that treatment of HEK293T cells overexpressing recombinant PTGR2 with HHS-0701, but not HHS-0101, could block HHS-475 labeling of PTGR2. Expression of recombinant PTGR2 Y100F mutant protein resulted in decreased HHS-475 live-cell probe labeling compared to wild-type PTGR2. See FIG. 5B. The remaining fluorescent signal detected on PTGR2 Y100F mutant protein is likely due to probe labeling of additional tyrosine sites identified on wild-type protein. See Table 2. Treatment of recombinant PTGR2 overexpressing HEK293T cells with varying concentrations of HHS-0701 resulted in concentration-dependent blockade of probe labeling. See FIG. 5C.

In summary, LC-MS findings of liganded sites were confirmed by showing concentration-dependent blockade of probe labeling of recombinant target proteins by candidate ligands, but not structurally analogous negative control counterparts.

### Example 5

### HHS-0701 Inhibits PTGR2 Biochemical Activity

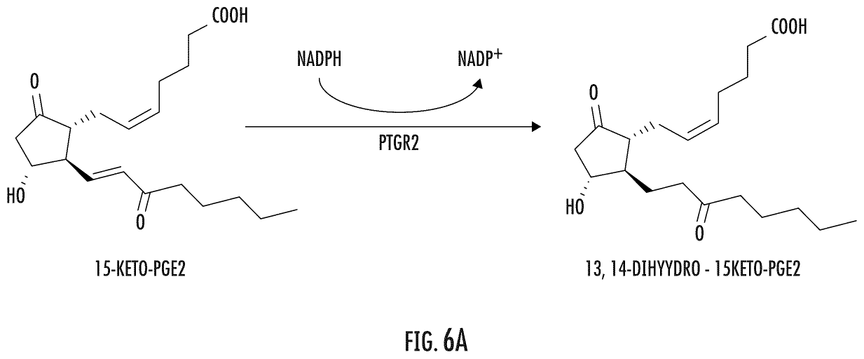

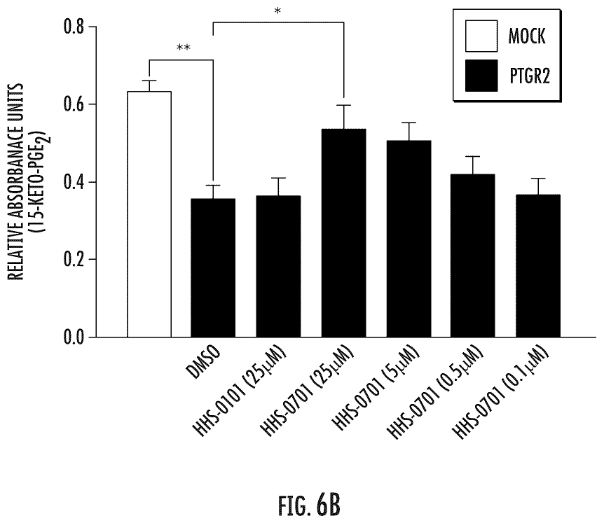

The effect of liganding tyrosine sites on the biochemical function of PTGR2 was investigated. PTGR2 has a role in metabolism and there is an ability to measure its biochemical activity using an established 15-keto-PGE₂ substrate assay^{[17-18]}. See FIG. 6A. In brief, recombinant PTGR2 expressing HEK293T cells were treated with DMSO, HHS-0701, or HHS-0101 (negative control) for 2 hours at 37° C. Cells were lysed and the soluble fractions incubated in an assay buffer containing 15-keto-PGE₂ (20 μM) for 30 minutes at 37° C. After quenching reactions in NaOH, the amount of substrate remaining was quantified by absorbance of a red chromophore that forms in an alkaline solution of 15-keto-PGE₂^{[17-18]}. See also Example 2, above for additional details of the assay). Comparison of non-metabolized substrate between mock- and PTGR2-transfected proteomes was used to determine biochemical activity of recombinant PTGR2, which served as the basis for evaluating inhibitory activity of SuTEx ligands from live cell treatments. See FIG. 6B. A concentration-dependent inhibition of PTGR2 activity by HHS-0701 was observed. No inhibitory activity was observed in proteomes from cells treated with the negative control molecule HHS-0101. See FIG. 6B.

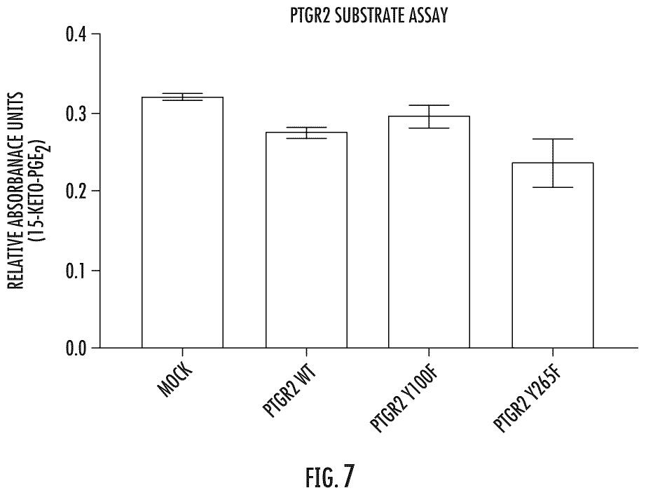

A biochemical assay to evaluate the importance of liganded tyrosine sites for PTGR2 catalytic activity. Biochemical activity of proteomes expressing recombinant PTGR2 wild-type, Y100F, and Y265F proteins was compared. Opposing effects on PTGR2 activity were found from mutations at the Y100 compared with Y265 site. The PTGR2 Y100F mutant showed decreased biochemical activity while the Y265F mutant protein showed comparable and potentially a mild increase in catalytic activity. See FIG. 7. Collectively, these results demonstrate that SuTEx ligands serve as live-cell-active inhibitors of PTGR2 biochemical activity. Mutagenesis studies support a biochemical role for liganded tyrosine sites on PTGR2.

### Example 6

### Discussion of Examples 1-5

Sulfonyl-triazoles are a promising scaffold for developing global tyrosine-reactive probes and protein-targeted ligands^{[19]}. Herein is demonstrated the utility of the SuTEx platform for discovery of cell-active ligands for modulating protein function. The present studies involve the use of a SuTEx ligand library enriched for sp³-hybridized atoms. See Table 1, above.

A series of 1,2,4-sulfonyl-triazoles was synthesized exhibiting functional group diversity on the adduct group (AG) and their SAR for liganding tyrosine sites on proteins directly in live cells was evaluated. See FIG. 2, top row of R groups. Chemoproteomic evaluations revealed that liganded sites were enriched for functional protein domains on well-studied and target proteins that lacked pharmacological probes. Follow-up studies on recombinant proteins verified the ability of SuTEx ligands to target orthogonal proteins and, in the case of PTGR2, serve as inhibitors of biochemical function. See FIG. 6B.

Using PTGR2 as a model example, the present studies show that targeting tyrosine sites in live cells can affect the biochemical function of this lipid enzyme. PTGR2 catalyzes the NADPH-dependent reduction of 15-keto-PGE₂, an endogenous ligand for the nuclear receptor PPARγ, to 13,14-dihydro-15-keto-PGE₂^{[20]}. PTGR2 has been found to be expressed in pancreatic cancer tissues but is absent in normal counterparts. Knockdown of PTGR2 was found to reduce tumor growth and induce apoptosis through ROS-mediated signaling involving ERK 1/2 and caspase 3 activities^{[21]}. Given the signaling role of 15-keto-PGE₂ in key lipid pathways^{[20]}, potent and selective PTGR2 inhibitors represent potential therapeutic compounds.

### Example 7

### PTGR2 Inhibitor Analogs AMC-0702 and AMC-0703

To a solution of 4-(chlorosulfonyl) benzoyl chloride (2.1 mmol, 1.0 eq.) in anhydrous DCM was added phenylpiperazine or 1-(2-methoxyphenyl)piperazine (2.1 mmol, 1.0 eq.) and N,N-diisopropylethylamine (DIPEA) (2.3 mmol, 1.1 eq.) at −78° C. The reaction mixture was slowly warmed up to 0° C., and then to the reaction mixture was added the 1,2,4-triazole (6.3 mmol, 3.0 eq.) and N,N-diisopropylethylamine (DIPEA) (2.3 mmol, 1.1 eq.) at −15° C. Then the reaction mixture was warmed up to room temperature and stirred overnight. The crude products were washed with brine and extracted with DCM, dried with sodium sulfate, collected via vacuum filtration, and concentrated. They were then redissolved in minimal DCM, and loaded onto a 25 g silica chromatography column sold under the tradename BIOTAGE® Sfar HCD (Biotage AB, Uppsala, Sweden) and eluted using a DCM/Acetone gradient (starting at 0% acetone and progressing to 2%, then 5%, then 10%) using a chromatography system sold under the tradename BIOTAGE® ISOLARA™ One (Biotage AB, Uppsala, Sweden). Product was concentrated and recrystallized in hot ethyl acetate and n-heptane, collected via vacuum filtration, and concentrated to yield AMC-0702 and AMC-0703.

The structures of AMC-0702 and AMC-0703 are show in FIG. 8A. The molecular weights of AMC-0702 and AMC-0703 are 397.4530 and 427.4790, respectively. High resolution mass spectroscopy (HRMS) results for AMC-0702: [M+H]⁺ calculated 398.1287, found 398.1283. HRMS results for AMC-0703: [M+H]⁺ calculated 428.1392, found 428.1387.

To assess the PTGR2 inhibitory activity of AMC-0702 and AMC-0703, HEK293T cells expressing PTGR2 were pretreated with vehicle, HHS-0101 (25 μM), HHS-0701 (25 μM), AMC-0702 (25 μM-0.1 μM), or AMC-0703 (25 μM-0.1 μM) for 2 hours followed by HHS-475 probe labelling in live cells (25 μM, 2 hours). Cells were lysed and probe-modified PTGR2 in proteomes detected by copper-catalyzed azide-alkyne cycloaddition conjugation of rhodamine-azide, SDS-PAGE, and in-gel fluorescence scanning. Inhibitory activity of compounds is determined by reductions in fluorescent band intensity from HHS-475 probe labeling of proteins detected in proteomes. See Western blot analysis was used to confirm equivalent expression of recombinant PTGR2 (anti-FLAG) and equivalent protein loading of samples (anti-GAPDH). FIG. 8B is a composite image of gel-based proteomics analysis showing that AMC-0703 had the highest potency against PTGR2.

### Example 8

### Synthesis of Additional Exemplary Sulfonyl-Triazole PTGR2 Inhibitors and/or Probes

Sulfonyl-triazole PTGR2 inhibitors and/or probes were prepared by reacting various sulfonyl chlorides or sulfonyl fluorides with a 1,2,4-triazole or a substituted 1,2,4-triazole according to one of Procedures A-C.

### Procedure A.

A flame-dried 6-dram vial fixed with a stir bar was charged with sulfonyl chloride (0.24 mmol) and purged with N2 via Schlenk technique. Dry THE (48 mL) was added and the reaction stirred. DIEA (0.26 mmol) was added followed by triazole (0.26 mmol). The reaction was stirred at room temperature overnight. The reaction mixture was concentrated in vacuo and purified by flash chromatography (10-40% gradient ethyl acetate in hexanes).

### Procedure B.

A flame-dried 6-dram vial fixed with a stir bar was charged with triazole (0.41 mmol) and purged with N2 via Schlenk technique. Dry THF (0.40 mL) was added and the reaction stirred and cooled to 0° C. with an ice/water bath. After cooling for 10 minutes, sodium hydride (0.41 mmol, 60% in parrafin oil) was added. The reaction was stirred at 0° C. for 30 minutes. The sulfonyl chloride (0.37 mmol) was dissolved in dry THF (0.40 mL). The sulfonyl chloride solution was added to the triazolide solution and the reaction slowly warmed to room temperature overnight. The reaction mixture was concentrated in vacuo and purified by flash chromatography (10-40% gradient ethyl acetate in hexanes).

### Procedure C.

Sulfonyl fluoride (0.27 mmol), triazole (0.30 mmol), and potassium carbonate (0.30 mmol) were added to a 2-dram vial. Water (0.54 mL) was added and the reaction stirred overnight. About 2 mL of water was added to the reaction. The aqueous layer was extracted three times with ethyl acetate (5 mL each time). The combined organic layers was dried over sodium sulfate and concentrated in vacuo. The crude residue was purified by flash chromatography (10-40% gradient ethyl acetate in hexanes).

### Characterization of Sulfonyl-Triazole PTGR2 Inhibitors:

### (4-(2-methoxyphenyl)piperazin-1-yl)(4-((3-phenyl-1H-1,2,4-triazol-1-yl)-sulfonyl)phenyl)methanone (RJG-1101)

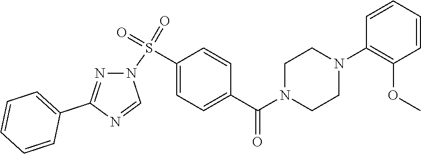 [SMILES: COc1ccccc1N1CCN(C(=O)c2ccc(S(=O)(=O)n3cnc(-c4ccccc4)n3)cc2)CC1]

Prepared using Procedure A. White solid (17.9 mg, 42%). ¹H NMR (600 MHz, cdcl₃) δ 8.75 (s, 1H), 8.20-8.17 (m, 2H), 8.14-8.06 (m, 3H), 7.66-7.62 (m, 2H), 7.45-7.41 (m, 4H), 7.03 (d, J=8.2 Hz, 1H), 6.94-6.89 (m, 1H), 6.87 (d, J=8.1 Hz, 1H), 3.96 (s, 3H), 3.84 (s, 4H), 3.50 (s, 3H), 3.14 (s, 3H), 2.98 (s, 3H). ¹³C NMR (151 MHz, cdcl₃) δ 167.72, 160.81, 154.69, 144.91, 142.82, 136.98, 130.21, 129.30, 128.46, 109.69, 103.67, 55.39, 50.12, 49.67, 47.47, 42.22. HRMS (ESI-TOF) m/z: [M+H]⁺ Calcd for C₂₆H₂₆N₅O₄S⁺, 504.1700, Found, 504.1703.

### (4-((1H-1,2,4-triazol-1-yl)sulfonyl)phenyl)(4-(3-methoxyphenyl)piperazin-1-yl)methanone (RJG-1103)

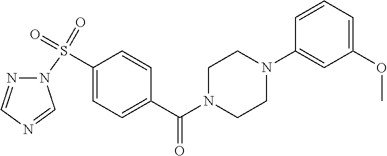 [SMILES: COc1cccc(N2CCN(C(=O)c3ccc(S(=O)(=O)n4cncn4)cc3)CC2)c1]

Prepared using Procedure A. Off-white solid (14.1 mg, 46%). ¹H NMR (600 MHz, cdcl₃) δ 8.76 (s, 1H), 8.19-8.14 (m, 2H), 8.06 (s, 1H), 7.68-7.62 (m, 2H), 7.22 (t, J=8.1 Hz, 1H), 6.63-6.49 (m, 3H), 3.99 (s, 2H), 3.80 (s, 3H), 3.54 (s, 3H), 3.30 (s, 2H), 3.13 (s, 2H). ¹³C NMR (151 MHz, cdcl₃) δ 167.79, 165.58, 152.36, 145.64, 137.21, 130.83, 129.19, 129.12, 128.85, 128.42, 127.28, 121.29, 111.62, 55.60, 51.27, 50.68, 48.04, 42.45. HRMS (ESI-TOF) m/z: [M+H]⁺ Calcd for C₂₀H₂₂N₅O₄S⁺, 428.1387, Found, 428.1378.

### (4-((1H-1,2,4-triazol-1-yl)sulfonyl)phenyl)(4-(4-methoxyphenyl)piperazin-1-yl)methanone (RJG-1105)

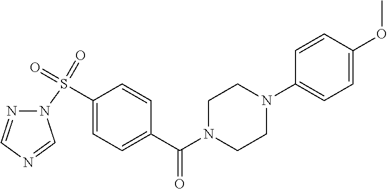 [SMILES: COc1ccc(N2CCN(C(=O)c3ccc(S(=O)(=O)n4cncn4)cc3)CC2)cc1]

Prepared using Procedure A. White solid (11.0 mg, 26%). ¹H NMR (600 MHz, cdcl₃) δ 8.78 (s, 1H), 8.18-8.13 (m, 2H), 8.06 (s, 1H), 7.67-7.63 (m, 2H), 7.00-6.89 (m, 2H), 6.86 (d, J=8.3 Hz, 2H), 3.96 (s, 2H), 3.78 (s, 3H), 3.50 (s, 2H), 3.17 (s, 2H), 3.01 (s, 2H). ¹³C NMR (151 MHz, cdcl₃) δ 167.71, 154.71, 144.97, 142.91, 136.95, 129.29, 128.46, 119.38, 114.77, 55.69, 51.68, 50.94, 47.69, 42.39. HRMS (ESI-TOF) m/z: [M+H]⁺ Calcd for C₂₀H₂₂N₅O₄S⁺, 428.1387, Found, 428.1382.

### (4-((3-(4-bromophenyl)-1H-1,2,4-triazol-1-yl)sulfonyl)phenyl)(4-(2-methoxy-phenyl)piperazin-1-yl)methanone (RJG-1112)

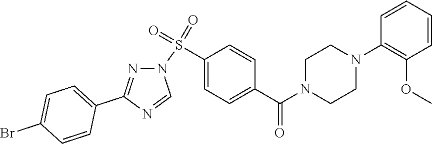 [SMILES: COc1ccccc1N1CCN(C(=O)c2ccc(S(=O)(=O)n3cnc(-c4ccc(Br)cc4)n3)cc2)CC1]

Prepared using Procedure A. White solid (20.4 mg, 39%). ¹H NMR (600 MHz, cdcl₃) δ 8.75 (s, 1H), 8.21-8.16 (m, 2H), 8.00-7.95 (m, 2H), 7.67-7.63 (m, 2H), 7.60-7.54 (m, 2H), 7.08-7.02 (m, 1H), 6.95-6.86 (m, 3H), 3.97 (s, 2H), 3.85 (s, 3H), 3.51 (s, 2H), 3.14 (s, 2H), 2.99 (s, 2H). ¹³C NMR (151 MHz, cdcl₃) δ 167.97, 164.98, 152.60, 145.96, 143.28, 137.25, 132.34, 129.47, 129.03, 128.71, 128.33, 125.56, 121.50, 111.81, 55.83, 51.48, 50.86, 48.26, 42.82. HRMS (ESI-TOF) m/z: [M+H]⁺ Calcd for C₂₆H₂₅BrN₅O₄S⁺, 582.0805, Found, 582.0797.

### (4-((3-(4-fluorophenyl)-1H-1,2,4-triazol-1-yl)sulfonyl)phenyl)(4-(2-methoxy-phenyl)piperazin-1-yl)methanone (RJG-1114)

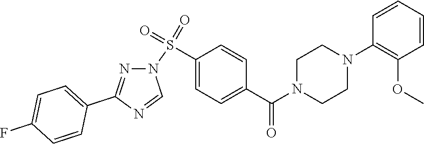 [SMILES: COc1ccccc1N1CCN(C(=O)c2ccc(S(=O)(=O)n3cnc(-c4ccc(F)cc4)n3)cc2)CC1]

Prepared using Procedure A. White solid (26.6 mg, 59%). ¹H NMR (600 MHz, cdcl₃) δ 8.74 (s, 1H), 8.22-8.16 (m, 2H), 8.14-8.07 (m, 2H), 7.68-7.63 (m, 2H), 7.16-7.08 (m, 2H), 7.08-7.03 (m, 1H), 6.95-6.86 (m, 3H), 3.97 (s, 2H), 3.85 (s, 3H), 3.52 (s, 2H), 3.20-3.12 (m, 2H), 3.00 (s, 2H). ¹⁹F NMR (564 MHz, cdcl₃) 6-109.43 (tt, J=8.5, 5.4 Hz). ¹³C NMR (151 MHz, cdcl₃) δ 167.74, 164.74, 164.46 (d, J=256.4 Hz), 152.35, 145.68, 142.96, 137.08, 129.36 (d, J=8.6 Hz), 129.19, 128.43, 125.37, 121.25, 115.96 (d, J=22.0 Hz), 111.57, 55.57, 51.23, 50.62, 42.52. HRMS (ESI-TOF) m/z: [M+H]⁺ Calcd for C₂₆H₂₅FN₅O₄S⁺, 522.1606, Found, 522.1598.

### (4-(2-methoxyphenyl)piperazin-1-yl)(4-((3-(4-(trifluoromethyl)phenyl)-1H-1,2,4-triazol-1-yl)sulfonyl)phenyl)methanone (RJG-1115)

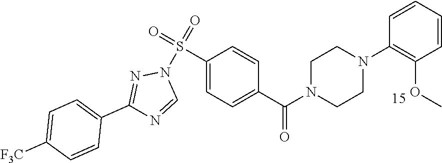 [SMILES: COc1ccccc1N1CCN(C(=O)c2ccc(S(=O)(=O)n3cnc(-c4ccc(C(F)(F)F)cc4)n3)cc2)CC1]

Prepared using Procedure A. White solid (26.8 mg, 54%). ¹H NMR (600 MHz, cdcl₃) δ 8.79 (s, 1H), 8.26-8.20 (m, 2H), 8.22-8.19 (m, 2H), 7.72-7.68 (m, 2H), 7.68-7.64 (m, 2H), 7.05 (td, J=7.6, 7.2, 1.8 Hz, 1H), 6.95-6.87 (m, 3H), 3.97 (s, 2H), 3.85 (s, 3H), 3.51 (s, 2H), 3.15 (s, 2H), 2.99 (s, 2H). ¹⁹F NMR (564 MHz, cdcl₃) δ −62.91. ¹³C NMR (151 MHz, cdcl₃) δ 188.28, 167.69, 164.29, 152.35, 145.83, 143.13, 136.89, 132.48 (t, J=16.4 Hz), 129.27, 128.49, 127.58, 125.90-125.66 (m), 123.98 (q, J=272.3 Hz), 121.26, 111.58, 55.58, 51.23, 50.61, 47.92, 42.56. HRMS (ESI-TOF) m/z: [M+H]⁺ Calcd for C₂₇H₂₅F₃N₅O₄S⁺, 572.1574, Found, 572.1562.

### (4-((3-(furan-2-yl)-1H-1,2,4-triazol-1-yl)sulfonyl)phenyl)(4-(2-methoxy-phenyl)-piperazin-1-yl)methanone (RJG-1185)

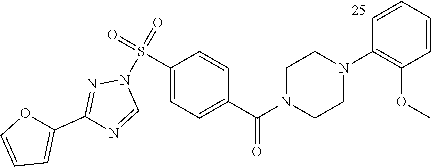 [SMILES: COc1ccccc1N1CCN(C(=O)c2ccc(S(=O)(=O)n3cnc(-c4ccco4)n3)cc2)CC1]

Prepared using Procedure C. White solid (57.0 mg, 43%). ¹H NMR (600 MHz, cdcl₃) δ 8.73 (s, 1H), 8.22-8.16 (m, 2H), 7.67-7.62 (m, 2H), 7.55 (dd, J=1.8, 0.8 Hz, 1H), 7.09 (dd, J=3.4, 0.8 Hz, 1H), 7.04 (ddd, J=8.0, 7.3, 1.8 Hz, 1H), 6.93 (td, J=7.6, 1.3 Hz, 1H), 6.89 (ddd, J=8.1, 5.3, 1.5 Hz, 2H), 6.52 (dd, J=3.4, 1.8 Hz, 1H), 3.96 (s, 2H), 3.86 (s, 3H), 3.51 (s, 2H), 3.14 (s, 2H), 2.99 (s, 2H). ¹³C NMR (151 MHz, cdcl₃) δ 167.73, 158.26, 152.37, 145.69, 144.78, 144.58, 143.06, 136.89, 129.26, 128.48, 123.98, 121.23, 118.64, 112.73, 111.95, 111.52, 55.57, 51.21, 50.61, 48.04, 42.60. FIRMS (ESI-TOF) m/z: [M+H]⁺ Calcd for C₂₄H₂₄N₅O₅S⁺, 494.1493, Found, 494.1492.

### (4-((3-(2-fluorophenyl)-1H-1,2,4-triazol-1-yl)sulfonyl)phenyl)(4-(2-methoxy-phenyl)piperazin-1-yl)methanone (RJG-1187)

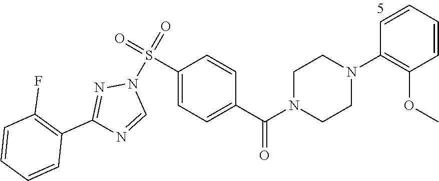 [SMILES: COc1ccccc1N1CCN(C(=O)c2ccc(S(=O)(=O)n3cnc(-c4ccccc4F)n3)cc2)CC1]

Prepared using Procedure C. White solid (25.4 mg, 19%). ¹H NMR (600 MHz, cdcl₃) δ 8.80 (s, 1H), 8.24-8.20 (m, 2H), 8.04 (td, J=7.6, 1.9 Hz, 1H), 7.67-7.63 (m, 2H), 7.46-7.41 (m, 1H), 7.23 (td, J=7.6, 1.1 Hz, 1H), 7.18 (ddd, J=10.7, 8.3, 1.1 Hz, 1H), 7.04 (ddd, J=8.1, 7.3, 1.8 Hz, 1H), 6.93 (td, J=7.6, 1.4 Hz, 1H), 6.89 (ddd, J=8.2, 5.5, 1.5 Hz, 3H), 3.97 (s, 2H), 3.86 (s, 2H), 3.51 (s, 2H), 3.14 (s, 2H), 2.99 (s, 2H). ¹⁹F NMR (564 MHz, cdcl₃) δ 173.28. ¹³C NMR (151 MHz, cdcl₃) δ 188.39, 167.77, 162.08, 160.71 (d, J=257.0 Hz), 152.37, 145.22, 143.03, 140.41, 137.02, 132.32 (d, J=8.6 Hz), 130.64 (d, J=2.2 Hz), 129.28, 128.41, 124.38 (d, J=3.5 Hz), 123.96, 121.23, 118.63, 116.86 (d, J=21.4 Hz), 111.52, 55.56, 51.21, 50.61, 48.05, 42.61. HRMS (ESI-TOF) m/z: [M+H]⁺ Calcd for C₂₆H₂₅FN₅O₄S⁺, 522.1606, Found, 522.1607.

### (4-((3-(3-fluorophenyl)-1H-1,2,4-triazol-1-yl)sulfonyl)phenyl)(4-(2-methoxy-phenyl)piperazin-1-yl)methanone (RJG-1188)

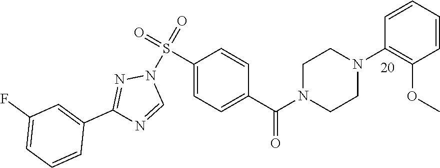 [SMILES: COc1ccccc1N1CCN(C(=O)c2ccc(S(=O)(=O)n3cnc(-c4cccc(F)c4)n3)cc2)CC1]

Prepared using Procedure C. White solid (44.4 mg, 30%). ¹H NMR (600 MHz, cdcl₃) δ 8.75 (s, 1H), 8.21-8.16 (m, 2H), 7.89 (ddd, J=7.8, 1.5, 1.0 Hz, 1H), 7.80 (ddd, J=9.6, 2.7, 1.5 Hz, 1H), 7.66-7.63 (m, 2H), 7.40 (td, J=8.0, 5.7 Hz, 1H), 7.13 (tdd, J=8.4, 2.7, 1.0 Hz, 1H), 7.03 (ddd, J=8.0, 7.3, 1.7 Hz, 1H), 6.91 (td, J=7.6, 1.3 Hz, 1H), 6.87 (ddd, J=8.1, 4.8, 1.5 Hz, 3H), 3.96 (s, 2H), 3.84 (s, 3H), 3.50 (s, 2H), 3.13 (s, 2H), 2.98 (s, 2H). ¹⁹F NMR (564 MHz, cdcl₃) 6-173.91. ¹³C NMR (151 MHz, cdcl₃) δ 167.72, 164.55, 163.03 (d, J 246.3 Hz), 152.36, 145.70, 140.39, 136.97, 131.25 (d, J=7.7 Hz), 130.52 (d, J=8.1 Hz), 129.24, 128.47, 122.98 (d, J=3.0 Hz), 121.23, 118.64, 117.76 (d, J=21.4 Hz), 114.26 (d, J=23.7 Hz), 111.53, 55.57, 51.22, 50.61, 48.06, 42.61. HRMS (ESI-TOF) m/z: [M+H]⁺ Calcd for C₂₆H₂₅FN₅O₄S⁺, 522.1606, Found, 522.1608.

### (4-(2-methoxyphenyl)piperazin-1-yl)(4-((3-(pyridin-3-yl)-1H-1,2,4-triazol-1-yl)sulfonyl)phenyl)methanone (RJG-1189)

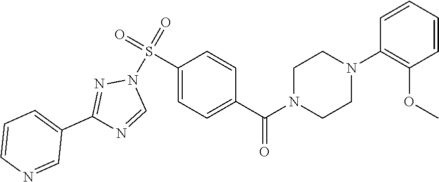 [SMILES: COc1ccccc1N1CCN(C(=O)c2ccc(S(=O)(=O)n3cnc(-c4cccnc4)n3)cc2)CC1]

Prepared using Procedure C. White solid (52.3 mg, 52%). ¹H NMR (600 MHz, cdcl₃) δ 9.32 (s, 1H), 8.78 (s, 1H), 8.68 (d, J=4.8 Hz, 1H), 8.36 (dt, J=8.0, 1.9 Hz, 1H), 8.22-8.16 (m, 2H), 7.71-7.60 (m, 2H), 7.38 (dd, J=8.0, 4.8 Hz, 1H), 7.03 (td, J=7.7, 1.7 Hz, 1H), 6.94-6.84 (m, 3H), 3.95 (s, 2H), 3.84 (s, 3H), 3.50 (s, 2H), 3.13 (s, 2H), 2.98 (s, 2H). ¹³C NMR (151 MHz, cdcl₃) δ 167.68, 163.33, 152.36, 151.50, 148.50, 145.83, 143.19, 140.35, 136.84, 134.60, 129.29, 128.50, 124.00, 123.70, 121.23, 118.64, 111.52, 55.57, 51.22, 50.59, 48.06, 42.61. HRMS (ESI-TOF) m/z: [M+H]⁺ Calcd for C₂₅H₂₅N₆O₄S⁺, 505.1653, Found, 505.1647.

### (4-(2-methoxyphenyl)piperazin-1-yl)(4-((3-(4-(trifluoromethoxy)phenyl)-1H-1,2,4-triazol-1-yl)sulfonyl)phenyl)methanone (RJG-1228)

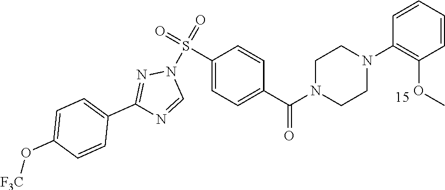 [SMILES: COc1ccccc1N1CCN(C(=O)c2ccc(S(=O)(=O)n3cnc(-c4ccc(OC(F)(F)F)cc4)n3)cc2)CC1]

Prepared using Procedure B. White solid (161 mg, 81%). ¹H NMR (497 MHz, cdcl₃) δ 8.76 (s, 1H), 8.22-8.18 (m, 2H), 8.18-8.13 (m, 2H), 7.68-7.63 (m, 2H), 7.30-7.27 (m, 2H), 7.07-7.01 (m, 1H), 6.95-6.86 (m, 3H), 3.97 (s, 2H), 3.86 (s, 3H), 3.51 (s, 2H), 3.14 (s, 2H), 2.99 (s, 2H). ¹⁹F NMR (564 MHz, cdcl₃) δ −57.75. ¹³C NMR (151 MHz, cdcl₃) δ 188.52, 167.73, 164.44, 152.38, 151.07, 145.77, 143.10, 140.38, 137.01, 129.23, 128.97, 128.47, 127.78, 124.02, 121.25, 121.12, 118.64, 111.54, 55.58, 51.24, 50.61, 48.08, 42.63. HRMS (ESI-TOF) m/z: [M+H]⁺ Calcd for C₂₇H₂₅F₃N₅O₅S⁺, 588.1523, Found, 588.1537.

### (4-(4-iodophenyl)piperazin-1-yl)(4-((3-phenyl-1H-1,2,4-triazol-1-yl)sulfonyl)-phenyl)methanone (RJG-1257)

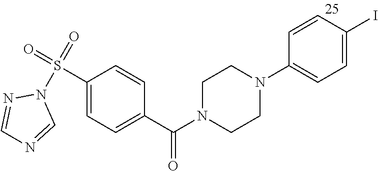 [SMILES: O=C(c1ccc(S(=O)(=O)n2cncn2)cc1)N1CCN(c2ccc(I)cc2)CC1]

Prepared using Procedure A. White solid (93.4 mg, 74%). ¹H NMR (497 MHz, cdcl₃) δ 8.76 (s, 1H), 8.19-8.14 (m, 2H), 8.06 (s, 1H), 7.68-7.61 (m, 2H), 7.57-7.51 (m, 2H), 6.70-6.65 (m, 2H), 3.93 (s, 2H), 3.48 (s, 2H), 3.24 (s, 2H), 3.08 (s, 2H). ¹³C NMR (151 MHz, cdcl₃) δ 167.74, 154.71, 150.41, 144.91, 142.73, 138.22, 137.07, 129.32, 128.47, 118.95, 49.76, 49.21, 47.42, 41.85. HRMS (ESI-TOF) m/z: [M+H]⁺ Calcd for C₁₉H₁₉IN₅O₃S⁺, 524.0248, Found, 524.0226.

### (4-((1H-1,2,4-triazol-1-yl)sulfonyl)phenyl)(4-(pyridin-2-yl)piperazin-1-yl)methanone (RJG-1285)

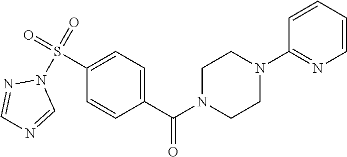 [SMILES: O=C(c1ccc(S(=O)(=O)n2cncn2)cc1)N1CCN(c2ccccn2)CC1]

Prepared using Procedure A. White solid (40.6 mg, 41%). ¹H NMR (600 MHz, cdcl₃) δ 8.76 (s, 1H), 8.20 (ddd, J=4.9, 2.0, 0.8 Hz, 1H), 8.19-8.15 (m, 2H), 8.06 (s, 1H), 7.69-7.62 (m, 2H), 7.52 (ddd, J=8.5, 7.1, 2.0 Hz, 1H), 6.70 (ddd, J=7.2, 4.9, 0.8 Hz, 1H), 6.66 (dt, J=8.5, 0.9 Hz, 1H), 3.90 (s, 2H), 3.64 (s, 2H), 3.54 (s, 2H), 3.45 (s, 2H). ¹³C NMR (151 MHz, cdcl₃) δ 167.88, 159.03, 154.71, 148.24, 144.90, 142.90, 137.94, 137.01, 129.32, 128.48, 114.53, 107.56, 47.40, 45.66, 45.39, 42.08. HRMS (ESI-TOF) m/z: [M+H]⁺ Calcd for C₁₈H₁₉N₆O₃S⁺, 399.1234, Found, 399.1239.

### (4-(2-methoxyphenyl)piperazin-1-yl)(4-((3-(pyridin-4-yl)-1H-1,2,4-triazol-1-yl)sulfonyl)phenyl)methanone (RJG-1291)

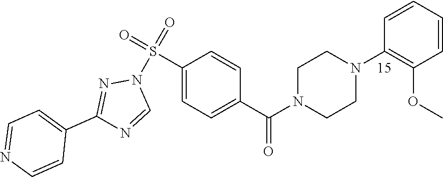 [SMILES: COc1ccccc1N1CCN(C(=O)c2ccc(S(=O)(=O)n3cnc(-c4ccncc4)n3)cc2)CC1]

Prepared using Procedure A. White solid (64.6 mg, 38%). ¹H NMR (497 MHz, cdcl₃) δ 8.81 (s, 1H), 8.78-8.71 (m, 2H), 8.24-8.19 (m, 2H), 8.03-7.96 (m, 2H), 7.69-7.64 (m, 2H), 7.05 (ddd, J=8.1, 7.3, 1.8 Hz, 1H), 6.96-6.86 (m, 3H), 3.97 (s, 2H), 3.86 (s, 3H), 3.51 (s, 2H), 3.14 (s, 2H), 2.99 (s, 2H). ¹³C NMR (151 MHz, cdcl₃) δ 167.78, 163.33, 150.45, 145.94, 143.42, 140.46, 136.73, 129.35, 128.56, 124.04, 121.25, 121.21, 118.64, 111.55, 55.58, 51.55, 50.95, 48.23, 42.67. HRMS (ESI-TOF) m/z: [M+H]⁺ Calcd for C₂₅H₂₅N₆O₄S⁺, 505.1653, Found, 505.1663.

### (4-((3-([1,1′-biphenyl]-4-yl)-1H-1,2,4-triazol-1-yl)sulfonyl)phenyl)(4-(2-methoxyphenyl)piperazin-1-yl)methanone (RJG-2011)

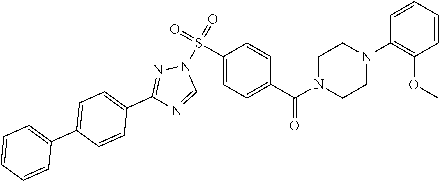 [SMILES: COc1ccccc1N1CCN(C(=O)c2ccc(S(=O)(=O)n3cnc(-c4ccc(-c5ccccc5)cc4)n3)cc2)CC1]

Prepared using Procedure B. Off-white solid (97.4 mg, 45%). ¹H NMR (497 MHz, cdcl₃) δ 8.78 (s, 1H), 8.24-8.17 (m, 4H), 7.65 (s, OH), 7.65-7.59 (m, 2H), 7.46 (t, J=7.7 Hz, 2H), 7.39-7.34 (m, 1H), 7.04 (td, J=7.7, 1.8 Hz, 1H), 6.94-6.86 (m, 3H), 3.97 (s, 2H), 3.85 (s, 3H), 3.51 (s, 2H), 3.14 (s, 2H), 2.99 (s, 2H). ¹³C NMR (151 MHz, cdcl₃) δ 167.79, 165.39, 152.35, 145.69, 143.58, 142.84, 140.36, 137.23, 129.20, 129.03, 128.45, 127.99, 127.73, 127.53, 127.25, 121.31, 111.62, 55.61, 51.29, 50.70, 47.89, 42.46. HRMS (ESI-TOF) m/z: [M+H]⁺ Calcd for C₃₂H₃₀N₅O₄S⁺, 580.2013, Found, 580.2021.

### (3-((1H-1,2,4-triazol-1-yl)sulfonyl)phenyl)(4-(2-methoxyphenyl)piperazin-1-yl)methanone (RJG-2036)

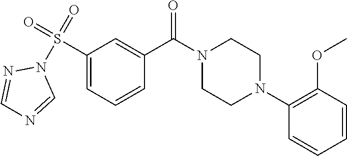 [SMILES: COc1ccccc1N1CCN(C(=O)c2cccc(S(=O)(=O)n3cncn3)c2)CC1]

Prepared using Procedure B. White solid (415 mg, 82%). ¹H NMR (497 MHz, cdcl₃) δ 8.75 (s, 1H), 8.16 (ddd, J=6.1, 1.9, 1.2 Hz, 2H), 8.04 (s, 1H), 7.82 (dt, J=7.7, 1.3 Hz, 1H), 7.74-7.67 (m, 1H), 7.09 (s, 1H), 6.99-6.89 (m, 2H), 4.03 (d, J=17.4 Hz, 2H), 3.89 (s, 3H), 3.58 (s, 2H), 3.20 (s, 2H), 3.08 (s, 2H). ¹³C NMR (201 MHz, cdcl₃) δ 167.48, 154.71, 152.40, 144.90, 136.51, 134.41, 130.43, 129.91, 127.55, 121.43, 111.81, 55.71, 51.31, 50.79, 48.04, 42.26. HRMS (ESI-TOF) m/z: [M+H]⁺ Calcd for C₂₀H₂₂N₅O₄S⁺, 428.1387, Found, 428.1387.

### 4-((3-cyclopropyl-1H-1,2,4-triazol-1-yl)sulfonyl)-N-(prop-2-yn-1-yl)benzamide (RJG-2043)

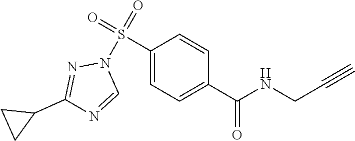 [SMILES: C#CCNC(=O)c1ccc(S(=O)(=O)n2cnc(C3CC3)n2)cc1]

Prepared using Procedure B. White solid (403 mg, 61%). ¹H NMR (497 MHz, cdcl₃) δ 8.55 (s, 1H), 8.15-8.11 (m, 2H), 8.00-7.95 (m, 2H), *7.94 (d, J=8.4 Hz, 2H), *7.85 (d, J=8.4 Hz, 2H), *7.73 (s, 1H), 6.39 (s, 1H), 4.29-4.25 (m, 2H), *2.84-2.77 (m, 1H), 2.31 (dd, J=2.9, 2.1 Hz, 1H), 2.02 (ddt, J=10.3, 7.9, 3.9 Hz, 1H), *1.25-1.19 (m, 2H), *1.19-1.15 (m, 2H), 1.01-0.95 (m, 4H). ¹³C NMR (201 MHz, cdcl₃) δ 170.30, 165.07, *162.66, *152.12, 145.24, 140.03, 139.05, 129.08, *128.87, 128.46, *128.42, *128.17, *127.95, 78.77, 72.69, 30.25, *10.91, 9.17 , 8.56. HRMS (ESI-TOF) m/z: [M+H]⁺ Calcd for C₁₅H₁₅N₄O₃S⁺, 331.0859, Found, 331.0823.

* indicates rotamer peaks

### 4-((3-cyclobutyl-1H-1,2,4-triazol-1-yl)sulfonyl)-N-(prop-2-yn-1-yl)benzamide (RJS-1027)

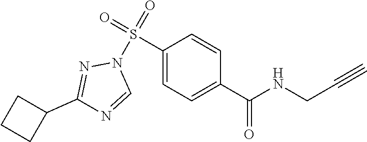 [SMILES: C#CCNC(=O)c1ccc(S(=O)(=O)n2cnc(C3CCC3)n2)cc1]

Prepared using Procedure B. White solid (312 mg, 61%). ¹H NMR (497 MHz, cdcl₃) δ 8.64 (s, 1H), 8.15-8.11 (m, 2H), *8.10-8.07 (m, 2H), 7.98-7.94 (m, 3H), *7.84 (s, 1H), 6.56 (t, J=5.4 Hz, 1H), 4.24 (dd, J=5.2, 2.5 Hz, 3H), *419-4.13 (m, 1H), 3.64-3.55 (m, 1H), 2.51-2.40 (m, 1H), 2.38-2.23 (m, 5H), *2.18-2.05 (m, 2H), 2.07-1.95 (m, 1H), *1.98-1.86 (m, 2H). ¹³C NMR (201 MHz, cdcl₃) δ 171.25, 165.09 (d, J=2.9 Hz), *163.04, 152.10, 145.40, 140.05, *13991, *13971, 138.99, 129.08, *12893, *12862, 128.48, *12839, *12754, 78.78, 72.62, 33.48, *32.42, 30.21, *27.88, 27.63, 18.79, *18.50. HRMS (ESI-TOF) m/z: [M+H]⁺ Calcd for C₁₆H₁₇N₄O₃S⁺, 345.1016, Found, 345.1043.

* indicates rotamer peaks

### 4-((3-cyclopentyl-1H-1,2,4-triazol-1-yl)sulfonyl)-N-(prop-2-yn-1-yl)benzamide (RJG-2044)

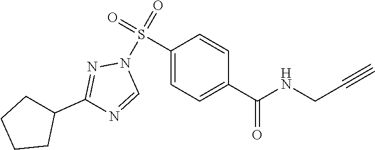 [SMILES: C#CCNC(=O)c1ccc(S(=O)(=O)n2cnc(C3CCCC3)n2)cc1]

Prepared using Procedure B. White solid (315 mg, 49%). ¹H NMR (600 MHz, cdcl₃) δ 8.62 (s, 1H), 8.16-8.11 (m, 2H), 8.00-7.94 (m, 2H), *7.80 (s, 1H), 6.41 (s, 1H), 4.26 (dd, J=5.2, 2.6 Hz, 2H), *3.86 (p, J=7.9 Hz, 1H), 3.16 (p, J=7.8 Hz, 1H), 2.30 (t, J=2.6 Hz, 1H), *2.19-2.10 (m, 2H), 2.03-1.94 (m, 2H), *1.93-1.81 (m, 2H), 1.81-1.68 (m, 2H), 1.62 (dq, J=9.3, 3.6, 3.0 Hz, 2H). ¹³C NMR (201 MHz, cdcl₃) δ 172.34, 165.09, *164.68, *151.93, 145.35, 140.01, 139.90, 139.08, 129.08, *129.01, *128.61, 128.45, *128.38, *127.56, 78.78, 72.66, 38.80, *37.31, *32.89, 32.12, *30.28, 30.23, *25.89, 25.74. HRMS (ESI-TOF) m/z: [M+H]⁺ Calcd for C₁₇H₁₉N₄O₃S⁺, 359.1172, Found, 359.1171.

* indicates rotamer peaks

### 4-((3-cyclohexyl-1H-1,2,4-triazol-1-yl)sulfonyl)-N-(prop-2-yn-1-yl)benzamide (RJG-2045)

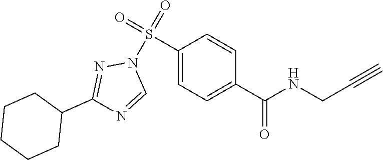 [SMILES: C#CCNC(=O)c1ccc(S(=O)(=O)n2cnc(C3CCCCC3)n2)cc1]

Prepared using Procedure B. White solid (320 mg, 45%). ¹H NMR (600 MHz, cdcl₃) δ 8.62 (s, 1H), 8.15-8.11 (m, 2H), 7.99-7.95 (m, 2H), *7.81 (s, 1H), 6.45 (s, 1H), 4.25 (dd, J=5.2, 2.6 Hz, 2H), *3.48 (tt, J=11.8, 3.5 Hz, 1H), 2.72 (tt, J=11.6, 3.6 Hz, 1H), 2.30 (t, J=2.6 Hz, 1H), 1.99-1.90 (m, 2H), *1.87 (ddd, J=13.3, 5.0, 2.0 Hz, 2H), 1.76 (dq, J=12.7, 3.5 Hz, 2H), 1.68 (dddd, J=12.6, 5.0, 3.2, 1.4 Hz, 1H), *1.62 (qd, J=12.8, 3.4 Hz, 2H), 1.49 (qd, J=12.3, 3.5 Hz, 2H), *1.45-1.41 (m, 2H), 1.32 (qt, J=12.4, 3.3 Hz, 2H), 1.27-1.19 (m, 1H). ¹³C NMR (201 MHz, cdcl₃) δ 172.43, 165.09, *164.56, 152.07, 145.16, 139.99, 139.11, 129.05, *129.02, 128.42, *128.40, 78.79, 72.69, 37.74, *36.80, 32.07, 31.25, 30.24, 26.02, 25.88, *25.70. HRMS (ESI-TOF) m/z: [M+H]⁺ Calcd for C₁₈H₂₁N₄O₃S⁺, 373.1329, Found, 373.1329.

* indicates rotamer peaks

### (4-((3-cyclopropyl-1H-1,2,4-triazol-1-yl)sulfonyl)phenyl)(4-(2-methoxy-phenyl)piperazin-1-yl)methanone (RJG-2048)

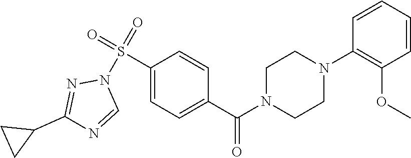 [SMILES: COc1ccccc1N1CCN(C(=O)c2ccc(S(=O)(=O)n3cnc(C4CC4)n3)cc2)CC1]

Prepared using Procedure B. Off-white solid (430 mg, 60%). ¹H NMR (600 MHz, cdcl₃) δ 8.54 (s, 1H), 8.12 (d, J=8.3 Hz, 2H), 7.65-7.62 (m, 2H), 7.05 (td, J=7.2, 5.8, 2.5 Hz, 1H), 6.97-6.88 (m, 3H), 3.98 (s, 2H), 3.86 (s, 3H), 3.53 (s, 2H), 3.15 (s, 2H), 3.01 (s, 2H), 2.07-2.00 (m, 1H), 0.99 (d, J=6.6 Hz, 4H). ¹³C NMR (201 MHz, cdcl₃) δ 170.14, 167.86, *162.53, 152.34, * 152.02, 145.17, 142.70, *140.25, 137.24, 129.05, *128.85, 128.32, *128.27, *128.18, *127.80, 124.05, 121.22, 118.68, 111.52, 55.56, 51.20, 50.60, 48.00, 42.55, 9.15, 8.52. HRMS (ESI-TOF) m/z: [M+H]⁺ Calcd for C₂₃H₂₆N₅O₄S⁺, 468.1700, Found, 468.1707.

* indicates rotamer peaks

### (4-((3-cyclobutyl-1H-1,2,4-triazol-1-yl)sulfonyl)phenyl)(4-(2-methoxy-phenyl)piperazin-1-yl)methanone (RJG-2049)

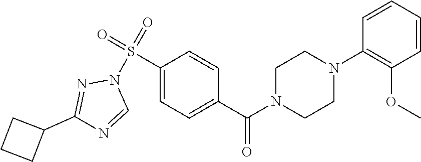 [SMILES: COc1ccccc1N1CCN(C(=O)c2ccc(S(=O)(=O)n3cnc(C4CCC4)n3)cc2)CC1]

Prepared using Procedure B. White solid (405 mg, 54%). ¹H NMR (600 MHz, cdcl₃) δ 8.62 (s, 1H), 8.13-8.10 (m, 1H), *8.10-8.07 (m, 1H), *7.83 (s, 1H), 7.64-7.58 (m, 2H), 7.06-7.00 (m, 1H), 6.94-6.84 (m, 3H), 3.96 (d, J=6.7 Hz, 2H), *4.12 (m, 1H), 3.85 (s, 3H), 3.61 (p, J=8.6 Hz, 1H), 3.50 (s, 2H), 3.13 (s, 2H), 2.98 (s, 2H), 2.49-2.42 (m, 1H), 2.38-2.27 (m, 3H), *2.14-2.06 (m, 1H), 2.06-1.99 (m, 1H), 1.98-1.89 (m, 1H). ¹³C NMR (201 MHz, cdcl₃) δ 171.12, 167.81, *162.90, 152.33,*152.03, 145.32, 142.73, *142.59 *140.33, *137.93, 137.21, *129.08, 128.91, 128.30, *128.23, *128.16, *127.79, 123.97, 121.20, 118.62, 111.50, *60.48, 55.54, 51.17, 50.58, 47.99, 42.53, 33.47, *32.42, *27.86, 27.62, 18.77, *1848 HRMS (ESI-TOF) m/z: [M+H]⁺ Calcd for C₂₄H₂₈N₅O₄S⁺, 482.1857, Found, 482.1867.

* indicates rotamer peaks

### (4-((3-cyclopentyl-1H-1,2,4-triazol-1-yl)sulfonyl)phenyl)(4-(2-methoxyphenyl)-piperazin-1-yl)methanone (RJG-2050)

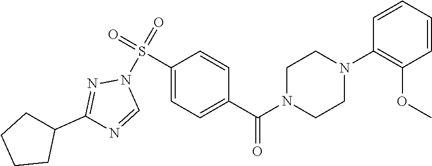 [SMILES: COc1ccccc1N1CCN(C(=O)c2ccc(S(=O)(=O)n3cnc(C4CCCC4)n3)cc2)CC1]

Prepared using Procedure B. White solid (337 mg, 49%). ¹H NMR (600 MHz, cdcl₃) δ 8.62 (s, 1H), 8.15-8.10 (m, 2H), *7.81 (s, 1H), 7.66-7.60 (m, 2H), 7.05 (t, J=8.3 Hz, 1H), 6.96-6.84 (m, 3H), 3.97 (s, 2H), 3.86 (s, 3H), 3.52 (s, 2H), 3.22-3.12 (m, 3H), 3.00 (s, 2H), *2.18-2.11 (m, 2H), 2.05-1.96 (m, 2H), *1.94-1.88 (m, 2H), *1.88-1.82 (m, 2H), 1.84-1.68 (m, 2H), 1.69-1.58 (m, 1H). ¹³C NMR (201 MHz, cdcl₃) δ 172.25, 167.93, 152.40, 145.35, 142.77, 139.88, 137.34, 129.12, 128.35, 124.08, 121.29, 118.73, 111.60, 55.61, 51.25, 50.66, 48.04, 42.58, 38.81, 32.15, 25.76. HRMS (ESI-TOF) m/z: [M+H]⁺ Calcd for C₂₅H₃₀N₅O₄S⁺, 496.2013, Found, 496.2026.

* indicates rotamer peaks

### (4-((3-cyclohexyl-1H-1,2,4-triazol-1-yl)sulfonyl)phenyl)(4-(2-methoxyphenyl)-piperazin-1-yl)methanone (RJG-2051)

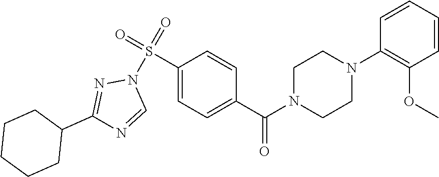 [SMILES: COc1ccccc1N1CCN(C(=O)c2ccc(S(=O)(=O)n3cnc(C4CCCCC4)n3)cc2)CC1]

Prepared using Procedure B. White solid (180 mg, 33%). ¹H NMR (600 MHz, cdcl₃) δ 8.61 (s, 1H), 8.16-8.11 (m, 2H), *7.85 (d, J=8.0 Hz, 0-2H), *7.83 (s, 1H), 7.65-7.60 (m, 2H), 7.05 (d, J=8.2 Hz, 1H), 6.91 (dd, J=21.9, 7.6 Hz, 3H), 3.98 (s, 2H), 3.87 (s, 3H), 3.53 (s, 2H), 3.16 (s, 2H), 3.01 (s, 2H), 2.78-2.72 (m, 1H), 2.01-1.91 (m, 2H), 1.88 (d, J=13.3 Hz, 2H), 1.78 (dt, J=13.1, 3.5 Hz, 2H), *1.69 (d, J=12.6 Hz, 2H), *1.63 (dd, J=12.2, 3.4 Hz, 2H), 1.62-1.40 (m, 4H), 1.39-1.29 (m, 2H), 1.29-1.21 (m, 1H). ¹³C NMR (201 MHz, cdcl₃) δ 172.34, 167.89, *164.49, 152.38, 152.04, 145.11, *137.38, 129.09, *129.06, 128.32, *128.27, *128.23, *127.84, 121.29, 111.60, 55.62, 51.26, 50.68, 47.97, 37.75, 36.81, 32.11, 31.26, 26.05, 25.90, *25.72. HRMS (ESI-TOF) m/z: [M+H]⁺ Calcd for C₂₆H₃₂N₅O₄S⁺, 510.2170, Found, 510.2175.

* indicates rotamer peaks

### (4-((3-(4-methoxyphenyl)-1H-1,2,4-triazol-1-yl)sulfonyl)phenyl)(4-(2-methoxy-phenyl)piperazin-1-yl)methanone (RJG-2056)

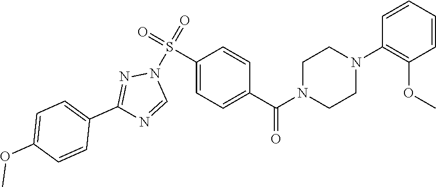 [SMILES: COc1ccc(-c2ncn(S(=O)(=O)c3ccc(C(=O)N4CCN(c5ccccc5OC)CC4)cc3)n2)cc1]

Prepared using Procedure B. Light yellow solid (437 mg, 64%). ¹H NMR (600 MHz, cdcl₃) δ 8.72 (s, 1H), 8.20-8.16 (m, 2H), 8.07-8.02 (m, 2H), 7.66-7.62 (m, 2H), 7.04 (td, J=7.7, 1.8 Hz, 1H), 6.97-6.93 (m, 2H), 6.92 (dd, J=7.4, 1.3 Hz, 1H), 6.88 (ddd, J=8.2, 5.0, 1.5 Hz, 2H), 3.97 (s, 2H), 3.85 (s, 3H), 3.85 (s, 3H), 3.51 (s, 2H), 3.14 (s, 2H), 2.99 (s, 2H). ¹³C NMR (201 MHz, cdcl₃) δ 167.76, 165.41, 161.70, 152.31, 145.55, 142.81, 140.37, 137.21, 129.08, 128.82, 128.42, 128.35, 127.56, 123.91, 121.68, 121.18, 118.58, 114.19, 111.47, 55.52, 55.47, 51.17, 50.56, 48.01, 42.56. HRMS (ESI-TOF) m/z: [M+H]⁺ Calcd for C₂₇H₂₈N₅O₅S⁺, 534.1806, Found, 534.1802.

### (4-((1H-1,2,4-triazol-1-yl)sulfonyl)phenyl)(4-((2-methoxyphenyl)amino)-piperidin-1-yl)methanone (RJG-2058)

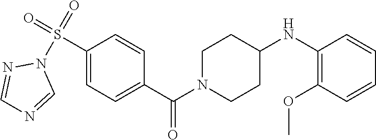 [SMILES: COc1ccccc1NC1CCN(C(=O)c2ccc(S(=O)(=O)n3cncn3)cc2)CC1]

Prepared using Procedure B. White solid (14 mg, 45%). ¹H NMR (600 MHz, cdcl₃) δ 8.76 (s, 1H), 8.17-8.11 (m, 2H), 8.05 (s, 1H), 7.65-7.59 (m, 2H), 6.85 (td, J=7.7, 1.4 Hz, 1H), 6.79 (dd, J=8.0, 1.4 Hz, 1H), 6.69 (t, J=7.7 Hz, 1H), 6.62 (d, J=7.9 Hz, 1H), 4.52 (s, OH), 3.84 (s, 3H), 3.62-3.54 (m, 2H), 3.21-3.12 (m, 3H), 2.21 (s, 1H), 2.09-2.02 (m, 1H), 1.56 (s, 1H), 1.41 (t, J=7.2 Hz, OH), 1.30-1.23 (m, 1H). ¹³C NMR (201 MHz, cdcl₃) δ 167.72, 154.67, 147.08, 144.88, 143.39, 136.75, 136.30, 129.27, 128.19, 121.36, 117.22, 110.57, 109.95, 55.56, 49.61, 46.49, 41.24, 32.99, 32.09, 29.84, 28.61. HRMS (ESI-TOF) m/z: [M+H]⁺ Calcd for C₂₁H₂₄N₅O₅S⁺, 442.1544, Found, 442.1543.

### Example 9

### HPLC Purity of PTGR2 Inhibitors

Samples were injected on a Shimadzu 1100 Series spectrometer (Shimadzu, Kyoto, Japan) with UV detection at 254 nm using a reverse-phase C₁₋₈ chromatograpy column sold under the tradename KINETEX® (Phenomenex Inc., Torrance, California, United States of America) (50×4.6 mm, 2.6 μm, 100 Å) using a 10-minute 15-85% gradient of solvent B in solvent A. Solvent A: HPLC-grade water with 0.1% acetic acid; Solvent B: HPLC-grade acetonitrile with 0.1% acetic acid.

Purity was calculated from the HPLC spectra. Results are described in Table 5, below.

TABLE 5
HPLC Purity of PTGR2 Inhibitors.

| Compound | HPLC Purity | Retention Time |
|---|---|---|
| HHS-0101 | 97.7% | 3.007 min. |
| HHS-0201 | 99.4% | 3.630 min. |
| HHS-0301 | 99.2% | 4.407 min. |
| HHS-0401 | 99.1% | 4.191 min. |
| HHS-0701 | 98.9% | 4.943 min. |
| RJG-1101 | 99.1% | 5.859 min. |
| RJG-1103 | 97.7% | 4.848 min. |
| RJG-1105 | 96.9% | 4.589 min. |
| RJG-1112 | 98.9% | 6.490 min. |
| RJG-1114 | 97.8% | 5.998 min. |
| RJG-1115 | 98.3% | 6.525 min. |
| RJG-1185 | 98.6% | 5.211 min. |
| RJG-1187 | 98.7% | 5.764 min. |
| RJG-1188 | 98.8% | 6.058 min. |
| RJG-1189 | 97.9% | 4.831 min. |
| RJG-1228 | 95.1% | 6.628 min. |
| RJG-1257 | 97.8% | 5.496 min. |
| RJG-1285 | 99.6% | 2.541 min. |
| RJG-1291 | 99.6% | 4.788 min. |
| RJG-2011 | 96.5% | 6.810 min. |
| RJG-2036 | 96.6% | 4.554 min. |
| RJG-2043 | 95.5% | 4.001 min. |
| RJG-2044 | 95.1% | 4.757 min. |
| RJG-2045 | 99.2% | 5.151 min. |
| RJG-2048 | 95.2% | 5.194 min. |
| RJG-2049 | 98.1% | 5.258 min. |
| RJG-2050 | 98.3% | 5.816 min. |
| RJG-2051 | 95.0% | 6.179 min. |
| RJS-1027 | 98.6% | 4.399 min. |
| RJG-2056 | 97.8% | 5.825 min. |
| RJG-2058 | 95.9% | 4.667 min. |

### Example 10

In Vitro and In Situ Inhibition of Probe Labeling with Sulfonyl-Triazole PTGR2 Inhibitors

### Cell Culture:

HEK293T cells were cultured at 37° C. with 5% CO₂ in DMEM with 10% fetal bovine serum and 1% L-glutamine in 10 cm² tissue culture dishes. Cells were grown to 80% confluency for experimental use or to passage.

### Cell Treatments

HEK293T cells were grown to 30-40% confluency and transiently transfected with wild type PTGR2 or Y100F PTGR2 mutant plasmid or DMSO by dissolving 20 μL of PEI (1 mg/mL) and 2.6 μg of plasmid in 0.6 mL of serum-free DMEM media. Transfected HEK293T cells were incubated at 37° C. with 5% CO₂ and were harvested for in vitro experiments. For in situ experiments, cells were treated with either DMSO or inhibitors at a final concentration of 1 μM, or the indicated concentration, of inhibitors from a 1,000× stock in serum-free media. Cells were subsequently incubated at 37° C. with 5% CO₂ for 2 hours, followed by treatment with DMSO or probe at a final concentration of 1 μM from a 1,000× stock in serum-free media. Cells were harvested and pelleted at 400×g for 5 min and the supernatant was decanted. Cells were re-suspended in cold PBS and centrifuged at 400×g for 5 min and the supernatant was decanted once more. The PBS wash was repeated for a second time before cells were snap frozen and stored at −80° C. for future experiments. Dose-response assays were performed to optimize treatment conditions for RJG-2036 in a similar manner.

### Gel-Based Chemical Proteomics Assay:

Cell pellets were lysed in PBS buffer (EDTA free) by sonicating 3 times for 1 second×20% amplitude. The lysate was fractionated by centrifuging at 100,000×g for 45 min at 4° C., separating membrane and soluble fractions. Only the soluble fraction was used for in vitro and in situ experiments. Protein concentrations were measured using the Bio-Rad DC protein assay, and fractions were diluted to a concentration of 2 mg/mL in PBS. For live cell treated samples, 50 μL aliquots of proteome were used for gel experiments. For in vitro experiments, 48 μL aliquots of cell lysate were used for each inhibitor before adding 1 μL of 50× inhibitor stock (1 μM final) and incubated for 30 minutes at 37° C., then 1 μL of 50×probe stock (1 μM final) was added and the lysate incubated for 30 minutes at 37° C. Addition of the rhodamine fluorescent tag was accomplished by adding CuAAC reagents in the following manner: 1 μl of 1.25 mM stock of rhodamine-azide in DMSO (25 μM final), 1 μl of freshly prepared 50 mM TCEP stock in water (1 mM final), 3 μl of a 1.7 mM TCEP stock in 4:1 t-butanol/DMSO (100 μM final), and 1 μl of a 50 mM CuSO₄ stock (1 mM final concentration). Samples were immediately and gently flicked, and the reaction proceeded for 1 hr at room temperature. Reactions were quenched with 17 μL of 4× SDSPAGE loading buffer and β-mercaptoethanol. 30 μL of each sample were separated by SDSPAGE and analyzed by in-gel fluorescence scanning for the rhodamine azide tag. Coomassie staining or Western blot, e.g., using anti-Flag and GAPDH primary antibodies produced in rabbit followed by secondary antibody treatment with a conjugated fluorescent tag sold under the tradename DYLIGHT 650 (Pierce Biotechnology, Ltd., Rockford, Illinois, United States of America), were used to control for equivalent protein loading and presence of recombinant protein across lanes.

### Results:

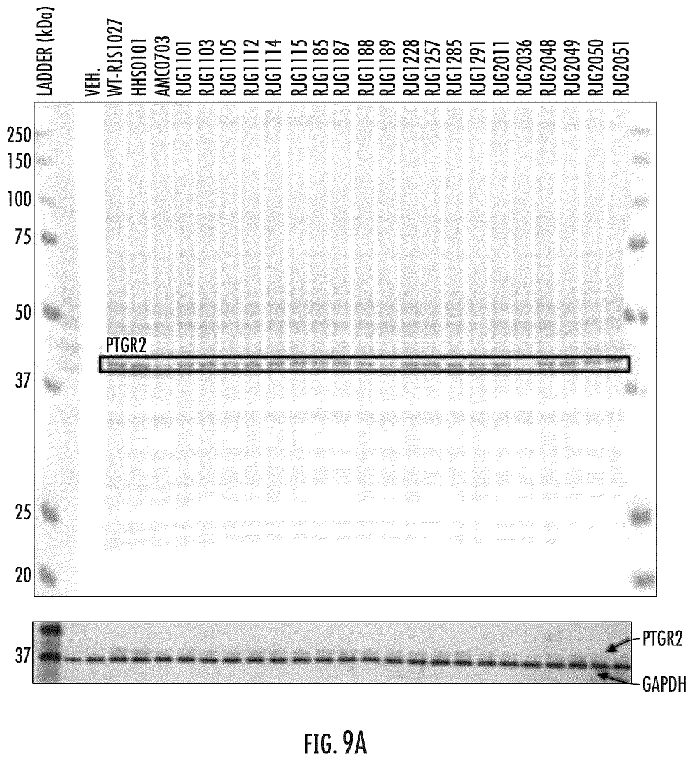

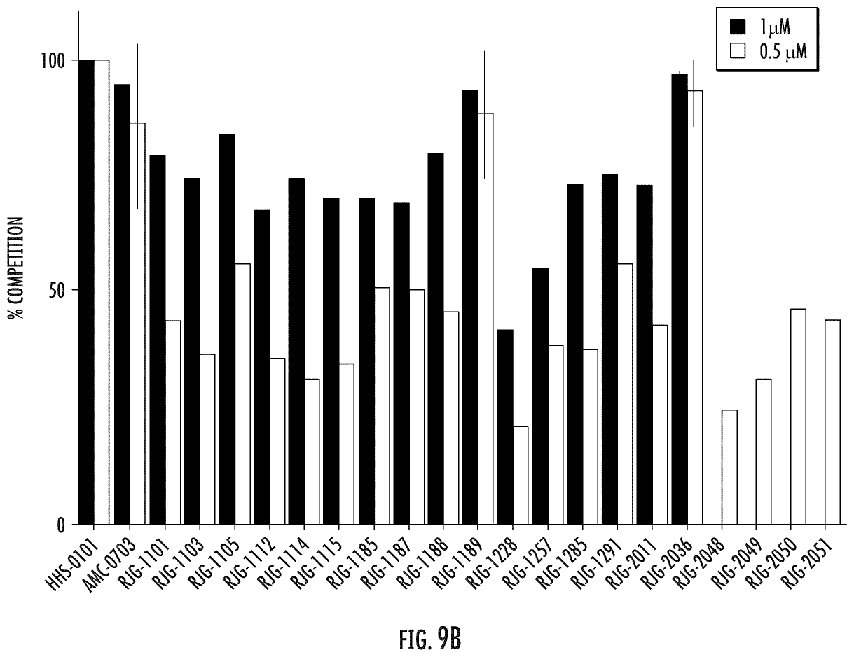

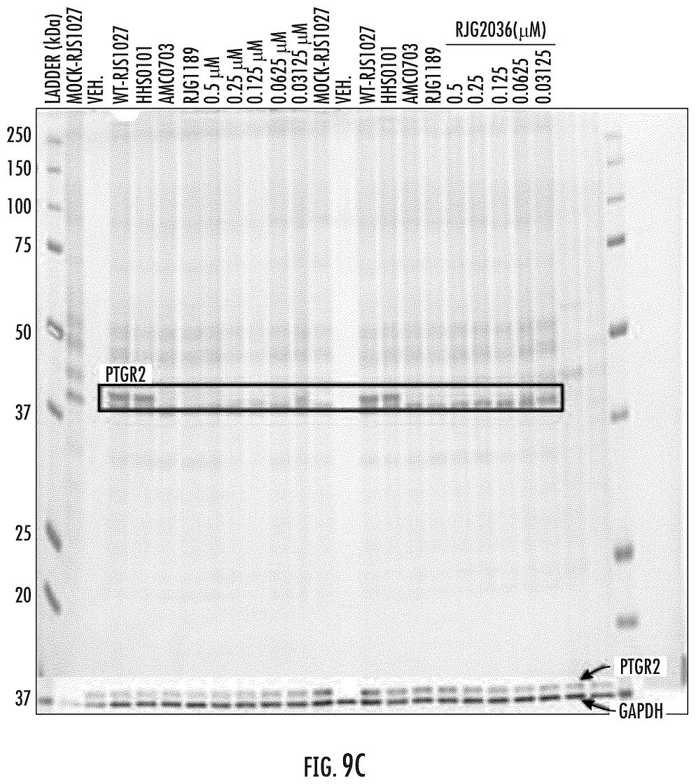

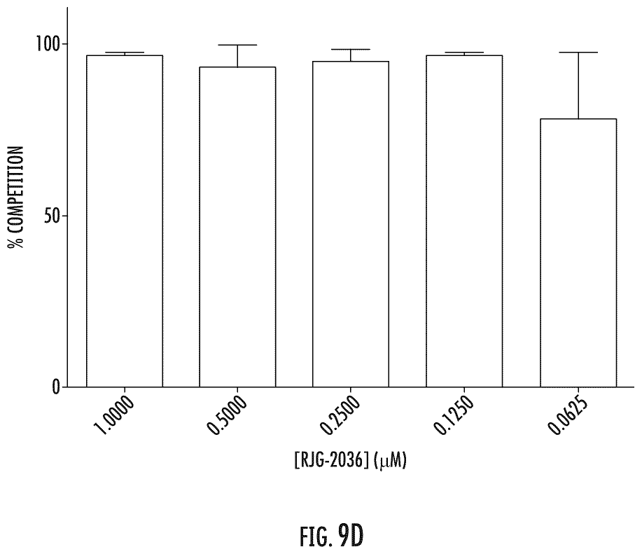

FIG. 9A is an image of an in vitro gel-based chemical proteomics assay using RJS-1027 (1 μM) as a probe to measure competition of binding PTGR2 with an inhibitor (0.5 μM). Soluble proteome harvested from HEK293T cells overexpressing PTGR2 was treated with DMSO or inhibitor for 30 minutes at 37° C. followed by treatment with the probe for 30 minutes at 37° C. Anti-flag and GAPDH western blots are shown below the long gel and were performed separately. Quantitative comparison of inhibitor competition with probe labeling normalized to the negative control HHS-0101 is shown in FIG. 9B. FIG. 9C shows an image of an in vitro gel-based chemical proteomics assay using RJS-1027 (1 μM) as a probe to measure competition of binding PTGR2 with select potent inhibitors, e.g., AMC-0703 (0.5 μM) and RJG-1189 (0.5 μM), and a dose response with RJG-2036. Quantitative comparison of RJG-2036 competition with probe labeling normalized to the negative control HHS-0101 is shown in FIG. 9D.

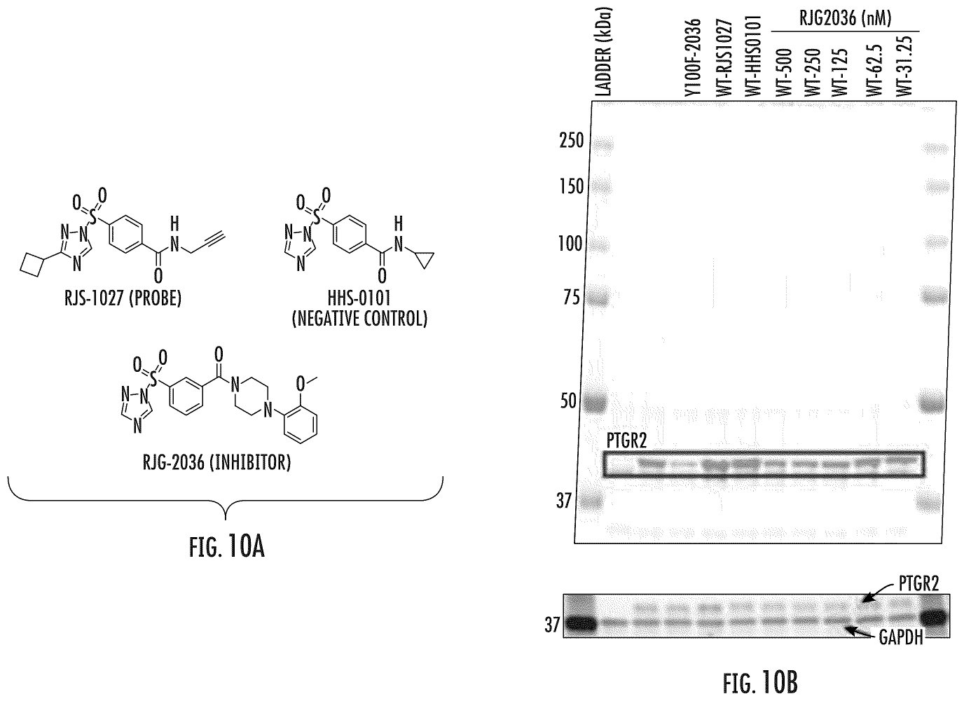

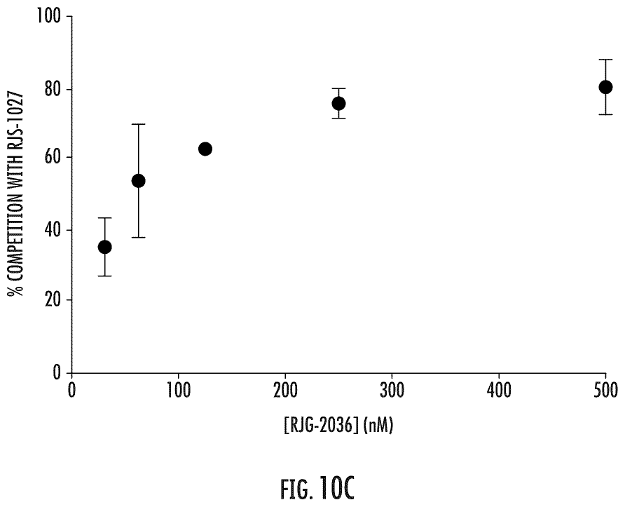

FIG. 10A shows the structures of compounds tested in situ: RJS-1027 as the probe for reporting inhibitor labeling of PTGR2, HHS-0101 as the negative control for trace PTGR2 labeling, and RJG-2036 as a potent inhibitor of probe labeling. FIG. 10B shows an image of an in situ gel-based chemical proteomics assay using RJS-1027 (1 μM) as a probe to measure competition of binding PTGR2 (see box) with RJG-2036 (dose response). Anti-flag and GAPDH western blots are shown below the long gel and were performed separately. Quantitation of RJG-2036 competition with probe labeling normalized to the PTGR2 WT with RJS-1027 as a probe is shown in FIG. 10C.

### Example 11

### PTGR2 Substrate Assay

HEK293T cells, transfected as described in the “Cell Treatments” section in Example 10 were harvested. As a negative control, the soluble fraction of proteome containing recombinant PTGR2 was heat-inactivated by heating to 95° C. for 5 minutes. An aliquot containing 0.22 mg of protein from the soluble fraction of mock proteome, proteome containing recombinant PTGR2, or proteome containing heat-inactivated recombinant PTGR2 were incubated with DMSO or compound at 37° C. for 30 minutes. The substrate assay was initiated by adding NADPH (1.1 mM), DTT (1.1 mM), EDTA (1.1 mM), and 15-Keto-PGE2 (0.022 mM). Samples were mixed and incubated at 37° C. for 30 minutes. The reaction was quenched by addition of a 2:1 mixture of chloroform and methanol with 50 ug/mL BHT. Samples were vortexed and chilled on ice for 20 minutes. The organic and aqueous layer were separated via centrifugation at 2,000×g for 4 minutes. The organic layer was collected, dried under a stream of N2 and resuspended in 100 μL of a 1:1 mixture of methanol and IPA. Samples were diluted to achieve a final concentration of 10 pmol/μL. Nano-electrospray ionization-LC-MS/MS analyses were performed using a Thermo-Finnigan LTQ Linear Ion Trap Mass Spectrometer (Thermo Finnigan LLC, San Jose, California, United States of America). LC conditions used the following gradient on a Thermofisher Easy nLC-1200 LC-MS system (Thermo Fisher Scientific, Waltham, Massachusetts, United States of America). Solvent A: 0.1% formic acid in water; Solvent B: 0.1% formic acid in 4:1 MeCN:H₂O): 0-2 min 50% B, 300 nlmin−1; 2-3 min 50-70% B; 3-23 min 70-80% B; 23-24 min 80-90% B; 24-29 min 90% B; 29-35 min 90-25% B. A top one acquisition MS method was used.

### Data Analysis

MS1 Chromatograms for 15-keto-PGE2 (RT: 8.79 min.; [M+H]⁺ (m/z)=351.5) and 13,14-dihydro-15keto-PGE2 (RT: 9.17 min.; [M+H]⁺ (m/z)=353.5) were integrated and area under peaks was quantified.

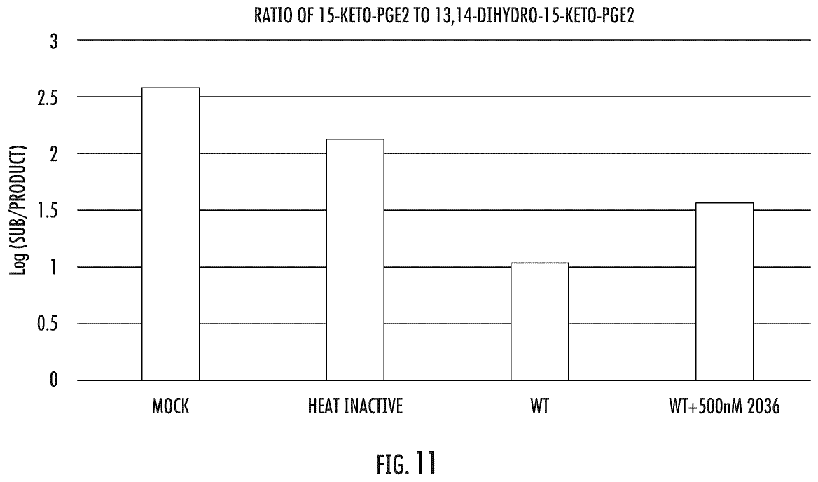

The enzymatic assay was performed to measure the ability of wild-type, recombinant PTGR2 (WT) to reduce its natural substrate (15-keto-PGE2) to 13,14-dihydro-15-keto-PGE2 in the absence (WT) or presence of inhibitor (500 nM RJG-2036) compared to heat-inactivated PTGR2 (Heat Inactive) and Mock, which did not overexpress recombinant PTGR2 and thus contains negligible PTGR2. Results are shown in FIG. 11.

### Example 12

### Additional Synthetic Methodology

### Preparation of Heteroarylbenzyl Sulfides (See Scheme 9, Above):

Heteroaryl bromide (1.0 equivalents), Pd₂(dba)₃ (0.05 equivalents), and XantPhos (0.10 equivalents) are added to a flask and purged with N2 via Schlenk technique. 1,4-dioxane (0.5 M relative to aryl bromide), benzyl mercaptan (1.0 equivalents), and DIEA (2.0 equivalents) were added. The reaction was heated to 110° C. for 2 hours. After cooling to room temperature, the reaction was concentrated in vacuo and purified by flash chromatography.

### Amidation of Heteroarylbenzyl Sulfide Intermediates Containing a Carboxylic Acid (See Also Scheme 10, Above):

To a solution of heteroarene (1.0 equivalents) in CH₂Cl₂ (1.0 M) was added HATU (1.0 equivalents), the corresponding amine (1.0 equivalents) and DIEA (1.0 equivalents. The reaction was stirred at room temperature for 16 hours, after which solvent was removed in vacuo and the product purified by flash chromatography.

### Preparation of Heteroarylbenzyl Sulfide Intermediates Containing an Aldehyde (Scheme 11, Above):

To a solution of heteroarylbenzyl sulfide (1.0 equivalents) in DCE (0.29 M) was added amine (1.0 equivalents), sodium triacetoxyborohydride (1.4 equivalents) and acetic acid (1.0 equivalents). The reaction was stirred at room temperature for 16 hours under a stream of N₂. The reaction was quenched with 1 M NaOH and the product extracted with diethyl ether. The organic layer was dried of sodium sulfate then concentrated in vacuo. The product was purified by flash chromatography.

### Synthesis of 1,2,4-Triazoles (See Scheme 7, Above):

Amide (1.0 equivalents) was dissolved in 1,4-dioxane (2.5 M). N,N-dimethylformamide dimethyl acetal (1.0 equivalents) was added and the reaction was heated to 110° C. for 2 hours. After cooling to room temperature, the reaction mixture was concentrated to dryness in vacuo, e.g., first on a rotary evaporator then on high-vacuum. The crude, dry residue was dissolved in acetic acid (1.3 M). Hydrazine hydrate (1.1 equivalents, 35% in water) was added and the reaction heated to 90° C. for 1 hour. After cooling to room temperature, the reaction was quenched with potassium hydroxide (1 M in methanol). After cooling to room temperature, solvent was removed in vacuo. The crude residue was dissolved in water and the aqueous layer extracted 5 times with dichloromethane. The combined organic layers were dried over sodium sulfate and concentrated in vacuo, affording the 1,2,4-triazole as a white solid.

Synthesis of Sulfonyl Triazoles from Sulfides (Scheme 9, Above):

To an ice-cold solution of heteroarylbenzyl sulfide (1.0 equivalents) in CH₃CN—HOAc-H₂O (40-1.5-1.0, 0.1 M relative to sulfide) was added portion-wise 2,4-dichloro-5,5-dimethylhydantoin (2.0 equivalents). The reaction mixture was stirred at 0-5° C. for 2 hours and concentrated in vacuo. The crude product was diluted with CH₂Cl₂ and the solution cooled down to ˜0° C. 5% NaHCO₃aqueous solution was added slowly at and the mixture was stirred at 0° C. for 15 minutes. The lower organic was washed once more with 10% brine solution at 0° C. The lower organic was dried over sodium sulfate. The crude solution was concentrated in vacuo and dissolved in EtOH (0.5 M relative to sulfide). 1,2,4-triazole (1.0 equivalents) and DIEA (1.0 equivalents) were added. Upon completion of the reaction, the mixture was concentrated in vacuo and the product purified by flash chromatography.

Synthesis of 1,3,5-triazine sulfonyl-triazole:

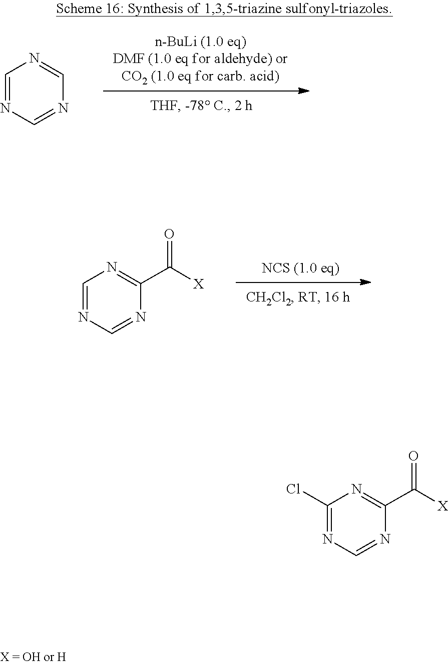

As shown in Scheme 16, 1,3,5-triazine (1.0 equivalents) was added to a flame-dried flask and purged with N₂ via Schlenk technique. THE (0.2 M) was added and the reaction cooled to −78° C. with a dry ice/acetone bath. After cooling for 15 minutes, n-BuLi (1.0 equivalents, 2.5 M in hexanes) was added dropwise. The reaction was stirred at −78° C. for 30 minutes. N,N-dimethylformamide (1.0 equivalents) or carbon dioxide (1 atm) was added to the reaction mixture. The reaction mixture was slowly warmed to room temperature overnight. The reaction mixture was concentrated in vacuo and the crude mixture carried forward without further purification. The crude residue was dissolved in CH₂Cl₂ (0.2 M). NCS (1.0 equivalents) was added and the reaction stirred at room temperature overnight. For the aldehyde: The reaction was concentrated in vacuo and the product purified by flash chromatography. For the carboxylic acid: The reaction was quenched with 1 M NaOH (1.0 equivalents). The aqueous layer was washed with CH₂Cl₂ three times. The aqueous layer was acidified with 1 M HCl and the precipitate filtered, affording the carboxylic acid which was carried forward to amidation (vide supra) without further purification.

### Synthesis of Amines:

### Buchwald-Hartwig Amination:

Boc-protected amine (1.0 equivalents), aryl bromide (1.0 equivalents), RuPhos (0.25 mol %), RuPhos Precatalyst (0.25 mol %), and sodium tert-butoxide (1.2 equivalents) were added to a flask and purged with N2 via Schlenk technique. 1,4-dioxane (0.5 M) was added and the reaction mixture was purged with N2 for 30 minutes at room temperature. The reaction mixture was submerged in a 100° C. oil bath and stirred overnight (16 hours). The reaction mixture was concentrated in vacuo and purified by flash chromatography.

### Chan-Evans-Lam Amine Synthesis:

Boc-protected amine (1.0 equivalents), boronic acid (2.0 equivalents), and copper acetate (1.0 equivalents) were added to a flame-dried flask containing activated molecular sieves (4 Å, 8-12 mesh, grade 514). Dry dichloromethane (0.6 M relative to amine) was added and the reaction was stirred at room temperature for 24 hours while exposed to air. The reaction mixture was diluted with dichloromethane and washed with water three times and once with brine. The organic layer was dried over sodium sulfate, then concentrated in vacuo. The product was purified by flash chromatography.

### Reductive Amination:

To a solution of aldehyde (1.0 equivalents) in DCE (0.29 M) was added boc-protected amine (1.0 equivalents), sodium triacetoxyborohydride (1.4 equivalents) and acetic acid (1.0 equivalents). The reaction was stirred at room temperature for 16 hours under a stream of N2. The reaction was quenched with 1 M NaOH and the product extracted with diethyl ether. The organic layer was dried of sodium sulfate then concentrated in vacuo. The product was purified by flash chromatography.

Synthesis of Difluoromethylene from Ketone:

Ketone (1.0 equivalents) was added to a plastic polypropylene container and dissolved in dichloromethane (0.5 M). DAST (2.0 equivalents) and pyridine hydrofluoride (1.3 equivalents) were added and the reaction stirred at room temperature overnight. The reaction was poured into cold, saturated sodium carbonate. The aqueous layer was extracted with dichloromethane three times. The combined organic layers were dried over sodium sulfate then concentrated in vacuo. The product was purified by flash chromatography.

### Deprotection of Boc-Protected Amines:

Boc-protected amine was dissolved in a mixture of dichloromethane and trifluoroacetic acid (4:1 CH₂Cl₂:TFA, 0.2 M relative to amine). The reaction was stirred overnight at room temperature. The reaction was quenched with 1 M KOH_{(aq)}. The aqueous layer was extracted five times with dichloromethane. The combine organic layers were dried over sodium sulfate, then concentrated in vacuo affording the amine product that was carried forward to amidation without further purification.

### Example 13

### Synthesis of Compounds 2040A, 2040B, and 2040C

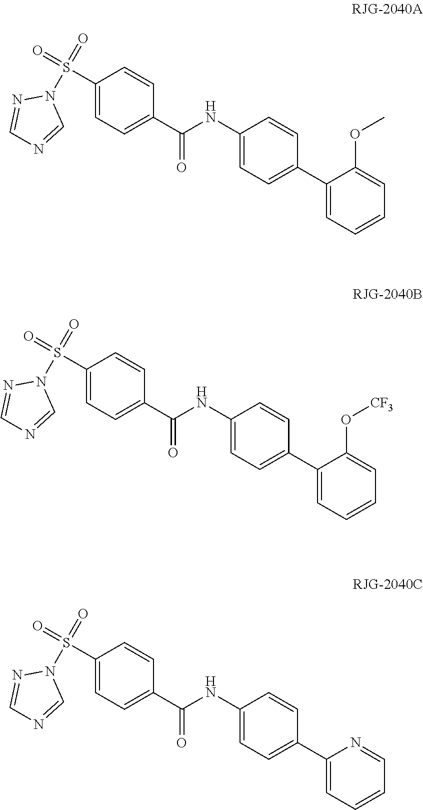 [SMILES: COc1ccccc1-c1ccc(NC(=O)c2ccc(S(=O)(=O)n3cncn3)cc2)cc1 ; O=C(Nc1ccc(-c2ccccc2OC(F)(F)F)cc1)c1ccc(S(=O)(=O)n2cncn2)cc1 ; O=C(Nc1ccc(-c2ccccn2)cc1)c1ccc(S(=O)(=O)n2cncn2)cc1]

The title compounds were prepared using the following steps:

### Suzuki-Miyaura Cross-Coupling:

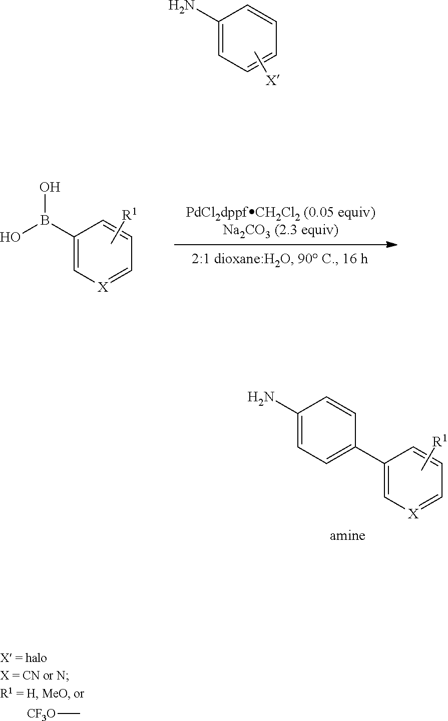

Aniline (1.0 equivalents), aryl boronic acid (1.0 equivalents), sodium carbonate (2.3 equivalents), and Pd(Cl)₂dppf CH₂Cl₂ (0.05 equivalents) were added to a flask and purged with N2 via Schlenk technique. 1,4-dioxane and water (2:1 mixture, 0.5 M) were added and the reaction mixture purged with N2 for 30 minutes. The reaction was submerged in a 90° C. oil bath and stirred overnight. The reaction mixture was diluted with dichloromethane and water. The aqueous layer was extracted three times with dichloromethane. The combined organic layers were dried over sodium sulfate then concentrated in vacuo. The product was purified by flash chromatography.

### Amidation:

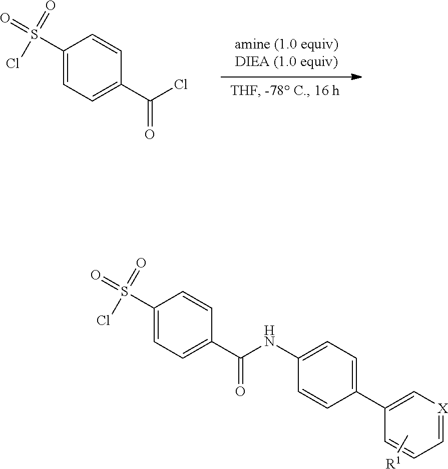

4-(chlorosulfonyl)benzoyl chloride (1.0 equivalents) was added to a flame-dried flask and purged with N2 via Schlenk technique. Dry THE (0.5 M) was added and the reaction mixture cooled to −78° C. with a dry ice/acetone bath. After cooling for 15 minutes, amine (1.0 equivalents) and DIEA (1.0 equivalents) were added. The reaction was slowly warmed to room temperature overnight. The reaction mixture was concentrated and the product purified by flash chromatography.

### Sulfonyl-Triazole Synthesis:

A flame-dried 6-dram vial fixed with a stir bar was charged with 1,2,4-triazole (1.0 equivalents) and purged with N2 via Schlenk technique. Dry THE (0.5 M) was added and the reaction stirred and cooled to 0° C. with an ice/water bath. After cooling for 10 minutes, sodium hydride (1.0 equivalents, 60% in parrafin oil) was added. The reaction was stirred at 0° C. for 30 minutes. The sulfonyl chloride (1.0 equivalents) from the amidation step was dissolved in dry THE (0.5 M). The sulfonyl chloride solution was added to the triazolide solution and the reaction slowly warmed to room temperature overnight. The reaction mixture was concentrated in vacuo and purified by flash chromatography.

### Characterization:

4-((1H-1,2,4-triazol-1-yl)sulfonyl)-N-(2′-methoxy-[1,1′-biphenyl]-4-yl)benzamide (RJG-2040A). White solid (194 mg, 90%). ¹H NMR (600 MHz, dmso) δ 10.60 (s, 1H), 9.49 (s, 1H), 8.40 (s, 1H), 8.26 (d, J=8.1 Hz, 2H), 8.20 (d, J=8.2 Hz, 2H), 7.77 (d, J=8.2 Hz, 2H), 7.48 (d, J=8.2 Hz, 2H), 7.38-7.26 (m, 2H), 7.11 (d, J=8.3 Hz, 1H), 7.03 (d, J=7.8 Hz, 1H), 3.77 (s, 3H). ¹³C NMR (201 MHz, dmso) δ 163.94, 156.14, 154.89, 146.76, 141.67, 137.41, 137.30, 130.20, 129.54, 129.43, 129.30, 128.71, 128.49, 120.79, 119.90, 111.77, 55.50. HPLC of RJG-2040A (see method of Example 9): Retention time (RT)=5.574 min.; 99.7% pure.

4-((1H-1,2,4-triazol-1-yl)sulfonyl)-N-(2′-(trifluoromethoxy)-[1,1′-biphenyl]-4-yl)-benzamide (RJG-2040B). White solid (168 mg, 78%). ¹H NMR (600 MHz, dmso) δ 10.67 (s, 1H), 9.49 (s, 1H), 8.41 (s, 1H), 8.29-8.24 (m, 2H), 8.23-8.18 (m, 2H), 7.88-7.84 (m, 2H), 7.58-7.53 (m, 1H), 7.50 (td, J=8.3, 7.3, 4.3 Hz, 5H). ¹⁹F NMR (564 MHz, dmso) δ−56.22. ¹³C NMR (201 MHz, dmso) δ 164.14, 154.90, 146.77, 145.28, 141.63, 138.37, 137.36, 134.30, 131.82, 131.60, 131.51, 129.45, 129.39, 129.21, 128.51, 128.04, 122.22, 121.73, 120.18, 120.00 (q, J=257.2 Hz). HPLC of RJG-2040B: RT=6.136 min.; 95.2% pure.

4-((1H-1,2,4-triazol-1-yl)sulfonyl)-N-(4-(pyridin-2-yl)phenyl)benzamide (RJG-2040C). White solid (63.8 mg, 56%). ¹H NMR (600 MHz, dmso) δ 10.68 (s, 1H), 9.49 (s, 1H), 8.91 (d, J=2.4 Hz, 1H), 8.55 (dd, J=4.8, 1.6 Hz, 1H), 8.41 (s, 1H), 8.27 (d, J=8.6 Hz, 2H), 8.21 (d, J=8.5 Hz, 2H), 8.08 (dt, J=7.8, 2.0 Hz, 1H), 7.94-7.85 (m, 2H), 7.79-7.75 (m, 2H), 7.48 (dd, J=8.0, 4.7 Hz, 1H). ¹³C NMR (201 MHz, dmso) δ 164.08, 154.89, 148.18, 147.34, 146.77, 141.55, 138.78, 137.37, 134.97, 133.71, 132.61, 129.46, 128.50, 127.19, 123.87, 120.75. HPLC of RJG-2040C: RT=3.660, 99.5% pure.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
[1] (a) M. J. Niphakis, B. F. Cravatt, Annual Review of Biochemistry 2014, 83, 341-377; (b) L. J. Keller, B. M. Babin, M. Lakemeyer, M. Bogyo, Curr Opin Chem Biol 2020, 54, 45-53.
[2] B. F. Cravatt, A. T. Wright, J. W. Kozarich, Annu Rev Biochem 2008, 77, 383-414.
[3] (a) Y. Liu, M. P. Patricelli, B. F. Cravatt, Proc Natl Acad Sci USA 1999, 96, 14694-14699; (b) M. P. Patricelli, D. K. Giang, L. M. Stamp, J. J. Burbaum, Proteomics 2001, 1, 1067-1071; (c) D. Kidd, Y. Liu, B. F. Cravatt, Biochemistry 2001, 40, 4005-4015.
[4] J. S. Cisar, O. D. Weber, J. R. Clapper, J. L. Blankman, C. L. Henry, G. M. Simon, J. P. Alexander, T. K. Jones, R. A. B. Ezekowitz, G. P. O'Neill, C. A. Grice, J Med Chem 2018, 61, 9062-9084.
[5] (a) G. M. Simon, B. F. Cravatt, J Biol Chem 2010, 285, 11051-11055; (b) J. Z. Long, B. F. Cravatt, Chem Rev 2011, 111, 6022-6063; (c) D. A. Bachovchin, B. F. Cravatt, Nat Rev Drug Discov 2012, 11, 52-68.
[6] (a) S. M. Hacker, K. M. Backus, M. R. Lazear, S. Forli, B. E. Correia, B. F. Cravatt, Nature chemistry 2017, 9, 1181-1190; (b) Q. Zhao, X. Ouyang, X. Wan, K. S. Gajiwala, J. C. Kath, L. H. Jones, A. L. Burlingame, J. Taunton, J Am Chem Soc 2017, 139, 680-685; (c) M. P. Patricelli, T. K. Nomanbhoy, J. Wu, H. Brown, D. Zhou, J. Zhang, S. Jagannathan, A. Aban, E. Okerberg, C. Herring, B. Nordin, H. Weissig, Q. Yang, J. D. Lee, N. S. Gray, J. W. Kozarich, Chem Biol 2011, 18, 699-710; (d) D. A. Shannon, R. Banerjee, E. R. Webster, D. W. Bak, C. Wang, E. Weerapana, J Am Chem Soc 2014, 136, 3330-3333.
[7] (a) E. Weerapana, C. Wang, G. M. Simon, F. Richter, S. Khare, M. B. Dillon, D. A. Bachovchin, K. Mowen, D. Baker, B. F. Cravatt, Nature 2010, 468, 790-795; (b) K. M. Backus, B. E. Correia, K. M. Lum, S. Forli, B. D. Horning, G. E. Gonzalez-Paez, S. Chatterjee, B. R. Lanning, J. R. Teijaro, A. J. Olson, D. W. Wolan, B. F. Cravatt, Nature 2016, 534, 570-574; (c) J. M. Bradshaw, J. M. McFarland, V. O. Paavilainen, A. Bisconte, D. Tam, V. T. Phan, S. Romanov, D. Finkle, J. Shu, V. Patel, T. Ton, X. Li, D. G. Loughhead, P. A. Nunn, D. E. Karr, M. E. Gerritsen, J. O. Funk, T. D. Owens, E. Verner, K. A. Brameld, R. J. Hill, D. M. Goldstein, J. Taunton, Nat Chem Biol 2015, 11, 525-531; (d) E. Yoo, B. H. Stokes, H. de Jong, M. Vanaerschot, T. Kumar, N. Lawrence, M. Njoroge, A. Garcia, R. Van der Westhuyzen, J. D. Momper, C. L. Ng, D. A. Fidock, M. Bogyo, J Am Chem Soc 2018, 140, 11424-11437; (e) E. V. Vinogradova, X. Zhang, D. Remillard, D. C. Lazar, R. M. Suciu, Y. Wang, G. Bianco, Y. Yamashita, V. M. Crowley, M. A. Schafroth, M. Yokoyama, D. B. Konrad, K. M. Lum, G. M. Simon, E. K. Kemper, M. R. Lazear, S. Yin, M. M. Blewett, M. M. Dix, N. Nguyen, M. N. Shokhirev, E. N. Chin, L. L. Lairson, B. Melillo, S. L. Schreiber, S. Forli, J. R. Teijaro, B. F. Cravatt, Cell 2020, 182, 1009-1026 e1029.
[8] (a) H. S. Hahm, E. K. Toroitich, A. L. Borne, J. W. Brulet, A. H. Libby, K. Yuan, T. B. Ware, R. L. McCloud, A. M. Ciancone, K. L. Hsu, Nat Chem Biol 2020, 16, 150-159; (b) W. Chen, J. Dong, L. Plate, D. E. Mortenson, G. J. Brighty, S. Li, Y. Liu, A. Galmozzi, P. S. Lee, J. J. Hulce, B. F. Cravatt, E. Saez, E. T. Powers, I. A. Wilson, K. B. Sharpless, J. W. Kelly, J Am Chem Soc 2016, 138, 7353-7364; (c) L. A. Crawford, E. Weerapana, Mol Biosyst 2016, 12, 1768-1771; (d) C. Gu, D. A. Shannon, T. Colby, Z. Wang, M. Shabab, S. Kumari, J. G. Villamor, C. J. McLaughlin, E. Weerapana, M. Kaiser, B. F. Cravatt, R. A. van der Hoorn, Chem Biol 2013, 20, 541-548; (e) E. C. Hett, H. Xu, K. F. Geoghegan, A. Gopalsamy, R. E. Kyne, Jr., C. A. Menard, A. Narayanan, M. D. Parikh, S. Liu, L. Roberts, R. P. Robinson, M. A. Tones, L. H. Jones, ACS Chem Biol 2015, 10, 1094-1098; (f) A. Narayanan, L. H. Jones, Chem Sci 2015, 6, 2650-2659; (g) H. Ban, J. Gavrilyuk, C. F. Barbas, 3rd, J Am Chem Soc 2010, 132, 1523-1525.
[9] (a) A. A. Antolin, J. E. Tym, A. Komianou, I. Collins, P. Workman, B. Al-Lazikani, Cell Chem Biol 2018, 25, 194-205 e195; (b) A. L. Hopkins, C. R. Groom, Nat Rev Drug Discov 2002, 1, 727-730; (c) B. Munos, Nat Rev Drug Discov 2009, 8, 959-968; (d) A. J. Carter, O. Kraemer, M. Zwick, A. Mueller-Fahrnow, C. H. Arrowsmith, A. M. Edwards, Drug Discov Today 2019, 24, 2111-2115.
[10] (a) J. W. Brulet, A. L. Borne, K. Yuan, A. H. Libby, K. L. Hsu, J Am Chem Soc 2020, 142, 8270-8280; (b) T. Huang, S. Hosseinibarkooie, A. L. Borne, M. E. Granade, J. W. Brulet, T. E. Harris, H. A. Ferris, K.-L. Hsu, Chemical Science 2021, 12, 3295-3307.
[11] (a) D. A. Erlanson, S. W. Fesik, R. E. Hubbard, W. Jahnke, H. Jhoti, Nat Rev Drug Discov 2016, 15, 605-619; (b) A. L. Hopkins, G. M. Keseru, P. D. Leeson, D. C. Rees, C. H. Reynolds, Nat Rev Drug Discov 2014, 13, 105-121; (c) D. E. Scott, A. G. Coyne, S. A. Hudson, C. Abell, Biochemistry 2012, 51, 4990-5003.
[12] C. G. Parker, A. Galmozzi, Y. Wang, B. E. Correia, K. Sasaki, C. M. Joslyn, A. S. Kim, C. L. Cavallaro, R. M. Lawrence, S. R. Johnson, I. Narvaiza, E. Saez, B. F. Cravatt, Cell 2017, 168, 527-541 e529.
[13] M. Mann, Nat Rev Mol Cell Biol 2006, 7,952-958.
[14] V. V. Rostovtsev, L. G. Green, V. V. Fokin, K. B. Sharpless, Angewandte Chemie International Edition 2002, 41, 2596-2599.
[15] M. Bern, Y. J. Kil, C. Becker, Curr Protoc Bioinformatics 2012, Chapter 13, Unit 13-20.
[16] A. Bergeron, M. D'Astous, D. E. Timm, R. M. Tanguay, J Biol Chem 2001, 276, 15225-15231.
[17] Y. H. Wu, T. P. Ko, R. T. Guo, S. M. Hu, L. M. Chuang, A. H. Wang, Structure 2008, 16, 1714-1723.
[18] H. S. Hansen, in Methods in Enzymology, Vol. 86, Academic Press, 1982, pp. 156-163.
[19] A. L. Borne, J. W. Brulet, K. Yuan, K.-L. Hsu, RSC Chemical Biology 2021.
[20] W. L. Chou, L. M. Chuang, C. C. Chou, A. H. Wang, J. A. Lawson, G. A. FitzGerald, Z. F. Chang, J Biol Chem 2007, 282, 18162-18172.
[21] E. Y. Chang, Y. C. Chang, C. T. Shun, Y. W. Tien, S. H. Tsai, S. W. Hee, I. J. Chen, L. M. Chuang, PloS one 2016, 11, e0147390.
[22] C. E. Franks, S. T. Campbell, B. W. Purow, T. E. Harris, K. L. Hsu, Cell Chem Biol 2017, 24, 870-880 e875.
[23] D. S. Cao, N. Xiao, Q. S. Xu, A. F. Chen, Bioinformatics 2015, 31, 279-281.
[24] J. Dong, M. F. Zhu, Y. H. Yun, A. P. Lu, T. J. Hou, D. S. Cao, Briefings in bioinformatics 2021, 22, 474-484.
[25] N. M. O'Boyle, M. Banck, C. A. James, C. Morley, T. Vandermeersch, G. R. Hutchison, J Cheminform 2011, 3, 33.
[26] Lin Y-I, Lang S A, Lovell M F, Perkinson N A. New synthesis of 1,2,4-triazoles and 1,2,4-oxadiazoles. The Journal of Organic Chemistry 1979, 44(23): 4160-4164.
[27] Lazreg, F., et. al., Organometallics, 37, 679-683, 2018.
[28] Yamauchi, M., et. al., Heterocycles, 80(1), 177-181, 2010.

It will be understood that various details of the presently disclosed subject matter can be changed without departing from the scope of the presently disclosed subject matter. Furthermore, the foregoing description is for the purpose of illustration only, and not for the purpose of limitation.

1. A compound having a structure of Formula (I): 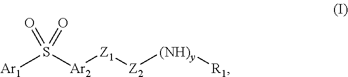
wherein:
y is 0 or 1;
Ar₁ is selected from the group consisting of triazole, substituted triazole, imidazole, substituted imidazole, pyrazole, substituted pyrazole, tetrazole, and substituted tetrazole;
Ar₂ is aryl or heteroaryl;
Z₁ is —CH₂— or —C(═O)—;
Z₂ is a heterocyclic, heteroaryl, substituted heterocyclic or substituted heteroaryl group; and
R₁ is aryl, substituted aryl, heteroaryl or substituted heteroaryl;
subject to the proviso that when Z₂ is piperidinyl, y is 1 and that when Z₁ is —C(═O)— and
Z₂ is piperazinyl, R₁ is not substituted pyrimidinyl;
or a pharmaceutically acceptable salt thereof.

2. The compound of claim 1, wherein Ar₂ is selected from phenyl, pyridinyl, pyrimidinyl, and triazinyl.

3. The compound of claim 1, wherein Ar₁ is 1,2,4-triazole or a substituted 1,2,4-triazole group.

4. The compound of claim 1, wherein Z₂ is selected from the group consisting of piperazinyl, piperidinyl, tetrahydrofuranyl, pyrrolidinyl, pyrrolyl, furanyl, diazepanyl, azetidinyl, and 2,6-diaza[3.3]heptanyl.

5. The compound of claim 1, wherein R₁ is an optionally substituted aryl or heteroaryl group, wherein said aryl or heteroaryl group is selected from phenyl, pyridyl, indolinyl, tetrahydroquinolinyl, indolyl, benzofuranyl, indanyl, dihydrobenzofuranyl, chromanyl, benzofuranyl, anthranilyl, benzofurazanyl, isoindolyl, oxindolyl, and isocarbostyrilyl.

6. The compound of claim 1, wherein the compound having a structure of Formula (I) has a structure of Formula (II): 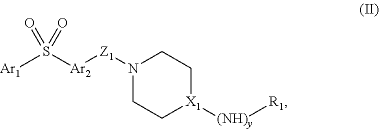
wherein:
y is 0 or 1;
Ar₁ is selected from the group consisting of triazole, substituted triazole, imidazole, substituted imidazole, pyrazole, substituted pyrazole, tetrazole, and substituted tetrazole;
Ar₂ is aryl or heteroaryl;
Z₁ is —CH₂— or —C(═O)—;
X₁ is N or CH; and
R₁ is aryl, substituted aryl, heteroaryl or substituted heteroaryl;
subject to the proviso that when X₁ is CH, y is 1 and when X₁ is N, y is 0; or
a pharmaceutically acceptable salt thereof.

7. The compound of claim 1, wherein Z₁ is —C(═O)—.

8. The compound of claim 1, wherein the compound having a structure of Formula (I) has a structure of Formula (III): 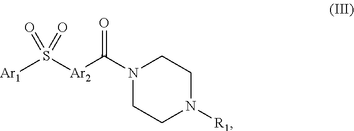
wherein:
A₁ is selected from the group consisting of triazole, substituted triazole, imidazole, substituted imidazole, pyrazole, substituted pyrazole, tetrazole, and substituted tetrazole;
Ar₂ is aryl or heteroaryl; and
R₁ is aryl, heteroaryl, substituted aryl, or substituted heteroaryl, optionally phenyl, substituted phenyl, pyridyl, or substituted pyridyl;
or a pharmaceutically acceptable salt thereof.

9. The compound of claim 8, wherein Ar₂ is phenyl and the compound of Formula (III) has a structure of Formula (IIIa) or Formula (IIIb): 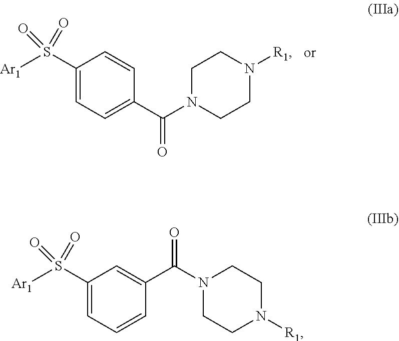
wherein:
Ar₁ is selected from the group consisting of triazole, substituted triazole, imidazole, substituted imidazole, pyrazole, substituted pyrazole, tetrazole, and substituted tetrazole; and
R₁ is aryl, heteroaryl, substituted aryl, or substituted heteroaryl, optionally phenyl, substituted phenyl, pyridyl, or substituted pyridyl;
or a pharmaceutically acceptable salt thereof.

10. The compound of claim 8, wherein Ar₁ is a 1,2,4-triazole or substituted 1,2,4-triazole group and the compound of Formula (III) has a structure of Formula (IV): 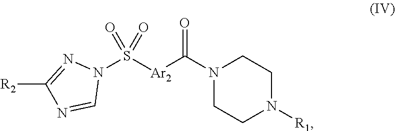
wherein:
Ar₂ is aryl or heteroaryl;
R₁ is aryl, substituted aryl, heteroaryl, or substituted heteroaryl, optionally phenyl, substituted phenyl, pyridyl, or substituted pyridyl; and
R₂ is selected from H, alkyl, cycloalkyl, aryl, and substituted aryl;
or a pharmaceutically acceptable salt thereof.

11. The compound of claim 10, wherein the compound of Formula (IV) has a structure of Formula (IVa) or (IVb): 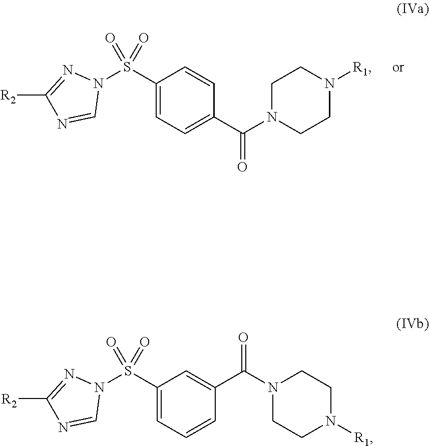
wherein:
R₁ is phenyl, substituted phenyl, pyridyl, or substituted pyridyl; and
R₂ is selected from H, alkyl, cycloalkyl, aryl, and substituted aryl;
or a pharmaceutically acceptable salt thereof.

12. The compound of claim 10, wherein R₂ is selected from the group consisting of H, cycloalkyl, phenyl, furanyl, pyridyl, and substituted phenyl.

13. The compound of claim 10, wherein R₂ is substituted phenyl, wherein said substituted phenyl is phenyl substituted with one or more substituent selected from the group consisting of halo, perfluoroalkyl, alkoxy, perfluoralkoxy, and aryl.

14. The compound of claim 10, wherein R₂ is selected from H, 2-pyridyl, and 2-methoxyphenyl.

15. The compound of claim 1, wherein R₁ is substituted phenyl or substituted pyridyl, wherein said substituted phenyl or substituted pyridyl are phenyl or pyridyl substituted with one or more substituent selected from the group consisting of alkyl, halo, haloalkyl, alkoxy, acyl, —C(═O)—NH₂, amino, alkylamino, and dialkylamino, optionally alkyl, halo, and alkoxy.

16. The compound of claim 1, wherein R₁ is substituted phenyl, optionally wherein R₁ is phenyl substituted by one or more substituent selected from halo and alkoxy.

17. The compound of claim 1, wherein R₁ is alkoxy-substituted phenyl, optionally methoxy-substituted phenyl.

18. The compound of claim 1, wherein R₁ is 2-methoxyphenyl.

19. The compound of claim 1, wherein the compound is selected from the group consisting of:
(4-((1H-1,2,4-triazol-1-yl)sulfonyl)phenyl)(4-phenylpiperazin-1-yl)methanone (AMC-0702);
(4-((1H-1,2,4-triazol-1-yl)sulfonyl)phenyl)(4-(2-methoxyphenyl)piperazin-1-yl)methanone (AMC-0703);
(4-(2-methoxyphenyl)piperazin-1-yl)(4-((3-phenyl-1H-1,2,4-triazol-1-yl)sulfonyl)-phenyl)methanone (RJG-1101);
(4-((1H-1,2,4-triazol-1-yl)sulfonyl)phenyl)(4-(3-methoxyphenyl)piperazin-1-yl)-methanone (RJG-1103);
(4-((1H-1,2,4-triazol-1-yl)sulfonyl)phenyl)(4-(4-methoxyphenyl)piperazin-1-yl)-methanone (RJG-1105);
(4-((3-(4-bromophenyl)-1H-1,2,4-triazol-1-yl)sulfonyl)phenyl)(4-(2-methoxy-phenyl)piperazin-1-yl)methanone (RJG-1112);
(4-((3-(4-fluorophenyl)-1H-1,2,4-triazol-1-yl)sulfonyl)phenyl)(4-(2-methoxy-phenyl)piperazin-1-yl)methanone (RJG-1114);
(4-(2-methoxyphenyl)piperazin-1-yl)(4-((3-(4-(trifluoromethyl)phenyl)-1H-1,2,4-triazol-1-yl)sulfonyl)phenyl)methanone (RJG-1115);
(4-((3-(furan-2-yl)-1H-1,2,4-triazol-1-yl)sulfonyl)phenyl)(4-(2-methoxyphenyl)-piperazin-1-yl)methanone RJG-1185, (4-((3-(2-fluorophenyl)-1H-1,2,4-triazol-1-yl)sulfonyl)phenyl)(4-(2-methoxy-phenyl)piperazin-1-yl)methanone (RJG-1187);
(4-((3-(3-fluorophenyl)-1H-1,2,4-triazol-1-yl)sulfonyl)phenyl)(4-(2-methoxy-phenyl)piperazin-1-yl)methanone (RJG-1188);
(4-(2-methoxyphenyl)piperazin-1-yl)(4-((3-(pyridin-3-yl)-1H-1,2,4-triazol-1-yl)-sulfonyl)phenyl)methanone (RJG-1189);
(4-(2-methoxyphenyl)piperazin-1-yl)(4-((3-(4-(trifluoromethoxy)phenyl)-1H-1,2,4-triazol-1-yl)sulfonyl)phenyl)methanone (RJG-1228);
(4-(4-iodophenyl)piperazin-1-yl)(4-((3-phenyl-1H-1,2,4-triazol-1-yl)sulfonyl)-phenyl)methanone (RJG-1257);
(4-((1H-1,2,4-triazol-1-yl)sulfonyl)phenyl)(4-(pyridin-2-yl)piperazin-1-yl)-methanone (RJG-1285);
(4-(2-methoxyphenyl)piperazin-1-yl)(4-((3-(pyridin-4-yl)-1H-1,2,4-triazol-1-yl)-sulfonyl)phenyl)methanone (RJG-1291);
(4-((3-([1,1′-biphenyl]-4-yl)-1H-1,2,4-triazol-1-yl)sulfonyl)phenyl)(4-(2-methoxy-phenyl)piperazin-1-yl)methanone (RJG-2011);
(3-((1H-1,2,4-triazol-1-yl)sulfonyl)phenyl)(4-(2-methoxyphenyl)piperazin-1-yl)-methanone (RJG-2036);
(4-((3-cyclopropyl-1H-1,2,4-triazol-1-yl)sulfonyl)phenyl)(4-(2-methoxyphenyl)-piperazin-1-yl)methanone (RJG-2048);
4-((3-cyclobutyl-1H-1,2,4-triazol-1-yl)sulfonyl)phenyl)(4-(2-methoxyphenyl)-piperazin-1-yl)methanone (RJG-2049);
(4-((3-cyclopentyl-1H-1,2,4-triazol-1-yl)sulfonyl)phenyl)(4-(2-methoxyphenyl)-piperazin-1-yl)methanone (RJG-2050);
(4-((3-cyclohexyl-1H-1,2,4-triazol-1-yl)sulfonyl)phenyl)(4-(2-methoxyphenyl)-piperazin-1-yl)methanone (RJG-2051);
(4-((3-(4-methoxyphenyl)-1H-1,2,4-triazol-1-yl)sulfonyl)phenyl)(4-(2-methoxy-phenyl)piperazin-1-yl)methanone (RJG-2056); and
(4-((1H-1,2,4-triazol-1-yl)sulfonyl)phenyl)(4-((2-methoxyphenyl)amino)piperidin-1-yl)methanone (RJG-2058);
or a pharmaceutically acceptable salt thereof.

20. The compound of claim 19, wherein the compound is selected from (4-((1H-1,2,4-triazol-1-yl)sulfonyl)phenyl)(4-(2-methoxyphenyl)piperazin-1-yl)methanone (AMC-0703), (4-(2-methoxyphenyl)piperazin-1-yl)(4-((3-(pyridin-3-yl)-1H-1,2,4-triazol-1-yl)-sulfonyl)-phenyl)methanone (RJG-1189), and (3-((1H-1,2,4-triazol-1-yl)sulfonyl)phenyl)-(4-(2-methoxyphenyl)piperazin-1-yl)-methanone (RJG-2036); or a pharmaceutically acceptable salt thereof.

21. A pharmaceutical composition comprising a compound of claim 1 and a pharmaceutically acceptable carrier.

22. A method of inhibiting prostaglandin reductase 2 (PTGR2), wherein the method comprises contacting a sample comprising PTGR2 with an effective amount of a compound of claim 1.

23. The method of claim 22, wherein the sample comprising PTGR2 is a biological sample selected from a biological fluid, a cell culture, a cell extract, a tissue, a tissue extract, an organ, or an organism.

24. A probe compound, wherein the probe compound has a structure of Formula (V): 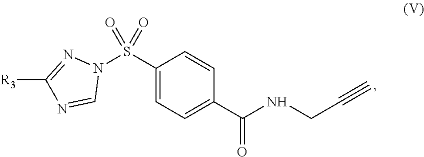
wherein:
R₃ is selected from cyclopropyl, cyclobutyl, cyclopentyl, and cyclohexyl.

25. A method of identifying a reactive tyrosine and/or a reactive lysine of a protein, the method comprising:
(a) providing a protein sample comprising isolated proteins, living cells, or a cell lysate;
(b) contacting the protein sample with a probe compound of claim 24 for a period of time sufficient for the probe compound to react with at least one reactive tyrosine and/or at least one reactive lysine in a protein in the protein sample, thereby forming at least one modified reactive tyrosine residue and/or at least one modified reactive lysine residue; and
(c) analyzing proteins in the protein sample to identify at least one modified tyrosine residue and/or at least one modified lysine residue, thereby identifying at least one reactive tyrosine and/or at least one reactive lysine of a protein; wherein the at least one modified reactive tyrosine residue comprises a modified tyrosine residue comprising a structure of Formula (VI): 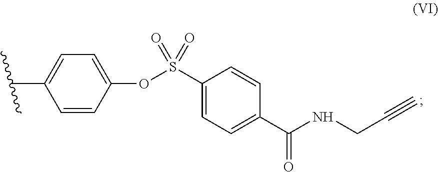 [SMILES: *c1ccc(OS(=O)(=O)c2ccc(C(=O)NCC#C)cc2)cc1]
and the at least one modified reactive lysine residue comprises a modified lysine residue comprising a structure of Formula (VII):

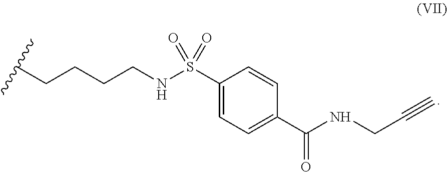 [SMILES: *CCCCNS(=O)(=O)c1ccc(C(=O)NCC#C)cc1]

26. The method of claim 25, wherein the analyzing of step (c) further comprises tagging the at least one modified reactive tyrosine residue and/or the at least one modified reactive lysine residue with a compound comprising a detectable labeling group, thereby forming at least one tagged reactive tyrosine residue comprising said detectable labeling group and/or at least one tagged reactive lysine residue comprising said detectable labeling group, optionally wherein the detectable labeling group comprises biotin or a biotin derivative, optionally wherein the biotin derivative is desthiobiotin.

27. The method of claim 25, wherein the tagging comprises reacting an alkyne group of the at least one tagged reactive tyrosine residue and/or at least one tagged reactive lysine residue with a compound comprising an (i) an azide moiety and (ii) the detectable labeling group, optionally via a copper-catalyzed azide-alkyne cycloaddition (CuAAC) coupling reaction.

28. The method of claim 25, wherein the analyzing further comprises digesting the protein sample with trypsin to provide a digested protein sample comprising a protein fragment comprising the at least one tagged reactive tyrosine moiety comprising the detectable group and/or a protein fragment comprising the at least one tagged reactive lysine moiety comprising the detectable group.

29. The method of claim 28, wherein the analyzing further comprises enriching the digested protein sample for the detectable labeling group, optionally wherein the enriching comprises contacting the digested protein sample with a solid support comprising a binding partner of the detectable labeling group.

30. The method of claim 29, wherein the analyzing further comprises analyzing the enriched the digested protein sample via liquid chromatography-mass spectrometry.

31. A compound having a structure of Formula (I′): 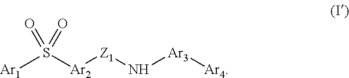
wherein:
Ar₁ is selected from the group consisting of triazole, substituted triazole, imidazole, substituted imidazole, pyrazole, substituted pyrazole, tetrazole, and substituted tetrazole;
Ar₂ is aryl or heteroaryl;
Z₁ is —CH₂— or —C(═O)—;
Ar₃ is aryl or heteroaryl; and
Ar₄ is substituted aryl, heteroaryl, or substituted heteroaryl;
or a pharmaceutically acceptable salt thereof.

32. The compound of claim 31, wherein Ar₁ is 1,2,4-triazole or substituted 1,2,4-triazole.

33. The compound of claim 31, wherein Z₁ is —C(═O)—.

34. The compound of claim 31, wherein Ar₃ is phenyl.

35. The compound of claim 31, wherein Ar₄ is substituted phenyl or pyridyl.

36. The compound of claim 31, wherein the compound is selected from the group consisting of:
4-((1H-1,2,4-triazol-1-yl)sulfonyl)-N-(2′-methoxy-[1,1′-biphenyl]-4-yl)benzamide (RJG-2040A),
4-((1H-1,2,4-triazol-1-yl)sulfonyl)-N-(2′-(trifluoromethoxy)-[1,1′-biphenyl]-4-yl)-benzamide (RJG-2040B), and
4-((1H-1,2,4-triazol-1-yl)sulfonyl)-N-(4-(pyridin-2-yl)phenyl)benzamide (RJG-2040C);
or a pharmaceutically acceptable salt thereof.

37. A pharmaceutical composition comprising a compound of claim 31 and a pharmaceutically acceptable carrier.

38. A method of inhibiting prostaglandin reductase 2 (PTGR2), wherein the method comprises contacting a sample comprising PTGR2 with an effective amount of a pharmaceutical composition of claim 21.
